(19) United States
(12) Patent Application Publication
(10) Pub. No.: US 2022/0362262 A1
(43) Pub. Date: Nov. 17, 2022

(54) METHODS AND COMPOSITIONS FOR TISSUE REGENERATION

(71) Applicant: Institute of Zoology, Chinese Academy of Sciences, Beijing (CN)

(72) Inventors: Shyh Chang NG, Beijing (CN); Tao Yan LIU, Beijing (CN); Lan Fang LUO, Beijing (CN); Kun LIANG, Beijing (CN); Wen Wu MA, Beijing (CN)

(21) Appl. No.: 17/761,187

(22) PCT Filed: Sep. 16, 2020
(86) PCT No.: PCT/CN2020/115635

§ 371 Date: Mar. 16, 2022

(30) Foreign Application Priority Data
Sep. 16, 2019 (CN) PCT/CN2019/105890

(51) Int. Cl. A61K 31/5575; A61K 31/4439; A61K 31/426; A61P 21/06

(52) U.S. Cl. CPC A61K 31/5575; A61K 31/4439; A61K 31/426; A61P 21/06

(57) ABSTRACT

Provided are methods and compositions for promoting tissue (e.g., muscle) regeneration using one or more activators of fatty acid oxidation, such as one or more PPARγ activators. The methods and compositions described herein are also useful for promoting tissue growth, inducing proliferation of stem cells, inducing differentiation of tissuegenic cells (e.g., myogenic cells), and treating a disease or condition associated with a tissue (e.g., muscle), such as tissue injury, degeneration or aging, in an individual.

### CROSS-REFERENCE TO RELATED APPLICATIONS

This application claims priority benefit of International Patent Application No. PCT/CN2019/105890 filed Sep. 16, 2019, the contents of which are incorporated herein by reference in their entirety.

### FIELD

The present application relates to use of fatty acid oxidation activators to enhance tissue (e.g., muscle) regeneration.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 182452000341SEQLIST.TXT, date recorded: Sep. 14, 2020, size: 5 KB).

### BACKGROUND

Substrate and oxygen availability, as well as bioenergetic demand, are known to determine which metabolic pathway is used to generate ATP. Under hypoxic conditions, ATP is generated mainly via glycolysis, which uses glucose or fructose. However, under aerobic conditions ATP is produced mainly via oxidative phosphorylation (OxPhos). Mitochondrial OxPhos requires the Krebs cycle, which uses carboxylic acids derived from sugars, amino acids or fatty acids to produce NADH and FADH₂, and the electron transport chain (ETC), which oxidizes NADH and FADH₂ to generate a proton gradient to drive ATP synthesis. Compared to OxPhos, glycolysis is a less efficient method to generate ATP per carbon. However glycolysis provides a number of important advantages for proliferative cells, including cancer cells and stem/progenitor cells, e.g. the ability to rapidly generate the necessary glycolytic intermediates for the biosynthesis of new macromolecules essential for cell proliferation (Lunt and Vander Heiden, 2011; Shyh-Chang et al., 2013; Ryall and Sartorelli, 2015; Shyh-Chang and Ng, 2017).

While much effort has been expended on discovering the metabolic pathways that fuel cell proliferation, less is known about the metabolic pathways that inhibit proliferation and promote cellular differentiation. Both cellular proliferation and differentiation are necessary for tissue regeneration. Skeletal muscles are a well-established model system for these processes (Comai and Tajbakhsh, 2014; Lepper et al., 2011; Murphy et al., 2011). In response to injury, quiescent muscle stem cells activate to enter a highly proliferative state (Gunther et al., 2013; Lepper et al., 2009; Relaix et al., 2006; Sambasivan et al., 2011; Seale et al., 2000; von Maltzahn et al., 2013; Gayraud-Morel et al., 2012). Such activated muscle stem cells or progenitors are called myoblasts, marked and regulated by the muscle-specific transcription factor MyoD (MYOD1). Upon commitment to the differentiation program, myoblasts then express myogenin (MYOG) and differentiate into non-proliferative myocytes. These MYOG+ myocytes are fusion-competent and subsequently fuse into multi-nucleated myotubes, which express high levels of myosin heavy chain (MHC) and sarcomeric α-actinin to form the highly specialized striated muscle cytokeleton, to repair damaged muscles and regenerate new muscle fibers. Due to the complexity of molecular changes that occur in myoblasts during the transition from cellular proliferation to differentiation, the metabolic requirements of these cellular states are likely to change dramatically as well. However, most previous studies of myoblast differentiation had tended to focus on multi-nucleated myotubes as the endpoint, and often neglected the intermediate endpoint of non-proliferative, stably committed, mononucleated myocytes. Many studies had also utilized the immortalized C2C12 cell-line, instead of primary muscle cells, with differing results. Thus, the truly causal events that trigger cell fate transition in the earliest phases of primary myoblast differentiation might still be unclear.

This disclosure of all publications, patents, patent applications and published patent applications referred to herein are hereby incorporated herein by reference in their entirety.

### BRIEF SUMMARY

The present application provides compositions and methods of using fatty acid oxidation (“FAO”) activators for tissue (e.g., muscle) regeneration and therapy.

One aspect of the present application provides a method of promoting regeneration of a tissue (e.g., muscle tissue), comprising contacting the tissue with one or more FAO activators. In some embodiments, the tissue is contacted with the one or more FAO activators for no more than about 72 hours, no more than about 48 hours, or no more than about 24 hours. In some embodiments, the contacting is in vitro, ex vivo or in vivo. In some embodiments, the tissue is selected from the group consisting of a muscle tissue, a liver tissue, a heart tissue, a skin tissue and a hair follicle. In some embodiments, the tissue is a muscle tissue.

One aspect of the present application provides a method of promoting growth of a tissue (e.g., muscle tissue), comprising contacting the tissue with one or more FAO activators. In some embodiments, the tissue is contacted with the one or more FAO activators for no more than about 72 hours, no more than about 48 hours, or no more than about 24 hours. In some embodiments, the contacting is in vitro, ex vivo or in vivo. In some embodiments, the tissue is selected from the group consisting of a muscle tissue, a liver tissue, a heart tissue, a skin tissue and a hair follicle. In some embodiments, the tissue is a muscle tissue.

One aspect of the present application provides a method of inducing differentiation of tissuegenic cells (e.g., myogenic cells) in a tissue (e.g., muscle tissue), comprising contacting the tissue with one or more FAO activators. One aspect of the present application provides a method of inducing maturation of tissuegenic cells (e.g., myogenic cells) in a tissue (e.g., muscle tissue), comprising contacting the tissue with one or more FAO activators. In some embodiments, the tissue is selected from the group consisting of a muscle tissue, a liver tissue, a heart tissue, a skin tissue and a hair follicle. In some embodiments, the tissue is a muscle tissue. In some embodiments, the tissuegenic cells are myogenic cells. In some embodiments, the myogenic cells are myoblasts and/or myocytes. In some embodiments, the tissue (e.g., muscle tissue) is contacted with the one or more FAO activators for no more than about 72 hours, no more than about 48 hours, or no more than about 24 hours.

One aspect of the present application provides a method of inducing proliferation of stem cells or tissuegenic cells in a tissue (e.g., muscle tissue), comprising contacting the tissue with one or more FAO activators. In some embodiments, the tissue has been injured. In some embodiments, the tissue has not undergone injury. In some embodiments, the tissue is contacted with the one or more FAO activators for no more than about 72 hours, no more than about 48 hours, or no more than about 24 hours. In some embodiments, the contacting is in vitro, ex vivo or in vivo. In some embodiments, the tissue is selected from the group consisting of a muscle tissue, a liver tissue, a heart tissue, a skin tissue and a hair follicle. In some embodiments, the tissue is a muscle tissue. In some embodiments, the tissuegenic cells are myogenic cells. In some embodiments, the myogenic cells are myoblasts and/or myocytes.

In some embodiments according to any one of the methods described above, the tissue is from an aged individual, e.g., a human individual of at least about any one of 50, 60, 70, 80, or more years old.

In some embodiments according to any one of the methods described above, the tissue is an injured tissue. In some embodiments, the tissue has not undergone injury.

One aspect of the present application provides a method of treating a disease or condition associated with a tissue (e.g., muscle tissue) in an individual, comprising administering an effective amount of a pharmaceutical composition comprising tissuegenic cells (e.g., myogenic cells) to the tissue of the individual, wherein the tissuegenic cells are contacted with one or more FAO activators prior to the administration of the pharmaceutical composition. In some embodiments, the method comprises contacting the tissuegenic cells with the one or more FAO activators prior to the administration of the pharmaceutical composition. In some embodiments, the tissuegenic cells are contacted with the one or more FAO activators for no more than about 72 hours, no more than about 48 hours, or no more than about 24 hours. In some embodiments, the tissuegenic cells are autologous. In some embodiments, the tissuegenic cells are allogenic. In some embodiments, the tissue is selected from the group consisting of a muscle tissue, a liver tissue, a heart tissue, a skin tissue and a hair follicle. In some embodiments, the tissue is a muscle tissue. In some embodiments, the tissuegenic cells are myogenic cells. In some embodiments, the myogenic cells are myoblasts and/or myocytes. In some embodiments, the pharmaceutical composition is administered intramuscularly. In some embodiments, the pharmaceutical composition is administered subcutaneously.

One aspect of the present application provides a method of treating a disease or condition associated with a tissue (e.g., muscle tissue) in an individual, comprising administering an effective amount of a pharmaceutical composition comprising one or more FAO activators to the individual. In some embodiments, the pharmaceutical composition is administered to the individual once every 24 hours, every 48 hours, or every 72 hours. In some embodiments, the pharmaceutical composition is administered to the tissue (e.g., muscle tissue) of the individual. In some embodiments, the pharmaceutical composition is administered to the individual systemically, such as orally. In some embodiments, the tissue is selected from the group consisting of a muscle tissue, a liver tissue, a heart tissue, a skin tissue and a hair follicle. In some embodiments, the tissue is a muscle tissue. In some embodiments, the pharmaceutical composition is administered intramuscularly. In some embodiments, the pharmaceutical composition is administered subcutaneously.

In some embodiments according to any one of the methods of treatment described above, the disease or condition is tissue injury. In some embodiments, the disease or condition is muscle injury. In some embodiments, the pharmaceutical composition is administered to the individual no more than about 72 hours, no more than about 48 hours, or no more than about 24 hours after the tissue injury.

In some embodiments according to any one of the methods of treatment described above, the disease or condition is tissue degeneration. In some embodiments, the disease or condition is muscle degeneration.

In some embodiments according to any one of the methods of treatment described above, the disease or condition is tissue fibrosis. In some embodiments, the disease or condition is muscle fibrosis.

In some embodiments according to any one of the methods of treatment described above, the disease or condition is aging.

In some embodiments according to any one of the methods of treatment described above, the disease or condition is selected from the group consisting of sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, and other genetically determined, environmentally determined or idiopathic disease processes causing loss or atrophy of tissue/organ/body part structure and function.

One aspect of the present application provides a method of providing one or more benefits of exercise and/or nutrition to a tissue (e.g., muscle tissue) of an individual, comprising administering an effective amount of a pharmaceutical composition comprising one or more FAO activators to the individual. In some embodiments, the pharmaceutical composition is administered to the individual once every 24 hours, every 48 hours, or every 72 hours. In some embodiments, the tissue is selected from the group consisting of a muscle tissue, a liver tissue, a heart tissue, a skin tissue and a hair follicle. In some embodiments, the tissue is a muscle tissue. In some embodiments, the pharmaceutical composition is administered to the tissue (e.g., muscle tissue) of the individual. In some embodiments, the pharmaceutical composition is administered intramuscularly. In some embodiments, the pharmaceutical composition is administered subcutaneously. In some embodiments, the pharmaceutical composition is administered to the individual systemically, such as orally. In some embodiments, the tissue is an injured tissue. In some embodiments, the tissue has not undergone injury.

In some embodiments according to any one of the methods of treatment described above, the individual is an aged individual, e.g., a human individual of at least about any one of 50, 60, 70, 80, or more years old.

In some embodiments according to any one of the methods described above, the one or more FAO activators increases mitochondrial FAO in a myogenic cell. In some embodiments, the one or more FAO activators increases mitochondrial FAO in a myogenic cell. In some embodiments, the one or more FAO activators increases mitochondrial oxygen consumption in a myogenic cell. In some embodiments, the one or more FAO activators does not affect mitochondrial biogenesis in a myogenic cell. In some embodiments, the one or more FAO activators does not affect membrane potential of a myogenic cell. In some embodiments, the one or more FAO activators increases level(s) of Pax7, MyoD (e.g., MyoD1), Ki67, MyoG, Myh3, PPARγ, PPARα, and/or H3K9acin a myogenic cell.

In some embodiments according to any one of the methods described above, the one or more FAO activators is a single FAO activator. In some embodiments, the one or more FAO activators is a combination of two or more (e.g., 2) FAO activators.

In some embodiments according to any one of the methods described above, the one or more FAO activators comprises an activator of a gene in the FAO pathway or lipid metabolism pathway. In some embodiments, the one or more FAO activators comprises an activator of a gene selected from the group consisting of transcriptional regulators of lipid metabolism, fatty acid transporters, lipases, carnitine palmitoyl-transferases, carnitine acetylase, acyl-CoA dehydrogenases, hydroxyacyl-CoA dehydrogenases, and the mitochondrial electron transfer flavoproteins. In some embodiments, the one or more FAO activators comprises an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACAD1, ACAD2, ACAD3, ACAD4, ACAD5, ACAD6, ACAD7, ACAD8, ACAD9, ACAD10, ACAD11, MCAD, LCAD, VLCAD, HADHA, HADHB, ETFA and ETFB. In some embodiments, the one or more FAO activators comprises one or more activators of PPARγ.

One aspect of the present application provides a method of increasing FAO in a tissuegenic cell (e.g., myogenic cell), comprising contacting the tissuegenic cell with one or more activators of PPARγ for no more than about 72 hours (such as no more than about 48 hours or no more than about 24 hours). In some embodiments, the tissuegenic cell is a myogenic cell. In some embodiments, the myogenic cell is a myoblast. In some embodiments, the myogenic cell is a myocyte. In some embodiments, the contacting is in vitro, ex vivo or in vivo.

One aspect of the present application provides a method of activating PPARγ in a tissuegenic cell, comprising contacting the tissuegenic cell with a prostaglandin selected from the group consisting of prostaglandin 12 (PGI2), prostaglandin D2 (PGD2), analogues thereof, and salts, solvates, tautomers, and stereoisomers thereof. In some embodiments, the tissuegenic cell is a myogenic cell. In some embodiments, the myogenic cell is a myoblast. In some embodiments, the myogenic cell is a myocyte. In some embodiments, the contacting is in vitro, ex vivo or in vivo. In some embodiments, the prostaglandin is PGI2, or a salt, solvate, tautomer, or stereoisomer thereof. In some embodiments, the prostaglandin is treprostinil, or a salt, solvate, tautomer, or stereoisomer thereof. In some embodiments, the prostaglandin is PGD2, or a salt, solvate, tautomer, or stereoisomer thereof.

In some embodiments according to any one of the methods described above, the one or more FAO activators or activators of PPARγ comprises a PPARγ agonist. In some embodiments, the PPARγ agonist is a thiazolidinedione or derivative thereof, or a salt, solvate, tautomer, or stereoisomer thereof. In some embodiments, the PPARγ agonist is a compound of Formula (I):

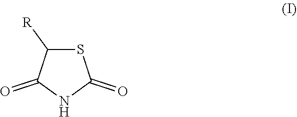

or a salt, solvate, tautomer, or stereoisomer thereof, wherein R is selected from the group consisting of hydrogen, unsubstituted and substituted C₁₋₆alkyl, unsubstituted and substituted C₂₋₆alkenyl, unsubstituted and substituted C₂₋₆ alkynyl, unsubstituted and substituted aryl, unsubstituted and substituted heteroaryl, and unsubstituted and substituted heterocyclyl. In some embodiments, the PPARγ agonist is a compound of Formula (II):

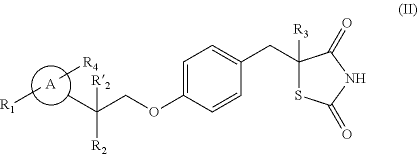

or a salt, solvate, tautomer, or stereoisomer thereof, wherein each of R₁ and R₄ is independently selected from the group consisting of hydrogen, halo, unsubstituted alkyl, alkyl substituted with 1-3 of halo, unsubstituted alkoxy, and alkoxy substituted with 1-3 of halo; wherein R₂ is selected from the group consisting of halo, hydroxy, unsubstituted and substituted alkyl; wherein R′₂ is hydrogen, or R₂ and R′₂ together form oxo; wherein R₃ is H; and wherein Ring A is a phenyl. In some embodiments, the PPARγ agonist is rosiglitazone, or a salt, solvate, tautomer, or stereoisomer thereof.

In some embodiments according to any one of the methods described above, the one or more FAO activators or activators of PPARγ comprises a prostaglandin selected from the group consisting of PGI2, PGD2, analogues thereof, and salts, solvates, tautomers, and stereoisomers thereof. In some embodiments, the prostaglandin is PGI2, or a salt, solvate, tautomer, or stereoisomer thereof. In some embodiments, the one or more FAO activators or activators of PPARγ are rosiglitazone and PGI2. In some embodiments, the prostaglandin is treprostinil, or a salt, solvate, tautomer, or stereoisomer thereof. In some embodiments, the one or more FAO activators comprises treprostinil, or a salt, solvate, tautomer, or stereoisomer thereof. In some embodiments, the one or more FAO activators or activators of PPARγ are rosiglitazone and treprostinil.

Further provided are pharmaceutical compositions, kits, and articles of manufacture for use in any one of the methods described above. In some embodiments, there is provided one or more FAO activators (e.g., a PPARγ agonist such as rosiglitazone, and/or PGI2, PGD2 or an analogue thereof) for use in any one of the methods described above. In some embodiments, there is provided use of one or more FAO activators (e.g., a PPARγ agonist such as rosiglitazone, and/or PGI2, PGD2 or an analogue thereof) in the preparation of a medicament for any one of the methods described above.

In some embodiments, there is provided a pharmaceutical composition comprising tissuegenic cells (e.g., myogenic cells), wherein the tissuegenic cells are contacted with a none or more FAO activators for no more than about 72 hours.

In some embodiments, there is provided a kit comprising a pharmaceutical composition comprising one or more FAO activators. In some embodiments, the kit comprises rosiglitazone and PGI2. In some embodiments, the kit comprises rosiglitazone and treprostinil.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIGS. 1A-1G show transient induction of fatty acid metabolism in human myocytes.

FIG. 1A shows clustergram heat map of intracellular metabolites in post-mitotic mononucleated human myocytes after differentiation for 48 h, relative to undifferentiated proliferative myoblasts. The results show that myocytes are very different from proliferative myoblasts in metabolism.

FIG. 1B shows relative abundance of metabolites that serve as hallmarks of myogenic differentiation, cyclic AMP, creatine and phosphocreatine.

FIG. 1C shows relative abundance of short chain acyl-carnitines, ranging from the 2-carbon (C2) acetyl-carnitine to the 6-carbon (C6) hexanoyl-carnitine.

FIG. 1D shows relative abundance of key glycolytic intermediates, glucose-6-phosphate (G6P) or fructose-6-phosphate (F6P), pyruvate and lactate.

FIG. 1E shows relative abundance of metabolites that regulate the redox balance, including the oxidized and reduced versions of glutathione and NAD⁺.

FIG. 1F shows relative mRNA expression levels of upstream regulators of fatty acid metabolism, over a 336 h time-course in human myoblast differentiation. The results show that nearly all upstream regulators of fatty acid metabolism genes rise transiently at 48 h.

FIG. 1G shows relative mRNA expression levels of downstream effectors of fatty acid metabolism, over a 336 h time-course in human myoblast differentiation. The results show that nearly all fatty acid metabolism genes rise transiently at 48 h.

FIGS. 2A-2F show transient induction of mitochondrial FAO in human myocytes.

FIG. 2A shows tracking of mitochondrial volume in post-mitotic mononucleated human myocytes after differentiation for 48 h, relative to undifferentiated proliferative myoblasts, by fluorescence staining with Mitotracker Red. The results show that myocytes have higher mitochondrial volume than proliferative myoblasts.

FIG. 2B shows quantification of mitochondrial volume per cell in post-mitotic mononucleated human myocytes after differentiation for 48 h, relative to undifferentiated proliferative myoblasts, by fluorescence staining with Mitotracker Red.

FIG. 2C shows tracking of mitochondrial membrane potential in post-mitotic mononucleated human myocytes after differentiation for 48 h, relative to undifferentiated proliferative myoblasts, by fluorescence staining with the JC1 dye.

FIG. 2D shows quantification of mitochondrial membrane potential per cell in post-mitotic mononucleated human myocytes after differentiation for 48 h, relative to undifferentiated proliferative myoblasts, by measuring the red:green fluorescence ratio of JC1.

FIG. 2E shows quantification of basal respiration rates in myocytes over the course of myogenic differentiation, by measuring basal oxygen consumption rates in fatty acid-supplemented differentiation media every 12 h for 84 h.

FIG. 2F shows quantification of maximal respiration rates in myocytes over the course of myogenic differentiation, by measuring maximal oxygen consumption rates in fatty acid-supplemented differentiation media after treatment with the proton gradient uncoupler FCCP every 12 h for 84 h.

FIGS. 3A-3G show that PPARγ drives the transient induction of mitochondrial FAO.

FIG. 3A shows relative mRNA expression levels of myogenic differentiation markers over the course of human myoblast differentiation for 84 h.

FIG. 3B shows relative mRNA expression level of MYOD1 over the course of human myoblast differentiation for 84 h.

FIG. 3C shows maximal respiration rates in myocytes after 48 h differentiation, following siRNA knockdown of MYOD1 (siMyod1), relative to a scrambled control siRNA.

FIG. 3D shows relative expression levels of various let-7 microRNAs over the course of human myoblast differentiation for 84 h.

FIG. 3E shows basal respiration rates in myocytes after 48 h differentiation, following knockdown with let-7 antagomir oligos (let-7 KD), or let-7 overexpression with duplex oligos (let-7 OE), relative to scrambled control oligos labeled with Cy5 or untransfected controls.

FIG. 3F shows relative mRNA expression levels of PPARα (dotted), PPARδ (dashed) and PPARγ (solid) over the course of human myoblast differentiation for 84 h. PPARγ rises transiently from 12 to 72 h, whereas PPARα rises steadily after 12 h.

FIG. 3G shows basal respiration rates in myocytes after inhibition with a PPARα and PPARγ inhibitor (iPPARα/γ) or a PPARδ inhibitor (iPPARδ) during different time-windows in myogenic differentiation.

FIGS. 4A-4E show transient mitochondrial FAO induction is necessary for normal myocyte differentiation.

FIG. 4A shows relative cell numbers after treating myocytes with the CPT1 inhibitor etomoxir during different time-windows in myogenic differentiation.

FIG. 4B shows a Western blot of the differentiation markers myogenin (MYOG) and myosin heavy chain (MHC) in myocytes after treatment with the CPT1 inhibitor etomoxir during different time-windows in myogenic differentiation.

FIG. 4C shows quantification of myosin heavy chain (MHC) protein levels in myocytes after treatment with the CPT1 inhibitor etomoxir during different time-windows in myogenic differentiation.

FIG. 4D shows quantification of myogenin (MYOG) protein levels in myocytes after treatment with the CPT1 inhibitor etomoxir during different time-windows in myogenic differentiation.

FIG. 4E shows quantification of myogenin (MYOG) protein levels in myocytes after treatment with the CPT1 inhibitor etomoxir during different time-windows in myogenic differentiation.

FIGS. 5A-5I show that early PPARγ induction is sufficient to promote myocyte differentiation.

FIG. 5A shows relative mRNA expression levels of myogenin (MYOG) after treatment with the PPARγ agonist rosiglitazone during different time-windows in myogenic differentiation at low-density conditions.

FIG. 5B shows relative mRNA expression levels of adult slow-twitch myosin heavy chain (MYH7) after treatment with the PPARγ agonist rosiglitazone during different time-windows in myogenic differentiation at low-density conditions.

FIG. 5C shows relative mRNA expression levels of perinatal myosin heavy chain (MYH8) after treatment with the PPARγ agonist rosiglitazone during different time-windows in myogenic differentiation at low-density conditions.

FIG. 5D shows immunofluorescence staining for the differentiation markers myosin heavy chain protein (MHC; purple), α-actinin (red) and nuclear myogenin (green) proteins, after treatment with the PPARγ agonist rosiglitazone (Rosi) during different time-windows in myogenic differentiation at low density conditions. The results show that rosiglitazone induces larger myocytes and myotubes with higher expression levels of MHC and α-actinin.

FIG. 5E shows quantification of myosin heavy chain (MHC) protein expression after treatment with the PPARγ agonist rosiglitazone (Rosi) during different time-windows in myogenic differentiation at low-density conditions.

FIG. 5F shows immunofluorescence staining for the differentiation markers myosin heavy chain protein (MHC; purple), α-actinin (red) and nuclear myogenin (green) proteins, after treatment with the PPARγ agonist rosiglitazone (Rosi) at the early 0-24 h time-window in myogenic differentiation at high density conditions. The results show that rosiglitazone induces myofibers with wider diameter and higher expression of MHC and α-actinin.

FIG. 5G shows a Western blot of myogenin (MYOG) and myosin heavy chain (MHC) protein expression after treatment with the PPARγ agonist rosiglitazone (Rosi) at the early 0-24 h time-window in myogenic differentiation at high density conditions.

FIG. 5H shows quantification of myogenin (MYOG) protein expression after treatment with the PPARγ agonist rosiglitazone (Rosi) at the early 0-24 h time-window in myogenic differentiation at high-density conditions.

FIG. 51 shows quantification of myosin heavy chain (MHC) protein expression after treatment with the PPARγ agonist rosiglitazone (Rosi) at the early 0-24 h time-window in myogenic differentiation at high-density conditions.

FIGS. 6A-6J show that early mitochondrial FAO induction promotes skeletal muscle regeneration in vivo.

FIG. 6A shows a schematic for cryoinjury of the tibialis anterior (TA) muscle in mice, followed by intramuscular injection of a single bolus of the PPARγ agonist rosiglitazone (Rosi) at 0, 24 or 48 h after injury. TA muscles were harvested for analysis 4 days after injury.

FIG. 6B shows a Western blot for the mouse differentiation markers MyoD, MYOG, MHC, and α-actinin in the TA muscle (4 days post-injury) after injection of a single bolus of the PPARγ agonist rosiglitazone (Rosi) at 0, 24 or 48 h after injury, relative to a PBS vehicle control (Ctr).

FIG. 6C shows quantification of the differentiation markers 1. MyoD, 2. MHC, and 3. α-actinin in the TA muscle (4 days post-injury) after injection of a single bolus of the PPARγ agonist rosiglitazone (Rosi) at 0, 24 or 48 h after injury, relative to a PBS vehicle control (Ctr).

FIG. 6D shows quantification of the remaining necrotic area in the TA muscle (4 days post-injury) after injection of a single bolus of the PPARγ agonist rosiglitazone (Rosi) at 0, 24 or 48 h after injury.

FIG. 6E shows a schematic for cryoinjury of the tibialis anterior (TA) muscle in mice, followed by intramuscular injection of a single bolus of GFP+ human myocytes treated with the PPARγ agonist rosiglitazone (Rosi) or DMSO vehicle control, 24 h after injury. TA muscles were harvested for analysis 4 days after injury.

FIG. 6F shows quantification of differentiated MHC+ cells amongst the GFP+ human myocytes that engrafted into the cryoinjured TA muscle 4 days post-injury.

FIG. 6G shows representative images of MHC+ cells (purple) amongst the GFP+ human myocytes, treated with the PPARγ agonist rosiglitazone (Rosi) or DMSO vehicle control that engrafted into the cryoinjured TA muscle 4 days post-injury.

FIG. 6H shows a Western blot of myogenin (MYOG) and myosin heavy chain (MHC) proteins in human myocytes treated at the 0-24 h window of differentiation with the PPARγ agonist rosiglitazone (Rosi), or rosiglitazone and etomoxir (Rosi+Eto), relative to the DMSO vehicle control, after 84 h of differentiation.

FIG. 6I shows quantification of 1. myogenin (MYOG) and 2. myosin heavy chain (MHC) proteins in human myocytes treated at the 0-24 h window of differentiation with the PPARγ agonist rosiglitazone (Rosi), or rosiglitazone and etomoxir (Rosi+Eto), relative to the DMSO vehicle control, after 84 h of differentiation.

FIG. 6J shows a model summarizing the effects of PPARγ-FAO activity on the different phases of myogenesis.

FIG. 7 shows quantification of JC1 red and JC1 green signals in myocytes after treatment with the CPT1 inhibitor etomoxir during different time-windows in myogenic differentiation.

FIG. 8 shows quantification of mitochondrial DNA copy number in myocytes after treatment with the CPT1 inhibitor etomoxir during different time-windows in myogenic differentiation.

FIG. 9 shows basal O₂ consumption ratein myocytes after 48 h differentiation, following siRNA knockdown of MYOD1 (siMyod1), relative to a scrambled control siRNA.

FIG. 10 shows maximal basal O₂ consumption rates in myocytes after 48 h differentiation, following knockdown with let-7 antagomir oligos (let-7 KD), or let-7 overexpression with duplex oligos (let-7 OE), relative to scrambled control oligos labeled with Cy5 or untransfected controls.

FIG. 11 shows maximal O₂ consumption rates in myocytes after inhibition with a PPARα and PPARγ inhibitor (iPPARα/γ) or a PPARS inhibitor (iPPARδ) during different time-windows in myogenic differentiation.

FIGS. 12A-12B show that PPARγ protein transiently rises during the early phase of myocyte differentiation.

FIG. 12A shows quantification of myosin heavy chain (MHC) proteins in human myocytes during the 0-96 h window of myogenic differentiation, via Western blot and densitometry.

FIG. 12B shows quantification of PPARγ (PPARG) and GAPDH proteins in human myocytes during the 0-96 h window of differentiation, via Western blot densitometry. PPARG protein transiently rises during the 24-72 h window of differentiation.

FIGS. 13A-13C show that transient knockdown of PPARγ (PPARG) using a doxycycline-repressible TetOff-shRNA against PPARG (TetOff-shPPARG) led to a reduction in differentiation efficiency, i.e., PPARG is necessary for normal myogenic differentiation.

FIG. 13A shows that when doxycycline is withdrawn (-dox), TetOff-shPPARG is activated, thereby reducing PPARG and the myogenic markers of differentiation, MHC I, MHC IIa and MHC IIx proteins, as quantified by Western blot densitometry.

FIG. 13B shows that when doxycycline is withdrawn (-dox), TetOff-shPPARG is activated, thereby reducing the myogenic markers of differentiation, ACTA1, MYOG, MYH7 and MYH8 mRNAs, as quantified by qRT-PCR (* P<0.05).

FIGS. 14A-14B show that Pax7+ muscle stem cells accumulate during aging in mouse skeletal muscles, indicating that the regeneration defect in aged muscles is not due to a defect in stem cell proliferation but a defect in stem cell differentiation.

FIG. 14A shows by immunofluorescence microscopy images demonstrating that Pax7+ muscle stem cells (green), counterstained with DAPI (blue nuclei), are more frequent in skeletal muscles (TA) of aged and sarcopenic 2-year-old mice, compared to young 6-week-old mice. Arrows point to exemplary nuclei of Pax7+ muscle stem cells.

FIG. 14B shows the quantification of Pax7+ muscle stem cells in skeletal muscles (TA) of aged and sarcopenic 2-year-old mice, compared to young 6-week-old mice (* P<0.05).

FIGS. 15A-15D show that early activation of PPARγ and thus mitochondrial FAO promotes skeletal muscle regeneration and reduces muscle fibrosis after aging in old animals in vivo.

FIG. 15A shows a schematic for cryoinjury of the tibialis anterior (TA) muscle in mice, followed by intramuscular injection of a single bolus of the PPARγ agonist rosiglitazone (Rosi) at 0, 24 or 48 h after injury. TA muscles were needle-biopsied 6 days after injury and harvested for analysis 27 days after injury.

FIG. 15B shows representative Masson trichrome staining images of the TA muscles after cryoinjury of the tibialis anterior (TA) muscle in aged and sarcopenic 2-year old mice, followed by intramuscular injection of a single bolus of the PPARγ agonist rosiglitazone (Rosi) at 0, 24 or 48 h after injury, relative to DMSO vehicle control injection in both 6-week young and 2-year old mice.

FIG. 15C shows quantification of the fibrotic area in the TA muscle (27 days post-injury) after injection of a single bolus of the PPARγ agonist rosiglitazone (Rosi) at 0, 24 or 48 h after injury, relative to DMSO vehicle control injection in both 6-week young and 2-year old mice. The result shows that, while aged mice show increased muscle fibrosis compared to young mice (***P<0.001), the PPARγ agonist rosiglitazone can reverse the aged muscle fibrosis if injected early at 0 h after injury (^{###}P<0.001).

FIG. 15D shows quantification of the regenerative index (fraction of nuclei in embryonic MHC-positive myofibers) in the TA muscle (6 days post-injury) after injection of a single bolus of the PPARγ agonist (Rosi) at 0, 24 or 48 h after injury, relative to DMSO vehicle control injection in both 6-week young and 2-year old mice. The result shows that, while aged mice show decreased muscle regeneration compared to young mice (***P<0.001), the PPARγ agonist rosiglitazone can restore aged muscle regeneration if injected early at 0 h after injury (^{##}P<0.01).

FIG. 15E shows quantification of the grip strength (27 days post-injury) after injection of a single bolus of the PPARγ agonist (Rosi) at 0, 24 or 48 h after injury, relative to DMSO vehicle control injection in both 6-week young and 2-year old mice. The result shows that, while aged mice show decreased grip strength compared to young mice (**P<0.01), the PPARγ agonist rosiglitazone can partially restore grip strength if injected early at 0 h after injury (^{#}P<0.05).

FIGS. 16A-16B show that a single intramuscular bolus of the PPARγ agonistrosiglitazone (Rosi) induces mitochondrial FAO during muscle regeneration in old animals in vivo, without significant effects on aging-induced obesity and thus systemic insulin sensitivity.

FIG. 16A shows that 27 days after intramuscular injection of a single bolus of the PPARγ agonist rosiglitazone (Rosi), there were no significant changes in the body weight or aging-induced obesity of aged and sarcopenic 2-year-old mice, relative to DMSO or untreated controls.

FIG. 16B shows that intramuscular injection of a single bolus of the PPARγ agonistrosiglitazone (Rosi) at 0 h post-injury in 2-year-old aged mice led to an induction of various FAO intermediates called acyl-carnitines at day 6, relative to injection of DMSO vehicle control in 2-year old mice and 6-week young mice, as measured by LC-MS/MS (Waters Xevo-G2XS).

FIGS. 17A-17D show that only the prostaglandins PGI2 and PGD2 can promote tissue regeneration.

FIG. 17A shows quantification of the regenerative index (fraction of nuclei in embryonic MHC-positive myofibers) in the TA muscle (6.5 days post-injury) after injection of a single bolus of the prostaglandin PGI2, relative to DMSO vehicle control (Con). The result shows that PGI2 can significantly increase muscle regeneration (P<0.001).

FIG. 17B shows quantification of the regenerative index (fraction of nuclei in embryonic MHC-positive myofibers) in the TA muscle (6.5 days post-injury) after injection of a single bolus of the prostaglandin PGF1a, relative to DMSO vehicle (Con). The result shows that PGF1a can significantly decrease muscle regeneration (P<0.05).

FIG. 17C shows quantification of the regenerative index (fraction of nuclei in embryonic MHC-positive myofibers) in the TA muscle (6.5 days post-injury) after injection of a single bolus of the prostaglandin PGD2, relative to DMSO vehicle (Con). The result shows that PGD2 can slightly but significantly increase muscle regeneration (P<0.01).

FIG. 17D shows quantification of the regenerative index (fraction of nuclei in embryonic MHC-positive myofibers) in the TA muscle (6.5 days post-injury) after injection of a single bolus of the prostaglandin PGG1, relative to DMSO vehicle (Con). The result shows that PGG1 has no significant effect on muscle regeneration (P>0.05).

FIGS. 18A-18H show that PGI2 can increase PPARγ (PPARG)-positive cells and boost an intermediate stage of myoblast differentiation both during muscle regeneration in vivo and in pure myoblasts cultured in vitro.

FIG. 18A shows the abundance of cyclic adenosine monophosphate (cAMP) in a subset of skeletal muscle cells within the injured region (IR) or the non-injured region (NR) during muscle regeneration in vivo, 6 days after injection of a single bolus of the prostaglandin PGI2, relative to the DMSO vehicle control, as quantified by matrix-assisted laser desorption ionization-mass spectrometry imaging (MALDI-MSI, Bruker Daltonics) of the TA muscle after cryoinjury. While GPCR-driven cAMP production is often thought to be the downstream mechanism of PGI2 signalling (Narumiya et al., 1999, DOI: 10.1152/physrev.1999.79.4.1193), the results show that cAMP was significantly decreased after PGI2 injection (***P<0.001), thus excluding the possibility that PGI2 could be exerting its pro-regenerative effects via cAMP signalling to protein kinase A (PKA), and further supporting PGI2's mechanism via other targets.

FIG. 18B shows quantification of the percentage fraction of PPARG-positive cells (by immunofluorescence) in the TA muscle at 1-2 days post-freeze injury (FI) after injection of a single bolus of the prostaglandin PGI2, relative to DMSO vehicle control. The result shows that PGI2 can significantly increase PPARG-positive cells during muscle regeneration (*P<0.05, ***P<0.001).

FIG. 18C shows quantification of PPARA, PPARD, and PPARG mRNA expression (by qRT-PCR) in the injured TA muscle after injection of a single bolus of the prostaglandin PGI2, relative to DMSO vehicle control. The result shows that PGI2 can significantly increase PPARG mRNA expression during muscle regeneration (**P<0.01).

FIG. 18D shows quantification of Pax7, MyoD, MyoG, Myh3 mRNA expression (by qRT-PCR) in the injured TA muscle after injection of a single bolus of the prostaglandin PGI2, relative to DMSO vehicle control. The result shows that PGI2 can significantly increase both the muscle stem cell markers Pax7 and MyoD and the myocyte differentiation markers MyoG and Myh3 during muscle regeneration (**P<0.01).

FIG. 18E shows quantification of PPARG, H3K9ac (acetylated histone H3 lysine 9) and MyoD protein expression by Western blot and densitometry in pure human myoblasts after treatment with the prostaglandin PGI2, the PGI2 analogue treprostinil, and the PPARG agonist rosiglitazone (Rosig), relative to DMSO vehicle control (Ctr). The result shows that PGI2 signalling increases PPARG protein, and thus histone H3 acetylation and MyoD protein to activate stem cells into myoblasts.

FIG. 18F shows quantification of a variety of myogenesis markers (by qRT-PCR) in pure human myoblasts after treatment with the prostaglandin PGI2, relative to DMSO vehicle control (Ctr). The result shows that PGI2 is sufficient to promote proliferative myoblasts to undergo differentiation (**P<0.01, ***P<0.001).

FIG. 18G shows quantification of a variety of myogenesis markers (by qRT-PCR) in pure human myocytes, 24 h after initiation of differentiation, after treatment with the prostaglandin PGI2, relative to DMSO vehicle control (Ctr). The result shows that PGI2 is sufficient to block committed myocytes from undergoing terminal differentiation (*P<0.05, **P<0.01, ***P<0.001).

FIG. 18H shows quantification of the PPARA, PPARD, PPARG, acetylated histone H3 lysine 9 (H3K9ac), PAX7, MyoD, MyoG and embryonic MHC (Myh3) protein expression by Western blot and densitometry in the non-injured region (NR) and injured region (IR) of TA muscle at 6 days post-cryoinjury after injection of a single bolus of the prostaglandin PGI2, relative to DMSO vehicle control. The result shows that PGI2 can increase PPARA, PPARD, PPARG, H3K9ac and all the myogenic markers including PAX7, MyoD, MyoG and Myh3 protein expression during muscle regeneration, in both the IR and the NR.

FIG. 18I shows quantification of PPARA, PPARD, PPARG and H3K9ac (acetylated histone H3 lysine 9) protein expression by Western blot and densitometry in the TA muscle 1-2 days post-cryoinjury after injection of a single bolus of the prostaglandin PGI2, relative to DMSO vehicle control. The result shows that PPARA, PPARD and PPARG proteins transiently increase during muscle regeneration (DMSO dl-2), but PGI2 accelerates the increase in PPARG, suppresses the increase in PPARA, and exerts little effect on PPARD (PGI2 dl-2). PGI2 induction of PPARG and mitochondrial FAO also increased protein acetylation and especially histone acetylation, as indicated by H3K9ac levels, as one of the mechanisms for promoting the intermediate stage of myoblast differentiation.

FIGS. 19A-19E show that PGI2 and PGI2 analogues can act synergistically with PPARG agonists to boost muscle regeneration in vivo.

FIG. 19A shows quantification of the percentage fraction of committed myoblasts (MyoG-positive Ki67-positive cells and MyoG-positive cells by immunofluorescence) in the TA muscle over 6 days post-injury after injection of a single bolus of the prostaglandin PGI2, relative to DMSO vehicle control. The result shows that PGI2 can significantly increase committed myoblasts during muscle regeneration (**P<0.01).

FIG. 19B shows quantification of the regenerative index (fraction of nuclei in embryonic MHC-positive myofibers) in the TA muscle (6.5 days post-injury) after injection of a single bolus of the prostaglandin PGI2 at different concentrations, relative to rosiglitazone (Rosi) alone. The results show that 6.5-13 mM PGI2 is the optimal concentration for muscle regeneration.

FIG. 19C shows quantification of the regenerative index (fraction of nuclei in embryonic MHC-positive myofibers) in the TA muscle (6.5 days post-injury) after injection of a single bolus of rosiglitazone (Rosi) at different concentrations. The results show that 0.5 mg/ulrosiglitazone is the optimal concentration for muscle regeneration.

FIG. 19D shows semi-quantification of the myogenic markers Pax7, MyoD, MyoG, and embryonic MHC Myh3 protein expression by Western blotdensitometry in the TA muscle at 6 days post-cryoinjuryafter injection of a single bolus of the prostaglandin PGI2 analogue treprostinil (TP) at day 0, followed by a single bolus of the PPARG agonist rosiglitazone (Rog) at day 1, relative to DMSO vehicle controls. The results show that PGI2 signalling alone can significantly increase Pax7, MyoD, MyoG and Myh3 protein expression during muscle regeneration, but the PGI2 analogue combined with rosiglitazone can enhance myogenesis markers even more.

FIG. 19E shows quantification of the regenerative index (fraction of nuclei in embryonic MHC-positive myofibers) in the TA muscle (6.5 days post-injury) after injection of a single bolus of the prostaglandin PGI2 at day 0, relative to DMSO vehicle at day 0, followed by a single bolus of the PPARG agonist rosiglitazone (Rosi) at day 1, relative to DMSO vehicle at day 1. The results show that both PGI2 alone and rosiglitazone alone can significantly increase muscle regeneration (***P<0.001, *P<0.05), but PGI2 combined with rosiglitazone can synergistically enhance muscle regeneration even more (***P<0.001, ^{##}P<0.01, ^{###}P<0.001).

FIG. 19F shows quantification of the regenerative index (fraction of nuclei in embryonic MHC-positive myofibers) in the TA muscle (6.5 days post-injury) after injection of a single bolus of the prostaglandin PGI2 analogue treprostinil (TP) at day 0, relative to DMSO vehicle at day 0, followed by a single bolus of the PPARG agonist rosiglitazone (Rosi) at day 1, relative to DMSO vehicle at day 1. The results show that rosiglitazone alone, TP alone, PGI2 alone, all can significantly increase muscle regeneration (***P<0.001), but TP combined with rosiglitazone can synergistically enhance muscle regeneration even more (***P<0.001).

FIG. 19G shows the relative distribution of the myofiber cross-sectional Feret diameters in the TA muscle (6.5 days post-injury) after injection of a single bolus of the prostaglandin PGI2 at day 0, relative to DMSO vehicle at day 0, followed by a single bolus of the PPARG agonist rosiglitazone (Rosi) at day 1, relative to DMSO vehicle at day 1, for both the injured region (IR) and the non-injured region (NR). The P-values from Kruskal-Wallis test for significant differences in the myofiber cross-sectional area distributions of each treatment category are shown below. The results show that either PGI2 alone or rosiglitazone alone can increase hypertrophic growth, but PGI2 combined with rosiglitazone can synergistically increase hypertrophic growth even more.

FIG. 19H shows the relative distribution of the myofiber cross-sectional Feret diameters in the TA muscle (6.5 days post-injury) after injection of a single bolus of the PGI2 analogue treprostinil (TP) at day 0, relative to DMSO vehicle at day 0, followed by a single bolus of the PPARG agonist rosiglitazone (Rosi) at day 1, relative to DMSO vehicle at day 1. The P-values from Kruskal-Wallis test for significant differences in the myofiber cross-sectional area distributions of each treatment category are shown next to the legend. The results show that either PGI2 analogue (TP) alone or rosiglitazone alone can increase hypertrophic growth, but PGI2 analogue (TP) combined with rosiglitazone can synergistically increase hypertrophic growth even more.

FIG. 19I shows quantification of the grip strength (14 days post-injury) after injection of a single bolus of the prostaglandin PGI2 or the PGI2 analogue treprostinil (TP) or DMSO vehicle control at 0 h post-injury, followed by a single bolus of the PPARG agonist (Rosi) or DMSO vehicle control at 24 h post-injury, in 6-week old mice after cryoinjury, relative to uninjured mice. The result shows that, while injured mice show decreased grip strength compared to uninjured mice, the PPARG agonist rosiglitazone (Rosi), the prostaglandin PGI2 or the PGI2 analogue treprostinil (TP) alone can all partially restore grip strength (*P<0.05). The results further show that PGI2 or treprostinil (TP) combined with rosiglitazone (Rosi) can further enhance the post-injury recovery of grip strength in synergy (**P<0.01).

FIGS. 20A-20B show that PGI2 signalling promotes the cell proliferation of pure primary human myoblasts cultured in vitro.

FIG. 20A shows that in early passage primary human myoblasts (passage 12), treatment with PGI2 significantly increased proliferation (***P<0.001).

FIG. 20B shows that in late passage primary human myoblasts (passage 18), treatment with PGI2 significantly increased proliferation (***P<0.001).

FIGS. 21A-21E show that PGI2 signalling activates muscle stem cell and progenitor cell proliferation in multiple muscle tissues even without injury, activating wound-less regeneration and reversing fibrosis during aging.

FIG. 21A shows that 2 days after intra-peritoneal injection of a single bolus of the PGI2 analogue treprostinil (TP), the fraction of proliferative muscle stem cells (Pax7-positive Ki67-positive cells by immunofluorescence), the total pool of muscle stem cells (Pax7-positive cells by immunofluorescence), and the total pool of proliferative cells (Ki67-positive cells by immunofluorescence) in the gastrocnemius muscle were all significantly increased (P<0.05), even without injury.

FIG. 21B shows that 2 days after intra-peritoneal injection of a single bolus of the PGI2 analogue treprostinil (TP), the fraction of proliferative muscle stem cells (Pax7-positive Ki67-positive cells by immunofluorescence), the total pool of muscle stem cells (Pax7-positive cells by immunofluorescence), and the total pool of proliferative cells (Ki67-positive cells by immunofluorescence) in the quadriceps muscles were all significantly increased (P<0.05), even without injury.

FIG. 21C shows that 2 days after intramuscular injection of a single bolus of PGI2 or the PGI2 analogue treprostinil (TP), the fraction of proliferative muscle stem cells (Pax7-positive Ki67-positive cells by immunofluorescence) in the TA muscle was significantly increased (***P<0.001), even without injury.

FIG. 21D shows quantification of relative changes in the % fibrotic area (Masson trichrome staining) in the TA muscle (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased muscle fibrosis, the PPARγ agonist rosiglitazone, PGI2, and treprostinil can all partially reverse the aged muscle fibrosis. Furthermore, combinations of PGI2 or treprostinil with rosiglitazone can act synergistically to reverse aged muscle fibrosis even more (*P<0.05, **P<0.01).

FIG. 21E shows quantification of relative changes in the percentage fraction of fibrotic precursors (PGDFRA-positive and Ki67-positive by immunofluorescence) in the TA muscle (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased muscle fibrotic precursors, the PPARγ agonist rosiglitazone, PGI2, and treprostinil can all partially suppress the aged muscle fibrotic precursors. Furthermore, combinations of PGI2 or treprostinil with rosiglitazone can act synergistically to suppress aged muscle fibrotic precursors even more (*P<0.05).

FIGS. 22A-22D show that PGI2 signalling activates stem cell and progenitor cell proliferation in multiple non-skeletal muscle tissues even without injury, thus activating wound-less regeneration.

FIG. 22A shows that 2 days after intra-peritoneal injection of a single bolus of the PGI2 analogue treprostinil (TP), the total pool of proliferative progenitor cells (Ki67-positive cells by immunofluorescence) in the endoderm-derived liver tissue was significantly increased (**P<0.01), even without injury.

FIG. 22B shows that 2 days after intra-peritoneal injection of a single bolus of the PGI2 analogue treprostinil (TP), the total pool of proliferative progenitor cells (Ki67-positive cells by immunofluorescence) in the mesoderm-derived heart and cardiac muscle tissue was significantly increased (**P<0.01), even without injury.

FIG. 22C shows that 2 days after intra-peritoneal injection of a single bolus of the PGI2 analogue treprostinil (TP), the total pool of proliferative progenitor cells (Ki67-positive cells by immunofluorescence) in the neuroectoderm-derived skin tissue was significantly increased (***P<0.01), even without injury.

FIG. 22D shows that 2 days after intra-peritoneal injection of a single bolus of the PGI2 analogue treprostinil (TP), the total pool of proliferative progenitor cells (Ki67-positive cells by immunofluorescence) in the skin tissue's telogen hair follicles was significantly increased (**P<0.001), even without injury.

FIG. 23A-C show that PGI2 signalling synergizes with PPARG signalling to suppress fibrotic precursors in multiple non-skeletal muscle tissues during aging.

FIG. 23A shows quantification of relative changes in the percentage fraction of fibrotic precursors (PGDFRA-positive and Ki67-positive by immunofluorescence) in the endoderm-derived liver tissue (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased liver fibrotic precursors, the PPARγ agonist rosiglitazone, PGI2, and treprostinil can all partially suppress the aged liver fibrotic precursors. Furthermore, combinations of PGI2 or treprostinil with rosiglitazone can act synergistically to suppress aged liver fibrotic precursors even more (*P<0.05).

FIG. 23B shows quantification of relative changes in the percentage fraction of fibrotic precursors (PGDFRA-positive and Ki67-positive by immunofluorescence) in the neuroectoderm-derived skin tissue (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased skin fibrotic precursors, the PPARγ agonist rosiglitazone, PGI2, and treprostinil can all partially suppress the aged skin fibrotic precursors. Furthermore, combinations of PGI2 or treprostinil with rosiglitazone can act synergistically to suppress aged skin fibrotic precursors even more (**P<0.01).

FIG. 23C shows quantification of relative changes in the percentage fraction of fibrotic precursors (PGDFRA-positive and Ki67-positive by immunofluorescence) in the mesoderm-derived heart tissue (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased cardiac fibrotic precursors, the PPARγ agonist rosiglitazone, PGI2, and treprostinil can all partially suppress the aged cardiac fibrotic precursors. Furthermore, combinations of PGI2 or treprostinil with rosiglitazone can act synergistically to suppress aged cardiac fibrotic precursors even more (**P<0.01).

FIG. 24A-C show that PGI2 signalling synergizes with PPARG signalling to suppress fibrosis in multiple non-skeletal muscle tissues during aging.

FIG. 24A shows quantification of relative changes in the % fibrotic area (Masson trichrome staining) in the endoderm-derived liver tissue (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased liver fibrosis, the PPARγ agonist rosiglitazone, PGI2, and treprostinil can all partially reverse the aged liver fibrosis. Furthermore, combinations of PGI2 or treprostinil with rosiglitazone can act synergistically to reverse aged liver fibrosis even more (*P<0.05, **P<0.01).

FIG. 24B shows quantification of relative changes in the % fibrotic area (Masson trichrome staining) in the neuroectoderm-derived skin tissue (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased skin fibrosis, the PPARγ agonist rosiglitazone, PGI2, and treprostinil can all partially reverse the aged skin fibrosis. Furthermore, combinations of PGI2 or treprostinil with rosiglitazone can act synergistically to reverse aged skin fibrosis even more (**P<0.01).

FIG. 24C shows quantification of relative changes in the % fibrotic area (Masson trichrome staining) in the mesoderm-derived heart tissue (7 days post-injection) after daily injection of the PPARγ agonist rosiglitazone (Rosi), PGI2, the PGI2 analogue treprostinil (TP), PGI2 and Rosi, or TP and Rosi, relative to DMSO vehicle control injection in 2-year old mice. The results show that, while aged mice show increased cardiac fibrosis, the PPARγ agonist rosiglitazone, PGI2, and treprostinil can all partially reverse the aged cardiac fibrosis. Furthermore, combinations of PGI2 or treprostinil with rosiglitazone can act synergistically to reverse aged cardiac fibrosis even more (*P<0.05, **P<0.01).

FIGS. 25A-25B show the results of attempts to combine PGI2 and PGI2 analogues with hepatocyte growth factor (HGF) to activate muscle regeneration without injury.

FIG. 25A shows quantification of the percentage fraction of proliferative myoblasts (MyoD-positive Ki67-positive cells by immunofluorescence) in the TA muscle 2 days after intramuscular injection of a single bolus of the prostaglandin PGI2, relative to DMSO vehicle control, with or without hepatocyte growth factor (HGF). HGF has been previously shown to activate muscle stem cell proliferation even without injury (Tatsumi et al., 1998, DOI: 10.1006/dbio.1997.8803). These results show that PGI2 with or without HGF significantly decreased (*P<0.05, **P<0.01), while HGF alone and treprostinil (TP) alone slightly increased proliferative myoblasts, thus excluding synergism between HGF and PGI2 signalling.

FIG. 25B shows quantification of the percentage fraction of proliferative muscle stem cells (Pax7-positive Ki67-positive cells by immunofluorescence) in the TA muscle 2 days after intramuscular injection of a single bolus of the prostaglandin PGI2, relative to DMSO vehicle control, with or without hepatocyte growth factor (HGF). HGF has been previously shown to activate muscle stem cell proliferation even without injury (Tatsumi et al., 1998; DOI: 10.1006/dbio.1997.8803). Our results show that PGI2 alone, HGF alone and the PGI2 analogue treprostinil (TP) alone significantly increased (***P<0.001), while HGF combined with PGI2 failed to increase proliferative muscle stem cells, thus excluding synergism between HGF and PGI2 signalling.

FIGS. 26A-26B show the results of attempts to combine PGI2 and PGI2 analogues with PPARD drugs to activate muscle regeneration without injury.

FIG. 26A shows quantification of the percentage fraction of proliferative muscle stem cells (Pax7-positive Ki67-positive cells by immunofluorescence) in the gastrocnemius muscle 2 days after intramuscular injection of a single bolus of the prostaglandin PGI2 analogue treprostinil (TP), with or without the PPARD inhibitor GSK3787 (GSK), relative to DMSO vehicle control and the PPARD agonist GW0742 (GW). PPARD has been shown to be a target of PGI2 in vascular cells (He et al., 2008, DOI: 10.1161/CIRCRESAHA.108.176057; Li et al., 2011, DOI: 10.1165/rcmb.2010-04280C). Our results show that the PGI2 analogue (TP) alone significantly increased (*P<0.05), while the PPARD agonist GW0742 surprisingly decreased (**P<0.01) proliferative muscle stem cells. The PPARD inhibitor GSK3787 alone had no effect, but specifically ablated the stimulatory effect of TP when co-treated, suggesting that PPARD is partially necessary but not sufficient to drive the stem cell activation effect of PGI2 and its analogues.

FIG. 26B shows quantification of the percentage fraction of proliferative myoblasts (MyoD-positive Ki67-positive cells by immunofluorescence) in the gastrocnemius muscle 2 days after intramuscular injection of a single bolus of the prostaglandin PGI2 analogue treprostinil (TP), with or without the PPARD inhibitor GSK3787 (GSK), relative to DMSO vehicle control and the PPARD agonist GW0742 (GW). PPARD agonists have been previously shown to be exercise mimetic drugs (Narkar et al., 2008; DOI: 10.1016/j.cell.2008.06.051). Our results surprisingly show that the PGI2 analogue (TP) alone slightly increased proliferative myoblasts, with or without PPARD inhibition by GSK3787. The PPARD agonist GW0742 alone had no effect, but the PPARD inhibitor GSK3787 alone slightly increased proliferative myoblasts, suggesting that PPARD is neither necessary nor sufficient to drive the stem cell activation effect of PGI2 and its analogues, but exerts complex feedback effects if inhibited.

FIG. 27A shows representative immunostaining images for embryonic MHC (MYH3) in the tibialis anterior (TA) muscle (6.5 days post-cryoinjury) after intramuscular injection of a single bolus of the PPARD agonist GW0742 or the PPARD inhibitor GSK3787, relative to DMSO vehicle control injection.

FIG. 27B shows quantification of the regenerative index (fraction of nuclei in embryonic MHC-positive myofibers) in the TA muscle (6.5 days post-injury) after injection of a single bolus of the PPARD agonist GW0742 or the PPARD inhibitor GSK3787, relative to DMSO vehicle (Con). The result shows that GW0742 can significantly decrease muscle regeneration (P<0.05), but not GSK3787, suggesting that PPARD does not drive skeletal muscle regeneration.

FIGS. 28A-28B show that the PGI2 analogue treprostinil acts synergistically with PPARG but not PPARA to activate stem cell proliferation.

FIG. 28A shows quantification of the percentage fraction of proliferative myoblasts (MyoD-positive Ki67-positive cells by immunofluorescence) in the gastrocnemius muscle 2 days after intraperitoneal injection of a single bolus of the prostaglandin PGI2 analogue treprostinil (TP), with or without the PPAR agonist fenofibrate (FF) or the PPARA/G agonist WY-14643 (WY), relative to DMSO control. Our results show that the PGI2 analogue (TP) alone and WY-14643 alone significantly increased (**P<0.01) proliferative myoblasts. The PPARA agonistfenofibrate (FF) alone had no effect, but specifically ablated the stimulatory effect of TP when co-treated, suggesting that PPARA downregulation is necessary but insufficient to drive the stem cell activation effect of PGI2 and its analogues. In contrast, combined treatment of treprostinil (TP) with WY-14643 (WY) synergistically increased proliferative myoblasts even further (*P<0.05), indicating that PGI2 signalling synergizes with PPARG not PPARA to activate stem cell proliferation.

FIG. 28B shows quantification of the percentage fraction of myoblasts (MyoD-positive cells by immunofluorescence) in the gastrocnemius muscle 2 days after intraperitoneal injection of a single bolus of the prostaglandin PGI2 analogue treprostinil (TP), with or without the PPARA agonist fenofibrate (FF) or the PPARA/G agonist WY-14643 (WY), relative to DMSO control. Our results show that the PGI2 analogue (TP) alone and WY-14643 alone significantly increased myoblasts (**P<0.01, *P<0.05). The PPARA agonist fenofibrate (FF) alone had no effect, but specifically ablated the stimulatory effect of TP when co-treated, suggesting that PPARA downregulation is necessary but insufficient to drive the stem cell activation effect of PGI2 and its analogues. In contrast, combined treatment of treprostinil (TP) with WY-14643 (WY) synergistically increased proliferative myoblasts even further, indicating that PGI2 signalling synergizes with PPARG not PPARA to activate stem cells.

### DETAILED DESCRIPTION

The present application provides compositions and methods of using activators of fatty acid oxidation (“FAO”) to promote tissue (e.g., muscle) regeneration in vitro or in vivo. The present application is at least in part based on the inventors' surprising discovery of a transient burst of FAO at early phases within 72 hours of primary human myoblast differentiation. Furthermore, the present application demonstrates that activation of FAO, e.g., by a PPARγ agonist (such as rosiglitazone) and/or a prostaglandin (such as prostaglandin 12 (PGI2), prostaglandin D2 (PGD2), or an analogue thereof), induces differentiation of myogenic cells (e.g., myoblasts or myocytes) in cell cultures and enhances myogenesis in an animal model of muscle injury. Notably, the PPARγ agonist rosiglitazone can enhance muscle regeneration in aged animals, e.g., in mice whose age is equivalent to 60-years old in humans. Furthermore, PGI2 and its analogues act upstream of PPARγ and can synergize with PPARγ agonists to enhance muscle regeneration after injury. PGI2 and analogues can also activate stem cells and regeneration in multiple muscle tissues and other organs (e.g., skin, liver, heart etc.) even without injury. The methods and compositions described herein can be used to induce differentiation and/or maturation of tissuegenic cells (e.g., myogenic cells), promote tissue growth (e.g., muscle growth), and treating diseases or conditions associated with a tissue (e.g., muscle) such as tissue injury, degeneration or aging.

Accordingly, in some embodiments, there is provided a method of promoting regeneration and/or growth of a tissue and/or inducing proliferation of stem cells, and/or inducing differentiation and/or maturation of tissuegenic cells in a tissue, comprising contacting the tissue with one or more FAO activators (e.g., PPARγ agonist such as rosiglitazone, and/or PGI2, PGD2 or an analogue thereof). In some embodiments, the tissue is contacted with the one or more FAO activators for no more than about 72 hours (e.g., no more than about 48 hours or no more than about 24 hours). In some embodiments, the tissue is a muscle tissue. In some embodiments, the tissuegenic cells are myogenic cells.

In some embodiments, there is provided a method of treating a disease or condition associated with a tissue in an individual, comprising administering an effective amount of a pharmaceutical composition comprising tissuegenic cells to the tissue of the individual, wherein the tissuegenic cells are contacted with one or more FAO activators (e.g., PPARγ agonist such as rosiglitazone, and/or PGI2, PGD2 or an analogue thereof) prior to the administration of the pharmaceutical composition. In some embodiments, the tissuegenic cells are contacted with the one or more FAO activators for no more than about 72 hours (e.g., no more than about 48 hours or no more than about 24 hours). In some embodiments, the tissue is a muscle tissue. In some embodiments, the tissuegenic cells are myogenic cells. In some embodiments, the disease or condition is tissue injury, tissue regeneration, tissue fibrosis or aging.

In some embodiments, there is provided a method of treating a disease or condition associated with a tissue in an individual, comprising administering an effective amount of a pharmaceutical composition comprising one or more FAO activators (e.g., PPARγ agonist such as rosiglitazone, and/or PGI2, PGD2 or an analogue thereof) to the individual. In some embodiments, the pharmaceutical composition is administered about once every 24 hours, 48 hours or 72 hours. In some embodiments, the disease or condition is tissue injury. In some embodiments, the pharmaceutical composition is administered to the individual no more than about 72 hours, no more than about 48 hours, or no more than about 24 hours after the tissue injury. In some embodiments, the tissue is a muscle tissue.

### I. Definitions

Terms are used herein as generally used in the art, unless otherwise defined as follows.

The terms “fatty acid oxidation” and “FAO” are used herein interchangeably to refer to the biochemical process of breaking down a fatty acid into acetyl-CoA units. In some embodiments, the FAO is in the mitochondria of a cell. In some embodiments, the FAO is in the peroxisome of a cell.

As used herein, “tissuegenic cells” refer to cells that can proliferate and/or differentiate to a specialized, mature cell type and to regenerate a tissue. Exemplary tissuegenic cells include but are not limited to stem cells, progenitor cells, precursor cells, and combinations thereof. As used herein, “myogenic cells” refer to cells that can proliferate and/or differentiate to give rise to a muscle tissue. Myogenic cells include, but are not limited to, muscle stem cells, myoblasts, myocytes, myotubes, and myofibers. The myogenic cells contemplated herein may give rise to skeletal muscle, smooth muscle, and/or cardiac muscle.

As used herein, a “stem cell” is an undifferentiated cell characterized by the ability of self-renewal through mitotic cell division and the potential to differentiate into progeny cells, including self-renewing progenitors, non-renewing progenitors, and terminally differentiated cells. “Muscle stem cells” refer to stem cells found in adult muscle tissues, including for example, satellite cells.

As used herein, “progenitor cells” refer to undifferentiated cells that have the potential to differentiate into specialized cell types in a tissue. Muscle progenitor cells include, but are not limited to, muscle stem cells and myoblasts. Primary adult muscle progenitor cells have limited proliferative capacities, upon which they enter a senescent state and lose both proliferative and differentiation capacities. In contrast, embryonic and fetal muscle progenitor cells have heightened proliferative capacities despite many rounds of mitosis, and manifest robust regenerative response upon injury and transplantation.

As used herein, “myoblasts” refer to mononuclear muscle progenitor cells that can differentiate to give rise to muscle cells.

As used herein, “myocytes” refer to mononuclear muscle cells that result from differentiation of muscle progenitor cells.

As used herein, “myotubes” refer to multi-nucleated muscle cells that result from the fusion of myocytes.

As used herein, “myofibers” refer to terminally differentiated, multi-nucleated, and striated muscle cells that develop from myotubes.

As used herein, “activator” refers to an agent that increases the activity, expression, and/or quantity of a target. The agent may be of any molecular entity, including but not limited to, small molecules, peptides, proteins, nucleic acids (e.g., RNA, DNA, microRNA, chemically modified nucleic acids, etc.), and combinations thereof. The target of an activator may be a gene, a small molecule (e.g., a metabolite), a protein, a molecular pathway, or any combination thereof. In some embodiments, an activator increases the activity, expression, and/or quantity of a target by about any one of at least 10%, 20%, 50%, 2×, 5×, 10×, 100×, 1000×, or more, including any value or range in between these values. An activator of a target may directly interact with (e.g., bind to) the target, or act in a signalling pathway upstream of the target to regulate the activity, expression and/or quantity of the target.

As used herein, “PPARγ agonist” refers to an agent that increases the activity, expression and/or quantity of PPARγ by binding to and activating PPARγ or a complex thereof. A PPARγ agonist may be of any suitable molecular entity, including small molecules, peptides, proteins, nucleic acids, and combinations thereof. In some embodiments, the PPARγ agonist mimics a natural ligand of PPARγ.

As used herein, “PPARγ,” “PPARg,” “PPARG,” or “PPAR-gamma” refers to peroxisome proliferator-activated receptor gamma, including all isoforms (PPARγ1-3) thereof. In some embodiments, PPARγ is PPARγ1. In some embodiments, PPARγ is PPARγ2. In some embodiments, PPARγ forms a complex with retinoid X receptor (RXR), which binds to specific regions on the DNA of target genes.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease or condition (e.g., preventing or delaying the worsening of the disease or condition), preventing or delaying the spread of the disease or condition, preventing or delaying the occurrence or recurrence of the disease or condition, delaying or slowing the progression of the disease or condition, ameliorating the disease state, providing a remission (whether partial or total) of the disease or condition, decreasing the dose of one or more other medications required to treat the disease or condition, delaying the progression of the disease or condition, increasing the quality of life, and/or prolonging survival. Also encompassed by “treatment” is a reduction of pathological consequence of the disease or condition. The methods of the present application contemplate any one or more of these aspects of treatment.

The terms “individual,” “subject” and “patient” are used interchangeably herein to describe a mammal, including humans. An individual includes, but is not limited to, human, bovine, ovine, porcine, equine, feline, canine, rodent, or primate. In some embodiments, the individual is human. In some embodiments, an individual suffers from a disease or condition. In some embodiments, the individual is in need of treatment. In some embodiments, the individual is an aged individual, e.g., a human individual of at least about any one of 50, 55, 60, 65, 70, 75, 80, 85 or more years old.

As is understood in the art, an “effective amount” refers to an amount of a composition (e.g., one or more FAO activators or myogenic cells) sufficient to produce a desired therapeutic outcome. For therapeutic use, beneficial or desired results include, e.g., decreasing one or more symptoms resulting from the disease or condition (biochemical, histologic and/or behavioral), including its complications and intermediate pathological phenotypes presented during development of the disease or condition, increasing the quality of life of those suffering from the disease or condition, decreasing the dose of other medications required to treat the disease or condition, enhancing effect of another medication, delaying the progression of the disease or condition, and/or prolonging survival of patients.

As used herein, the terms “cell” and “cell culture” are used interchangeably and all such designations include progeny. It is understood that all progeny may not be precisely identical in DNA content, due to deliberate or inadvertent mutations. Variant progeny that have the same function or biological activity as the original cells are included.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), neopentyl ((CH₃)₃CCH₂—), and n-hexyl (CH₃(CH₂)₅—).

“Alkylene” refers to divalent aliphatic hydrocarbylene groups preferably having from 1 to 10 and more preferably 1 to 3 carbon atoms that are either straight-chained or branched. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Alkenyl” refers to straight chain or branched hydrocarbyl groups having from 2 to 10 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

“Alkenylene” refers to straight chain or branched hydrocarbylene groups having from 2 to 10 carbon atoms and preferably 2 to 4 carbon atoms and having at least 1 and preferably from 1 to 2 sites of double bond unsaturation. Examples of alkenylene include, but is not limited to, vinylene (—CH═CH—), allylene (—CH₂C═C—), and but-3-en-1-ylene (—CH₂CH₂C═CH—). Included within this term are the cis and trans isomers or mixtures of these isomers.

“Alkynyl” refers to straight or branched hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Alkynylene” refers to straight or branched hydrocarbylene groups having from 2 to 6 carbon atoms and preferably 2 to 3 carbon atoms and having at least 1 and preferably from 1 to 2 sites of triple bond unsaturation. Examples of alkynylene include, but are not limited to, acetylenylene (—C≡C—), and propargylene (—CH₂C≡C—).

“Amino” refers to the group —NH₂.

“Substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aryl” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO— substituted alkyl, —SO-aryl, —SO-heteroaryl, —SO₂-alkyl, —SO₂-substituted alkyl, —SO₂-aryl, —SO₂— heteroaryl and trihalomethyl.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantyl, and the like.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuranyl, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO— heteroaryl, —SO₂-alkyl, —SO₂-substituted alkyl, —SO₂-aryl and —SO₂-heteroaryl, and trihalomethyl.

Examples of heteroaryls include, but are not limited to, pyrrole, imidazole, pyrazole, pyridine, pyrazine, pyrimidine, pyridazine, indolizine, isoindole, indole, purine, isoquinoline, quinoline, phthalazine, naphthyridine, quinoxaline, quinazoline, cinnoline, pteridine, carbazole, carboline, phenanthridine, acridine, phenanthroline, isothiazole, phenazine, isoxazole, phenoxazine, phenothiazine, piperidine, piperazine, phthalimide, 4,5,6,7-tetrahydrobenzo[b]thiophene, thiazole, thiophene, benzo[b]thiophene, and the like.

“Heterocycle,” “heterocyclic,” “heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂— moieties.

Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like.

Where a heteroaryl or heterocyclyl group is “substituted,” unless otherwise constrained by the definition for the heteroaryl or heterocyclic substituent, such heteroaryl or heterocyclic groups can be substituted with 1 to 5, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, azido, cyano, halogen, hydroxyl, oxo, thioketo, carboxyl, carboxyl ester, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, sulfonylamino, —SO-alkyl, —SO-substituted alkyl, —SO-aryl, —SO-heteroaryl, —SO— heterocyclyl, —SO₂-alkyl, —SO₂-substituted alkyl, —SO₂-aryl, —SO₂-heteroaryl, and —SO₂-heterocyclyl.

“Polyalkylene glycol” refers to straight or branched polyalkylene glycol polymers such as polyethylene glycol, polypropylene glycol, and polybutylene glycol. A polyalkylene glycol subunit is a single polyalkylene glycol unit. For example, an example of a polyethylene glycol subunit would be an ethylene glycol, —O—CH₂—CH₂—O—, or propylene glycol, —O—CH₂—CH₂—CH₂—O—, capped with a hydrogen at the chain termination point. Other examples of poly(alkylene glycol) include, but are not limited to, PEG, PEG derivatives such as methoxypoly(ethylene glycol) (mPEG), poly(ethylene oxide), PPG, poly(tetramethylene glycol), poly(ethylene oxide-co-propylene oxide), or copolymers and combinations thereof.

“Polyamine” refers to polymers having an amine functionality in the monomer unit, either incorporated into the backbone, as in polyalkyleneimines, or in a pendant group as in polyvinyl amines.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NRR, trihalomethyl, —CN, —OCN, —SCN, —NO, —NO₂, ═N₂, —N₃, —S(O)R⁷⁰, —S(O)₂R⁷⁰, —SO₃N, —S(O)₂OR⁷⁰, —OS(O)₂R⁷⁰, —OSO₃⁻M⁺, —OS(O)₂OR⁷⁰, —PO₃²⁻(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heterocycloalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 3-, 4-, 5-, 6-, or 7-membered heterocycloalkyl which may optionally include from 1 to 4 of the same or different additional heteroatoms selected from the group consisting of O, N and S, of which N may have —H, C₁-C₄ alkyl, .C(O)C₁₋₄alkyl, .CO₂C₁₋₄alkyl, or —S(O)₂C₁₋₄alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the embodiments and the other a typical counter ion such as chloride, or two ionized compounds disclosed herein can serve as counter ions for such divalent alkali earth ions, or a doubly ionized compound of the embodiments can serve as the counter ion for such divalent alkali earth ions).

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)R⁷⁰, —S(O)₂R⁷⁰, —SO₃⁻M⁺, —SO₃R⁷⁰, —OS(O)₂R⁷⁰, —OSO₃⁻M⁺, —OSO₃R⁷⁰, —PO₃²⁻(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the substituent groups disclosed with respect to the individual terms herein, substituent groups for hydrogens on nitrogen atoms in “substituted” heterocycloalkyl and cycloalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)R⁷⁰, —S(O)₂R⁷⁰, —S(O)₂O⁺M⁺, —S(O)₂OR⁷⁰, —OS(O)₂R⁷⁰, —OS(O)₂O⁺M⁺, —OS(O)₂OR⁷⁰, —PO₃²⁻(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or tautomer or stereoisomer thereof” is intended to include all permutations of salts, solvates, tautomers, and stereoisomers, such as a solvate of a pharmaceutically acceptable salt of a tautomer of a stereoisomer of subject compound.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “an,” and “the” include plural referents unless the context clearly dictates otherwise.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the FAO activators and methods of use thereof are specifically embraced by the present application and are disclosed herein just as if each and every combination were individually and explicitly disclosed herein.

### II. Methods of Tissue Regeneration

The present application provides methods of tissue (e.g., muscle) regeneration using one or more activators of fatty acid oxidation (“FAO activators”) in vitro or in vivo. The methods described herein can promote tissue regeneration both after injury and without injury (i.e., woundless tissue regeneration).

In some embodiments, there is provided a method of promoting regeneration of a tissue (e.g., muscle tissue), comprising contacting the tissue with an FAO activator. In some embodiments, the contacting is in vitro. In some embodiments, the contacting is ex vivo. In some embodiments, the contacting is in vivo. In some embodiments, the tissue is contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the tissue with two or more FAO activators. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting regeneration of a tissue (e.g., muscle tissue), comprising contacting the tissue with a PPARγ agonist. In some embodiments, the contacting is in vitro. In some embodiments, the contacting is ex vivo. In some embodiments, the contacting is in vivo. In some embodiments, the tissue is contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting regeneration of a tissue (e.g., muscle tissue), comprising contacting the tissue with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting regeneration of a tissue (e.g., muscle tissue), comprising contacting the tissue with a PPARγ agonist (e.g., rosiglitazone) and a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the PPARγ agonist and the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting regeneration of a muscle tissue, comprising contacting the muscle tissue with one or more FAO activators. In some embodiments, the contacting is in vitro. In some embodiments, the contacting is ex vivo. In some embodiments, the contacting is in vivo. In some embodiments, the muscle tissue is contacted with the FAO activator for no more than about 72 hours, no more than about 48 hours, or no more than about 24 hours. In some embodiments, the one or more FAO activators comprises an activator of a gene in the FAO pathway or lipid metabolism pathway. In some embodiments, the one or more FAO activators comprises one or more activators of PPARγ. In some embodiments, the one or more activators of PPARγ comprises a PPARγ agonist, such as rosiglitazone. In some embodiments, the one or more activators of PPARγ comprises a prostaglandin selected from the group consisting of PGI2, PGD2, and analogues thereof (e.g., treprostinil). In some embodiments, thione or more activators of PPARγ comprises rosiglitazone and PGI2, or rosiglitazone and treprostinil. In some embodiments, the muscle tissue is from an aged individual (e.g., a human individual of at least about 50 years old). In some embodiments, the muscle tissue is an injured tissue. In some embodiments, the muscle tissue has not undergone injury.

In some embodiments, there is provided a method of promoting growth of a tissue (e.g., muscle tissue), comprising contacting the tissue with an FAO activator. In some embodiments, the contacting is in vitro. In some embodiments, the contacting is ex vivo. In some embodiments, the contacting is in vivo. In some embodiments, the tissue is contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the tissue with two or more FAO activators. In some embodiments, the muscle tissue is from an aged individual (e.g., a human individual of at least about 50 years old). In some embodiments, the tissue is an injured tissue. In some embodiments, the tissue has not undergone injury.

In some embodiments, there is provided a method of promoting growth of a tissue (e.g., muscle tissue), comprising contacting the tissue with a PPARγ agonist. In some embodiments, the contacting is in vitro. In some embodiments, the contacting is ex vivo. In some embodiments, the contacting is in vivo. In some embodiments, the tissue is contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting growth of a tissue (e.g., muscle tissue), comprising contacting the tissue with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting growth of a tissue (e.g., muscle tissue), comprising contacting the tissue with a PPARγ agonist (e.g., rosiglitazone) and a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the PPARγ agonist and the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of promoting growth oaf muscle tissue, comprising contacting the muscle tissue with one or more FAO activators. In some embodiments, the contacting is in vitro. In some embodiments, the contacting is ex vivo. In some embodiments, the contacting is in vivo. In some embodiments, the muscle tissue is contacted with the FAO activator for no more than about 72 hours, no more than about 48 hours, or no more than about 24 hours. In some embodiments, the one or more FAO activators comprises an activator of a gene in the FAO pathway or lipid metabolism pathway. In some embodiments, the one or more FAO activators comprises one or more activators of PPARγ. In some embodiments, the one or more activators of PPARγ comprises a PPARγ agonist, such as rosiglitazone. In some embodiments, the one or more activators of PPARγ comprises a prostaglandin selected from the group consisting of PGI2, PGD2, and analogues thereof (e.g., treprostinil). In some embodiments, thione or more activators of PPARγ comprises rosiglitazone and PGI2, or rosiglitazone and treprostinil. In some embodiments, the muscle tissue is from an aged individual (e.g., a human individual of at least about 50 years old). In some embodiments, the muscle tissue is an injured tissue. In some embodiments, the muscle tissue has not undergone injury.

In some embodiments, there is provided a method of increasing expression of H3K9ac, Ki67, MyoD, MYOG, MYH7, and/or MYH8 in a tissuegenic cell (e.g., myogenic cell, such as myoblast or myocyte), comprising contacting the tissuegenic cell with an FAO activator. In some embodiments, the contacting is in vitro. In some embodiments, the contacting is ex vivo. In some embodiments, the contacting is in vivo. In some embodiments, the tissuegenic cell is contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CRAT, CPT1C, CPT2, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the tissuegenic cell with two or more FAO activators.

In some embodiments, there is provided a method of increasing expression of H3K9ac, Ki67, MyoD, MYOG, MYH7, and/or MYH8 in a tissuegenic cell (e.g., myogenic cell, such as myoblast or myocyte), comprising contacting the tissuegenic cell with a PPARγ agonist. In some embodiments, the contacting is in vitro. In some embodiments, the contacting is ex vivo. In some embodiments, the contacting is in vivo. In some embodiments, the tissuegenic cell is contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof.

In some embodiments, there is provided a method of increasing expression of H3K9ac, Ki67, MyoD, MYOG, MYH7, and/or MYH8 in a tissuegenic cell (e.g., myogenic cell, such as myoblast or myocyte), comprising contacting the tissuegenic cell with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissuegenic cell is contacted with the prostaglandin for no more than about 72 hours, no more than about 48 hours, or no more than about 24 hours.

In some embodiments, there is provided a method of increasing expression of H3K9ac, Ki67, MyoD, MYOG, MYH7, and/or MYH8 in a tissuegenic cell (e.g., myogenic cell, such as myoblast or myocyte), comprising contacting the tissuegenic cell with a PPARγ agonist (e.g., rosiglitazone) and a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissuegenic cell is contacted with the PPARγ agonist and the prostaglandin for no more than about 72 hours, no more than about 48 hours, or no more than about 24 hours.

In some embodiments, there is provided a method of promoting myogenesis in a muscle tissue, comprising contacting the muscle tissue with an FAO activator. In some embodiments, the contacting is in vitro. In some embodiments, the contacting is ex vivo. In some embodiments, the contacting is in vivo. In some embodiments, the muscle tissue is contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the muscle tissue with two or more FAO activators. In some embodiments, the muscle tissue is from an aged individual (e.g., a human individual of at least about 50 years old).

In some embodiments, there is provided a method of promoting myogenesis in a muscle tissue, comprising contacting the muscle tissue with a PPARγ agonist. In some embodiments, the contacting is in vitro. In some embodiments, the contacting is ex vivo. In some embodiments, the contacting is in vivo. In some embodiments, the muscle tissue is contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the muscle tissue is from an aged individual (e.g., a human individual of at least about 50 years old).

In some embodiments, there is provided a method of promoting myogenesis in a muscle tissue, comprising contacting the muscle tissue with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the muscle tissue is contacted with the prostaglandin for no more than about 72 hours, no more than about 48 hours, or no more than about 24 hours. In some embodiments, the muscle tissue is from an aged individual (e.g., a human individual of at least about 50 years old).

In some embodiments, there is provided a method of promoting myogenesis in a muscle tissue, comprising contacting the muscle tissue with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the muscle tissue is contacted with the PPARγ agonist and the prostaglandin for no more than about 72 hours, no more than about 48 hours, or no more than about 24 hours. In some embodiments, the muscle tissue is from an aged individual (e.g., a human individual of at least about 50 years old).

In some embodiments, there is provided a method of inducing differentiation and/or maturation of tissuegenic cells (e.g., myogenic cells, such as myoblasts and/or myocytes) in a tissue (e.g., muscle tissue), comprising contacting the tissue with an FAO activator. In some embodiments, the contacting is in vitro. In some embodiments, the contacting is ex vivo. In some embodiments, the contacting is in vivo. In some embodiments, the tissue is contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the tissue with two or more FAO activators. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing differentiation and/or maturation of tissuegenic cells (e.g., myogenic cells, such as myoblasts and/or myocytes) in a tissue (e.g., muscle tissue), comprising contacting the tissue with a PPARγ agonist. In some embodiments, the contacting is in vitro. In some embodiments, the contacting is ex vivo. In some embodiments, the contacting is in vivo. In some embodiments, the tissue is contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing differentiation and/or maturation of tissuegenic cells (e.g., myogenic cells, such as myoblasts and/or myocytes) in a tissue (e.g., muscle tissue), comprising contacting the tissue with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing differentiation and/or maturation of tissuegenic cells (e.g., myogenic cells, such as myoblasts and/or myocytes) in a tissue (e.g., muscle tissue), comprising contacting the tissue with a PPARγ agonist (e.g., rosiglitazone) and a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the PPARγ agonist and the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing differentiation and/or maturation of a myogenic cell (e.g., myoblast or myocyte) in a muscle tissue, comprising contacting the muscle tissue with one or more FAO activators. In some embodiments, the contacting is in vitro. In some embodiments, the contacting is ex vivo. In some embodiments, the contacting is in vivo. In some embodiments, the muscle tissue is contacted with the FAO activator for no more than about 72 hours, no more than about 48 hours, or no more than about 24 hours. In some embodiments, the one or more FAO activators comprises an activator of a gene in the FAO pathway or lipid metabolism pathway. In some embodiments, the one or more FAO activators comprises one or more activators of PPARγ. In some embodiments, the one or more activators of PPARγ comprises a PPARγ agonist, such as rosiglitazone. In some embodiments, the one or more activators of PPARγ comprises a prostaglandin selected from the group consisting of PGI2, PGD2, and analogues thereof (e.g., treprostinil). In some embodiments, the one or more activators of PPARγ comprises rosiglitazone and PGI2, or rosiglitazone and treprostinil. In some embodiments, the muscle tissue is from an aged individual (e.g., a human individual of at least about 50 years old). In some embodiments, the muscle tissue is an injured tissue. In some embodiments, the muscle tissue has not undergone injury.

In some embodiments, there is provided a method of inducing proliferation of stem cells (e.g., muscle stem cells) or tissuegenic cells (e.g., muscle progenitor cells) in a tissue (e.g., muscle tissue), comprising contacting the tissue with an FAO activator. In some embodiments, the contacting is in vitro. In some embodiments, the contacting is ex vivo. In some embodiments, the contacting is in vivo. In some embodiments, the tissue is contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the tissue with two or more FAO activators. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing proliferation of stem cells (e.g., muscle stem cells) or tissuegenic cells (e.g., muscle progenitor cells) in a tissue (e.g., muscle tissue), comprising contacting the tissue with a PPARγ agonist. In some embodiments, the contacting is in vitro. In some embodiments, the contacting is ex vivo. In some embodiments, the contacting is in vivo. In some embodiments, the tissue is contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing proliferation of stem cells (e.g., muscle stem cells) or tissuegenic cells (e.g., muscle progenitor cells) in a tissue (e.g., muscle tissue), comprising contacting the tissue with a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing proliferation of stem cells (e.g., muscle stem cells) or tissuegenic cells (e.g., muscle progenitor cells) in a tissue (e.g., muscle tissue), comprising contacting the tissue with a PPARγ agonist (e.g., rosiglitazone) and a prostaglandin selected from the group consisting of PGI2, PGD2 and analogues thereof (e.g., treprostinil). In some embodiments, the contacting is in vitro. In some embodiments, the contacting is ex vivo. In some embodiments, the contacting is in vivo. In some embodiments, the tissue is contacted with the PPARγ agonist and the prostaglandin for no more than about 72 hours, no more than about 48 hours, or no more than about 24 hours. In some embodiments, the tissue is from an aged individual (e.g., a human individual of at least about 50 years old). In some embodiments, the tissue is an injured tissue. In some embodiments, the tissue has not undergone injury.

In some embodiments, there is provided a method of inducing proliferation of muscle stem cells or myogenic cells (e.g., muscle progenitor cells) in a muscle tissue, comprising contacting the muscle tissue with one or more FAO activators. In some embodiments, the contacting is in vitro. In some embodiments, the contacting is ex vivo. In some embodiments, the contacting is in vivo. In some embodiments, the muscle tissue is contacted with the FAO activator for no more than about 72 hours, no more than about 48 hours, or no more than about 24 hours. In some embodiments, the one or more FAO activators comprises an activator of a gene in the FAO pathway or lipid metabolism pathway. In some embodiments, the one or more FAO activators comprises one or more activators of PPARγ. In some embodiments, the one or more activators of PPARγ comprises a PPARγ agonist, such as rosiglitazone. In some embodiments, the one or more activators of PPARγ comprises a prostaglandin selected from the group consisting of PGI2, PGD2, and analogues thereof (e.g., treprostinil). In some embodiments, the one or more activators of PPARγ comprises rosiglitazone and PGI2, or rosiglitazone and treprostinil. In some embodiments, the muscle tissue is from an aged individual (e.g., a human individual of at least about 50 years old). In some embodiments, the muscle tissue is an injured tissue. In some embodiments, the muscle tissue has not undergone injury.

Tissue regeneration, tissue growth, proliferation of stem cells and tissuegenic cells, and differentiation and maturation of tissuegenic cells may be assessed using known methods in the art. For example, muscle regeneration, muscle growth, myogenesis, proliferation of muscle stem cells, and differentiation and maturation of myogenic cells may be assessed by assessing cell morphology using microscopy (e.g., myotube thickness), or by assessing expression levels (e.g., mRNA and/or protein levels) of myogenic markers such as PAX7, MyoD (MYOD1), myogenin (MYOG), Myf5 (MYF5), MRF4 (MYF6), alpha actin 1 (ACTA1), alpha actinin 2 (ACTN2), adult type I myosin heavy chain (MYH7), adult type IIa myosin heavy chain (MYH2), adult type IIb myosin heavy chain (MYH4), adult type IIx myosin heavy chain (MYH1), embryonicmyosin heavy chain (MYH3), perinatal myosin heavy chain (MYH8), pan-myosin heavy chain (MHC), myosin light chain (MLC), and the troponins, or by assessing expression levels of proliferation markers such as Ki67.Protein expression levels may be determined by immunostaining or by Western blots. mRNA expression levels may be determined by quantitative reverse-transcription PCR, microarray, or next-generation sequencing.

In some embodiments, there is provided a method of increasing mitochondrial oxygen consumption in a tissuegenic cell (such as myogenic cell, e.g., myoblast or myocyte), comprising contacting the tissuegenic cell (such as myogenic cell, e.g., myoblast or myocyte) with an activator of PPARγ for no more than about 72 hours. In some embodiments, the activator of PPARγ is a PPARγ agonist. In some embodiments, the contacting is in vitro. In some embodiments, the contacting is ex vivo. In some embodiments, the contacting is in vivo. In some embodiments, the tissuegenic cell is contacted with the PPARγ agonist for no more than about 48 hours. In some embodiments, the tissuegenic cell is contacted with the PPARγ agonist for no more than about 24 hours. In some embodiments, the PPARγ agonist is rosiglitazone, or a salt, solvate, tautomer, or stereoisomer thereof. In some embodiments, the activator of PPARγ is a prostaglandin selected from the group consisting of PGI2, PGD2 and analogues thereof (e.g., treprostinil). In some embodiments, the method comprises contacting the myogenic cell with two or more activators of PPARγ, such as a PPARγ agonist (e.g., rosiglitazone) and a prostaglandin selected from the group consisting of PGI2, PGD2 and analogues thereof (e.g., treprostinil).

Mitochondrial oxygen consumption may be determined using any known methods in the art, for example, by Seahorse analysis. In some embodiments, the method increases maximal mitochondrial oxygen consumption. In some embodiments, the method increases basal mitochondrial oxygen consumption. In some embodiments, the method increases both maximal mitochondrial oxygen consumption and basal mitochondrial oxygen consumption. In some embodiments, the mitochondrial oxygen consumption increases by at least any one of 10%, 20%, 50%, 2×, 3×, 5×, 10× or more, including any value or range in between these values.

In some embodiments, there is provided a method of increasing FAO in a tissuegenic cell (such as myogenic cell, e.g., myoblast or myocyte), comprising contacting the tissuegenic cell (such as myogenic cell, e.g., myoblast or myocyte) with an activator of PPARγ for no more than about 72 hours. In some embodiments, the activator of PPARγ is a PPARγ agonist. In some embodiments, the contacting is in vitro. In some embodiments, the contacting is ex vivo. In some embodiments, the contacting is in vivo. In some embodiments, the tissuegenic cell is contacted with the PPARγ agonist for no more than about 48 hours. In some embodiments, the tissuegenic cell is contacted with the PPARγ agonist for no more than about 24 hours. In some embodiments, the PPARγ agonist is rosiglitazone, or a salt, solvate, tautomer, or stereoisomer thereof. In some embodiments, the activator of PPARγ is a prostaglandin selected from the group consisting of PGI2, PGD2 and analogues thereof (e.g., treprostinil). In some embodiments, the method comprises contacting the myogenic cell with two or more activators of PPARγ, such as a PPARγ agonist (e.g., rosiglitazone) and a prostaglandin selected from the group consisting of PGI2, PGD2 and analogues thereof (e.g., treprostinil).

The level of FAO may be determined using any known methods in the art, for example, by metabolomics and lipidomics analysis using mass spectrometry. In some embodiments, the level of FAO increases by at least any one of 10%, 20%, 50%, 2×, 3×, 5×, 10× or more, including any value or range in between these values.

The FAO activators (including activators of PPARγ, such as PPARγ agonists and prostaglandins) used in the methods described herein may have anyone or combination of features described in Section IV “fatty acid oxidation activators” below.

In some embodiments, the contacting of the tissue (e.g., muscle tissue) or tissuegenic cell (e.g., myogenic cell) with the one or more FAO activators (including activators of PPARγ, such as PPARγ agonists and prostaglandins) is transient. “Transient” used herein is no more than 72 hours, such as no more than about any one of 60 hours, 48 hours, 36 hours, 24 hours, 12 hours, or 6 hours, including any value or range in between these values. In some embodiments, the tissue (e.g., muscle tissue) or tissuegenic cell (e.g., myogenic cell) is contacted with the one or more FAO activators for no more than about 24 hours. In some embodiments, the tissue (e.g., muscle tissue) or tissuegenic cell (e.g., myogenic cell) is contacted with the one or more FAO activators for no more than about 48 hours.

In some embodiments, the tissue (e.g., muscle tissue) or tissuegenic cell (e.g., myogenic cell) is contacted with the one or more FAO activators at about any one of 0, 1, 2, 4, 6, 12, 18, 24, 36, 48, 60, or 72 hours, including any value or range in between these values, after the the tissue (e.g., muscle tissue) or tissuegenic cell (e.g., myogenic cell) is subject to a condition that induces tissue regeneration (e.g., myogenesis, such as inducing differentiation and/or maturation of myogenic cells). In some embodiments, the tissue (e.g., muscle tissue) or tissuegenic cell (e.g., myogenic cell) is contacted with the one or more FAO activators between about 0 hour and about 24 hours after the tissue (e.g., muscle tissue) or tissuegenic cell (e.g., myogenic cell) is subject to a condition that induces tissue regeneration (e.g., myogenesis, such as inducing differentiation and/or maturation of myogenic cells). In some embodiments, the tissue (e.g., muscle tissue) or tissuegenic cell (e.g., myogenic cell) is contacted with the one or more FAO activators between about 0 hour and about 48 hours after the tissue (e.g., muscle tissue) or tissuegenic cell (e.g., myogenic cell) is subject to a condition that induces tissue regeneration (e.g., myogenesis, such as inducing differentiation and/or maturation of myogenic cells). In some embodiments, the tissue (e.g., muscle tissue) or tissuegenic cell (e.g., myogenic cell) is contacted with the one or more FAO activators between about 24 hours and about 48 hours after the tissue (e.g., muscle tissue) or tissuegenic cell (e.g., myogenic cell) is subject to a condition that induces tissue regeneration (e.g., myogenesis, such as inducing differentiation and/or maturation of myogenic cells).

Exemplary conditions that induce tissue regeneration (such as myogenesis) include, for example, culturing in a differentiation medium such as in DMEM/F12 or DMEM medium, supplemented with about 2% KnockOut Serum Replacement or about 2% horse serum, and 1% L-glutamine.

In some embodiments, there is provided a method of increasing the activity of mitochondria fatty acid oxidation to promote early cellular differentiation in human myocytes.

In some embodiments, there is provided a method of increasing the mitochondrial oxygen consumption to promote early cellular differentiation in human myocytes.

In some embodiments, there is provided a method of increasing PPARγ activity to promote early cellular differentiation in human myocytes.

In some embodiments, transiently increasing the mitochondria fatty acid oxidation increases myogenic differentiation. In some embodiments, transiently increasing MyoD1 promotes myogenic differentiation. In some embodiments, transiently activating PPARγ promotes myogenic differentiation through increasing mitochondrial fatty acid oxidation transiently.

For example, rosiglitazone treatment of myocytes under an exemplary cell culture condition at the 0-24 hour time-window uniquely upregulated the mRNA levels of myogenin (MYOG), adult type I myosin heavy chain (MYH7) and perinatal myosin heavy chain (MYH8), whereas other time-windows of treatment had no significant effects at the end of 96 hours. Rosiglitazone treatment of myocytes at the 0-24 hour and 24-48 hour time windows in an exemplary culture condition can significantly enhance myogenesis. However, rosiglitazone suppresses myogenesis in the other time windows under the same conditions.

In some embodiments, contacting human myocytes seeded at high density with rosiglitazone results in more mature and hypertrophic human myotubes compared to the same culturing condition without rosiglitazone.

In some embodiments, there is provided a method of activating PPARγ in a tissuegenic cell (e.g., myogenic cell), comprising contacting the tissuegenic cell with a prostaglandin selected from the group consisting of prostaglandin 12 (PGI2), prostaglandin D2 (PGD2), analogues thereof, and salts, solvates, tautomers, and stereoisomers thereof. In some embodiments, the contacting is in vitro. In some embodiments, the contacting is ex vivo. In some embodiments, the contacting is in vivo. In some embodiments, the tissuegenic cell is contacted with the prostaglandin for no more than about 72 hours, no more than about 48 hours, or no more than about 24 hours. In some embodiments, the method further comprises contacting the tissuegenic cell with a PPARγ agonist (e.g., rosiglitazone). In some embodiments, the prostaglandin increases the PPARγ expression and/or activity by at least any one of 10%, 20%, 50%, 2×, 3×, 5×, 10× or more, including any value or range in between these values.

The methods described herein are applicable to tissues (e.g., muscle tissues) and tissuegenic cells (e.g., myogenic cells) from various organisms, such as human, non-human primate (e.g., cynomolgus monkey, rhesus monkey, etc.), mouse, rat, cat, dog, hamster, rabbit, pig, cow, goat, sheep, horse, donkey, deer, mammal, bird, reptile, amphibian, fish, arthropod, mollusk, echinoderm, cnidarian, nematode, annelid, platyhelminth, etc.

In some embodiments, the tissue (e.g., muscle tissue) is from an individual. In some embodiments, the tissue (e.g., muscle tissue) is obtained by in vitro cell culture. In some embodiments, the tissue (e.g., muscle tissue) is an injured tissue. In some embodiments, the tissue (e.g., muscle tissue) has not undergone injury. In some embodiments, the tissue (e.g., muscle tissue) is from an aged individual, such as a rodent of at least 1 years old, 1.5 years old, 2 years old, or more, or a human of at least about any one of 50, 55, 60, 65, 70, 75, 80, 85 or more years old.

The methods described herein are applicable for a variety of tissues, including, but are not limited to tissues derived from endoderm, mesoderm, or neuroectoderm. In some embodiments, the tissue is a connective tissue (for example, loose connective tissue, dense connective tissue, elastic tissue, reticular connective tissue and adipose tissue), a muscle tissue (for example, skeletal muscle, smooth muscle and cardiac muscle), urogenital tissue, gastrointestinal tissue, lung tissue, bone tissue, nerve tissue and epithelial tissue (for example, a single layer of epithelial and stratified epithelium). In some embodiments, the tissue is of an organ selected from the group consisting of heart, liver, kidney, lung, stomach, intestine, bladder, and brain. In some embodiments, the tissue is a liver tissue. In some embodiments, the tissue is a heart tissue. In some embodiments, the tissue is a skin tissue. In some embodiments, the tissue is a hair follicle. In some embodiments, the artificial tissue is a muscle tissue.

In some embodiments, the tissue is a skeletal muscle tissue. In some embodiments, the tissue is anon-skeletal muscle tissue. In some embodiments, the non-skeletal muscle tissue is a mesodermal tissue. In some embodiments, the non-skeletal muscle tissue is heart and cardiac muscle tissue. In some embodiments, the non-skeletal muscle tissue is an endodermal tissue. In some embodiments, the non-skeletal muscle tissue is liver tissue. In some embodiments, the non-skeletal muscle tissue is a neuroectodermal tissue. In some embodiments, the non-skeletal muscle tissue is skin tissue. In some embodiments, the non-skeletal muscle tissue is the hair follicles.

In some embodiments, the muscle tissue comprises myogenic cells, such as myoblasts and/or myocytes. In some embodiments, the muscle tissue comprises at least about any one of 0.1%, 0.5%, 1%, 2%, 5%, 10%, 20%, 30% or more myogenic cells, including any value or range in between these values.

In some embodiments, the myogenic cell is a myoblast. In some embodiments, the myogenic cell is a Pax7⁻ Pax3⁺ MyoD⁺ myogenin and/or Pax7⁺ Pax3⁻MyoD⁺ myogenin⁻ cell. In some embodiments, the myogenic cell is a myocyte. In some embodiments, the myocyte is a Pax3⁻Pax7⁻ MyoD⁺ myogenin⁺ cell. In some embodiments, the myogenic cell is a primary cell. In some embodiments, the myogenic cell is derived from a cell line. In some embodiments, the myogenic cell is not derived from a cell line. In some embodiments, the myogenic cell is not derived from an immortal cell line.

### III. Methods of Treatment

The present application further provides methods of treating a disease or condition associated with a tissue (e.g., muscle disease or condition) using one or more FAO activators. Any one of the methods of tissue regeneration described in Section II “methods of tissue regeneration” above may be used for treatment of a disease or condition associated with a tissue. The one or more FAO activators (including activators of PPARγ, such as PPARγ agonists and prostaglandins) used herein may have anyone or combination of features described in Section IV “fatty acid oxidation activators” below. Suitable diseases or conditions include, but are not limited to, sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, and other genetically determined, environmentally determined or idiopathic disease processes causing loss or atrophy of tissue/organ/body part structure and function.

Administration of FAO activators

In some embodiments, there is provided a method of treating a disease or condition associated with a tissue (e.g., a muscle disease or condition) in an individual, comprising administering an effective amount of a pharmaceutical composition comprising an FAO activator to the individual. In some embodiments, the disease or condition is tissue injury. In some embodiments, the disease or condition is tissue degeneration. In some embodiments, the disease or condition is tissue fibrosis. In some embodiments, the disease or condition is aging. In some embodiments, the tissue is selected from the group consisting of a muscle tissue, a liver tissue, a heart tissue, a skin tissue and a hair follicle. In some embodiments, the pharmaceutical composition is administered to the individual once every 24 hours, every 48 hours, or every 72 hours. In some embodiments, the pharmaceutical composition is administered to the tissue of the individual. In some embodiments, the pharmaceutical composition is administered to the individual systemically, e.g., orally. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the pharmaceutical composition comprises two or more FAO activators. In some embodiments, the individual is an aged individual (e.g., a human individual of at least about 50 years old). In some embodiments according to any one of the methods of treatment described above, the disease or condition is selected from the group consisting of sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, and other genetically determined, environmentally determined or idiopathic disease processes causing loss or atrophy of tissue/organ/body part structure and function.

In some embodiments, there is provided a method of treating a disease or condition associated with a tissue (e.g., a muscle disease or condition) in an individual, comprising administering an effective amount of a pharmaceutical composition comprising a PPARγ agonist to the individual. In some embodiments, the disease or condition is tissue injury. In some embodiments, the disease or condition is tissue degeneration. In some embodiments, the disease or condition is tissue fibrosis. In some embodiments, the disease or condition is aging. In some embodiments, the tissue is selected from the group consisting of a muscle tissue, a liver tissue, a heart tissue, a skin tissue and a hair follicle. In some embodiments, the pharmaceutical composition is administered to the individual once every 24 hours, every 48 hours, or every 72 hours. In some embodiments, the pharmaceutical composition is administered to the tissue of the individual. In some embodiments, the pharmaceutical composition is administered to the individual systemically, e.g., orally. In some embodiments, the PPARγ agonist is rosiglitazone, or a salt, solvate, tautomer, or stereoisomer thereof. In some embodiments, the individual is an aged individual (e.g., a human individual of at least about 50 years old). In some embodiments according to any one of the methods of treatment described above, the disease or condition is selected from the group consisting of sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, and other genetically determined, environmentally determined or idiopathic disease processes causing loss or atrophy of tissue/organ/body part structure and function.

In some embodiments, there is provided a method of treating a disease or condition associated with a tissue (e.g., a muscle disease or condition) in an individual, comprising administering an effective amount of a pharmaceutical composition comprising a prostaglandin to the individual, wherein the prostaglandin is selected from the group consisting of PGI2, PGD2 and analogues thereof (e.g., treprostinil). In some embodiments, the disease or condition is tissue injury. In some embodiments, the disease or condition is tissue degeneration. In some embodiments, the disease or condition is tissue fibrosis. In some embodiments, the disease or condition is aging. In some embodiments, the tissue is selected from the group consisting of a muscle tissue, a liver tissue, a heart tissue, a skin tissue and a hair follicle. In some embodiments, the pharmaceutical composition is administered to the individual once every 24 hours, every 48 hours, or every 72 hours. In some embodiments, the pharmaceutical composition is administered to the tissue of the individual. In some embodiments, the pharmaceutical composition is administered to the individual systemically, e.g., orally. In some embodiments, the individual is an aged individual (e.g., a human individual of at least about 50 years old). In some embodiments according to any one of the methods of treatment described above, the disease or condition is selected from the group consisting of sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, and other genetically determined, environmentally determined or idiopathic disease processes causing loss or atrophy of tissue/organ/body part structure and function.

In some embodiments, there is provided a method of treating a disease or condition associated with a tissue (e.g., a muscle disease or condition) in an individual, comprising administering an effective amount of a pharmaceutical composition comprising a PPARγ agonist (e.g., rosiglitazone) and a prostaglandin to the individual, wherein the prostaglandin is selected from the group consisting of PGI2, PGD2 and analogues thereof (e.g., treprostinil). In some embodiments, the disease or condition is tissue injury. In some embodiments, the disease or condition is tissue degeneration. In some embodiments, the disease or condition is tissue fibrosis. In some embodiments, the disease or condition is aging. In some embodiments, the tissue is selected from the group consisting of a muscle tissue, a liver tissue, a heart tissue, a skin tissue and a hair follicle. In some embodiments, the pharmaceutical composition is administered to the individual once every 24 hours, every 48 hours, or every 72 hours. In some embodiments, the pharmaceutical composition is administered to the tissue of the individual. In some embodiments, the pharmaceutical composition is administered to the individual systemically, e.g., orally. In some embodiments, the individual is an aged individual (e.g., a human individual of at least about 50 years old). In some embodiments, the pharmaceutical composition comprises rosiglitazone and PGI2. In some embodiments, the pharmaceutical composition comprises rosiglitazone and treprostinil. In some embodiments according to any one of the methods of treatment described above, the disease or condition is selected from the group consisting of sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, and other genetically determined, environmentally determined or idiopathic disease processes causing loss or atrophy of tissue/organ/body part structure and function.

In some embodiments, there is provided a method of treating a muscle disease or condition in an individual, comprising administering an effective amount of a pharmaceutical composition comprising one or more FAO activators to the individual. In some embodiments, the muscle disease or condition is muscle injury. In some embodiments, the muscle disease or condition is muscle degeneration. In some embodiments, the muscle disease or condition is muscle fibrosis. In some embodiments, the muscle disease or condition is aging. In some embodiments, the pharmaceutical composition is administered to the individual once every 24 hours, every 48 hours, or every 72 hours. In some embodiments, the pharmaceutical composition is administered to a muscle tissue of the individual, e.g., intramuscularly or subcutaneously. In some embodiments, the pharmaceutical composition is administered to the individual systemically, e.g., orally. In some embodiments, the individual is an aged individual (e.g., a human individual of at least about 50 years old). In some embodiments, the one or more FAO activators comprises an activator of a gene in the FAO pathway or lipid metabolism pathway. In some embodiments, the one or more FAO activators comprises one or more activators of PPARγ. In some embodiments, the one or more activators of PPARγ comprises a PPARγ agonist, such as rosiglitazone. In some embodiments, the one or more activators of PPARγ comprises a prostaglandin selected from the group consisting of PGI2, PGD2, and analogues thereof (e.g., treprostinil). In some embodiments, the one or more activators of PPARγ comprises rosiglitazone and PGI2, or rosiglitazone and treprostinil.

In some embodiments, there is provided a method of treating injury to a tissue (e.g., muscle tissue) in an individual, comprising administering an effective amount of a pharmaceutical composition comprising an FAO activator to the individual. In some embodiments, the tissue is selected from the group consisting of a muscle tissue, a liver tissue, a heart tissue, a skin tissue and a hair follicle. In some embodiments, the pharmaceutical composition is administered to the individual no more than about 72 hours, no more than about 48 hours, or no more than about 24 hours after the tissue injury. In some embodiments, the pharmaceutical composition is administered to the individual no more than about 24 hours after the injury. In some embodiments, the pharmaceutical composition is administered to the tissue of the individual. In some embodiments, the pharmaceutical composition is administered to the individual systemically, e.g., orally. In some embodiments, the FAO activator is an activator of a gene in the FAO pathway or lipid metabolism pathway. In some embodiments, the FAO activator is an activator of a gene selected from the group consisting of PPARα, PPARS, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the pharmaceutical composition comprises two or more FAO activators. In some embodiments, the individual is an aged individual (e.g., a human individual of at least about 50 years old).

In some embodiments, there is provided a method of treating injury to a tissue (e.g., muscle tissue) in an individual, comprising administering an effective amount of a pharmaceutical composition comprising a PPARγ agonist to the individual. In some embodiments, the tissue is selected from the group consisting of a muscle tissue, a liver tissue, a heart tissue, a skin tissue and a hair follicle. In some embodiments, the pharmaceutical composition is administered to the individual no more than about 72 hours, no more than about 48 hours, or no more than about 24 hours after the tissue injury. In some embodiments, the pharmaceutical composition is administered to the individual no more than about 24 hours after the injury. In some embodiments, the pharmaceutical composition is administered to the tissue of the individual. In some embodiments, the pharmaceutical composition is administered to the individual systemically, e.g., orally. In some embodiments, the PPARγ agonist is rosiglitazone, or a salt, solvate, tautomer, or stereoisomer thereof. In some embodiments, the individual is an aged individual (e.g., a human individual of at least about 50 years old).

In some embodiments, there is provided a method of treating injury to a tissue (e.g., muscle tissue) in an individual, comprising administering an effective amount of a pharmaceutical composition comprising a prostaglandin to the individual, wherein the prostaglandin is selected from the group consisting of PGI2, PGD2 and analogues thereof (e.g., treprostinil). In some embodiments, the tissue is selected from the group consisting of a muscle tissue, a liver tissue, a heart tissue, a skin tissue and a hair follicle. In some embodiments, the pharmaceutical composition is administered to the individual no more than about 72 hours, no more than about 48 hours, or no more than about 24 hours after the tissue injury. In some embodiments, the pharmaceutical composition is administered to the individual no more than about 24 hours after the injury. In some embodiments, the pharmaceutical composition is administered to the tissue of the individual. In some embodiments, the pharmaceutical composition is administered to the individual systemically, e.g., orally. In some embodiments, the individual is an aged individual (e.g., a human individual of at least about 50 years old).

In some embodiments, there is provided a method of treating injury to a tissue (e.g., muscle tissue) in an individual, comprising administering an effective amount of a pharmaceutical composition comprising a PPARγ agonist (e.g., rosiglitazone) and a prostaglandin to the individual, wherein the prostaglandin is selected from the group consisting of PGI2, PGD2 and analogues thereof (e.g., treprostinil). In some embodiments, the tissue is selected from the group consisting of a muscle tissue, a liver tissue, a heart tissue, a skin tissue and a hair follicle. In some embodiments, the pharmaceutical composition is administered to the individual no more than about 72 hours, no more than about 48 hours, or no more than about 24 hours after the tissue injury. In some embodiments, the pharmaceutical composition is administered to the individual no more than about 24 hours after the injury. In some embodiments, the pharmaceutical composition is administered to the tissue of the individual. In some embodiments, the pharmaceutical composition is administered to the individual systemically, e.g., orally. In some embodiments, the individual is an aged individual (e.g., a human individual of at least about 50 years old). In some embodiments, the pharmaceutical composition comprises rosiglitazone and PGI2. In some embodiments, the pharmaceutical composition comprises rosiglitazone and treprostinil.

In some embodiments, there is provided a method of treating injury to a muscle tissue in an individual, comprising administering an effective amount of a pharmaceutical composition comprising one or more FAO activators to the individual. In some embodiments, the pharmaceutical composition is administered to the individual no more than about 72 hours, no more than about 48 hours, or no more than about 24 hours after the muscle injury. In some embodiments, the pharmaceutical composition is administered to a muscle tissue of the individual, e.g., intramuscularly or subcutaneously. In some embodiments, the pharmaceutical composition is administered to the individual systemically, e.g., orally. In some embodiments, the individual is an aged individual (e.g., a human individual of at least about 50 years old). In some embodiments, the one or more FAO activators comprises an activator of a gene in the FAO pathway or lipid metabolism pathway. In some embodiments, the one or more FAO activators comprises one or more activators of PPARγ. In some embodiments, the one or more activators of PPARγ comprises a PPARγ agonist, such as rosiglitazone. In some embodiments, the one or more activators of PPARγ comprises a prostaglandin selected from the group consisting of PGI2, PGD2, and analogues thereof (e.g., treprostinil). In some embodiments, the one or more activators of PPARγ comprises rosiglitazone and PGI2, or rosiglitazone and treprostinil.

In some embodiments, there is provided a method of treating aging or a disease or condition associated with aging in an individual, comprising administering an effective amount of a pharmaceutical composition comprising an FAO activator to the individual. In some embodiments, the pharmaceutical composition is administered to a tissue of the individual. In some embodiments, the tissue is selected from the group consisting of a muscle tissue, a liver tissue, a heart tissue, a skin tissue and a hair follicle. In some embodiments, the pharmaceutical composition is administered to the individual systemically, e.g., orally. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the pharmaceutical composition comprises two or more FAO activators. In some embodiments, the individual is a human individual of at least about 50 years old.

In some embodiments, there is provided a method of treating aging or a disease or condition associated with aging in an individual, comprising administering an effective amount of a pharmaceutical composition comprising a PPARγ agonist to the individual. In some embodiments, the pharmaceutical composition is administered to a tissue of the individual. In some embodiments, the tissue is selected from the group consisting of a muscle tissue, a liver tissue, a heart tissue, a skin tissue and a hair follicle. In some embodiments, the pharmaceutical composition is administered to the individual systemically, e.g., orally. In some embodiments, the PPARγ agonist is rosiglitazone, or a salt, solvate, tautomer, or stereoisomer thereof. In some embodiments, the individual is a human individual of at least about 50 years old.

In some embodiments, there is provided a method of treating aging or a disease or condition associated with aging in an individual, comprising administering an effective amount of a pharmaceutical composition comprising a prostaglandin to the individual, wherein the prostaglandin is selected from the group consisting of PGI2, PGD2 and analogues thereof (e.g., treprostinil). In some embodiments, the pharmaceutical composition is administered to a tissue of the individual. In some embodiments, the tissue is selected from the group consisting of a muscle tissue, a liver tissue, a heart tissue, a skin tissue and a hair follicle. In some embodiments, the pharmaceutical composition is administered to the individual systemically, e.g., orally. In some embodiments, the individual is a human individual of at least about 50 years old.

In some embodiments, there is provided a method of treating aging or a disease or condition associated with aging in an individual, comprising administering an effective amount of a pharmaceutical composition comprising a PPARγ agonist (e.g., rosiglitazone) and a prostaglandin to the individual, wherein the prostaglandin is selected from the group consisting of PGI2, PGD2 and analogues thereof (e.g., treprostinil). In some embodiments, the pharmaceutical composition is administered to a tissue of the individual. In some embodiments, the tissue is selected from the group consisting of a muscle tissue, a liver tissue, a heart tissue, a skin tissue and a hair follicle. In some embodiments, the pharmaceutical composition is administered to the individual systemically, e.g., orally. In some embodiments, the individual is a human individual of at least about 50 years old. In some embodiments, the pharmaceutical composition comprises rosiglitazone and PGI2. In some embodiments, the pharmaceutical composition comprises rosiglitazone and treprostinil.

Also within the scope of the present application is the method of using fatty acid oxidation activation to mimic the benefits of exercise and nutrition to influence tissue (e.g., muscle) regeneration and degeneration in vivo.

In some embodiments, there is provided a method of providing one or more benefits of exercise and/or nutrition to a tissue (e.g., muscle tissue) of an individual, comprising administering an effective amount of a pharmaceutical composition comprising an FAO activator to the individual. In some embodiments, the tissue (e.g., muscle tissue) is injured. In some embodiments, the tissue (e.g., muscle tissue) is degenerated. In some embodiments, the pharmaceutical composition is administered to the individual once every 24 hours, every 48 hours, or every 72 hours. In some embodiments, the pharmaceutical composition is administered to the tissue (e.g., muscle tissue) of the individual, e.g., intramuscularly or subcutaneously. In some embodiments, the pharmaceutical composition is administered to the individual systemically, e.g., orally. In some embodiments, the FAO activator is an activator of a gene in the FAO pathway or lipid metabolism pathway. In some embodiments, the FAO activator is an activator of a gene selected from the group consisting of PPARα, PPARS, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the pharmaceutical composition comprises two or more FAO activators. In some embodiments, the individual is an aged individual (e.g., a human individual of at least about 50 years old).

In some embodiments, there is provided a method of providing one or more benefits of exercise and/or nutrition to a tissue (e.g., muscle tissue) of an individual, comprising administering an effective amount of a pharmaceutical composition comprising a PPARγ agonist to the individual. In some embodiments, the tissue (e.g., muscle tissue) is injured. In some embodiments, the tissue (e.g., muscle tissue) is degenerated. In some embodiments, the pharmaceutical composition is administered to the individual once every 24 hours, every 48 hours, or every 72 hours. In some embodiments, the pharmaceutical composition is administered to the tissue (e.g., muscle tissue) of the individual, e.g., intramuscularly or subcutaneously. In some embodiments, the pharmaceutical composition is administered to the individual systemically, e.g., orally. In some embodiments, the PPARγ agonist is rosiglitazone, or a salt, solvate, tautomer, or stereoisomer thereof. In some embodiments, the individual is an aged individual (e.g., a human individual of at least about 50 years old).

In some embodiments, there is provided a method of providing one or more benefits of exercise and/or nutrition to a tissue (e.g., muscle tissue) of an individual, comprising administering an effective amount of a pharmaceutical composition comprising a prostaglandin to the individual, wherein the prostaglandin is selected from the group consisting of PGI2, PGD2 and analogues thereof (e.g., treprostinil). In some embodiments, the tissue (e.g., muscle tissue) is injured. In some embodiments, the tissue (e.g., muscle tissue) is degenerated. In some embodiments, the pharmaceutical composition is administered to the individual once every 24 hours, every 48 hours, or every 72 hours. In some embodiments, the pharmaceutical composition is administered to the tissue (e.g., muscle tissue) of the individual, e.g., intramuscularly or subcutaneously. In some embodiments, the pharmaceutical composition is administered to the individual systemically, e.g., orally. In some embodiments, the individual is an aged individual (e.g., a human individual of at least about 50 years old).

In some embodiments, there is provided a method of providing one or more benefits of exercise and/or nutrition to a tissue (e.g., muscle tissue) of an individual, comprising administering an effective amount of a pharmaceutical composition comprising a PPARγ agonist (e.g., rosiglitazone) and a prostaglandin to the individual, wherein the prostaglandin is selected from the group consisting of PGI2, PGD2 and analogues thereof (e.g., treprostinil). In some embodiments, the tissue (e.g., muscle tissue) is injured. In some embodiments, the tissue (e.g., muscle tissue) is degenerated. In some embodiments, the pharmaceutical composition is administered to the individual once every 24 hours, every 48 hours, or every 72 hours. In some embodiments, the pharmaceutical composition is administered to the tissue (e.g., muscle tissue) of the individual, e.g., intramuscularly or subcutaneously. In some embodiments, the pharmaceutical composition is administered to the individual systemically, e.g., orally. In some embodiments, the individual is an aged individual (e.g., a human individual of at least about 50 years old). In some embodiments, the pharmaceutical composition comprises rosiglitazone and PGI2. In some embodiments, the pharmaceutical composition comprises rosiglitazone and treprostinil.

In some embodiments, the one or more benefits of exercise and/or nutrition comprises increase in myogenesis, increase in muscle regeneration, decrease in muscle degeneration, increase in tissue regeneration, decrease in tissue degeneration, increase in muscle volume, increase in muscle mass, increase in muscle glucose and fat metabolism, increase in muscle insulin sensitivity, increase in muscle stamina, and/or increase in muscle strength.

Also provided are compositions (such as pharmaceutical compositions) comprising any one or more of the FAO activators including activators of PPARγ, such as PPARγ agonists, and/or PGI2, PGD2 or analogues thereof described herein for use in any one of the methods described herein.

Generally, dosages, schedules, and routes of administration of the pharmaceutical compositions comprising the one or more FAO activators may be determined according to the size and condition of the individual, and according to standard pharmaceutical practice. Exemplary routes of administration include oral, rectal, nasal, topical (including buccal and sublingual), transdermal, vaginal or parenteral (including intramuscular, subcutaneous and intravenous. In some embodiments, the pharmaceutical composition is administered locally to a muscle tissue in the individual. In some embodiments, the pharmaceutical composition is administered subcutaneously. In some embodiments, the pharmaceutical composition is administered intramuscularly. In some embodiments, the pharmaceutical composition is administered by injection. In some embodiments, the pharmaceutical composition is administered to the individual systemically. In some embodiments, the pharmaceutical composition is administered to the individual orally.

The dose of the one or more FAO activators administered to an individual may vary according to, for example, the particular type of FAO activator(s) being administered, the route of administration, and the particular type of muscle disease or conditions being treated. The amount should be sufficient to produce a desirable response, such as a therapeutic response against the disease or condition, but without severe toxicity or adverse events. In some embodiments, the one or more FAO activators is administered at a therapeutically effective amount.

In some embodiments, the pharmaceutical composition is administered to the individual once. In some embodiments, the pharmaceutical composition is administered to the individual more than once, such as any one of 2, 3, 4, 5, 6, or more times. In some embodiments, the pharmaceutical composition may conveniently be presented in a once daily or as divided dose administered at appropriate intervals, for example as one does per 24, 48 or 72 hours. In some embodiments, the pharmaceutical composition is administered once every 24 hours, once every 36 hours, once every 48 hours, once every 60 hours, or once every 72 hours, including any value or range in between these values.

In some embodiments, the pharmaceutical composition is administered to the individual within about 72 hours from the muscle injury, such as within about any one of 60 hours, 48 hours, 36 hours, 24 hours, 12 hours, 6 hours, or less, including any value or range in between these values, from the muscle injury. In some embodiments, administration of rosiglitazone to an individual at the 24 and 48 hour time points after injury to a muscle tissue improved skeletal muscle regeneration in vivo.

### Administration of Tissuegenic Cells

In some embodiments, there is provided a method of treating a disease or condition associated with a tissue (e.g., a muscle disease or condition) in an individual, comprising administering an effective amount of a pharmaceutical composition comprising tissuegenic cells (such as myogenic cells, e.g., myoblasts and/or myocytes) to the tissue (e.g., muscle tissue) of the individual, wherein the tissuegenic cells are contacted with an FAO activator prior to the administration of the pharmaceutical composition. In some embodiments, the disease or condition is tissue injury (e.g., muscle injury). In some embodiments, the disease or condition is tissue degeneration (e.g., muscle degeneration). In some embodiments, the disease or condition is tissue fibrosis (e.g., muscle fibrosis). In some embodiments, the disease or condition is aging. In some embodiments, the tissue is selected from the group consisting of a muscle tissue, a liver tissue, a heart tissue, a skin tissue and a hair follicle. In some embodiments, the tissuegenic cells (e.g., myogenic cells) are contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the tissuegenic cells (e.g., myogenic cells) are contacted with two or more FAO activators. In some embodiments, the individual is an aged individual (e.g., a human individual of at least about 50 years old). In some embodiments, the method further comprises contacting the tissuegenic cells with the FAO activator prior to the administration of the pharmaceutical composition.

In some embodiments, there is provided a method of treating a disease or condition associated with a tissue (e.g., a muscle disease or condition) in an individual, comprising administering an effective amount of a pharmaceutical composition comprising tissuegenic cells (such as myogenic cells, e.g., myoblasts and/or myocytes) to the tissue (e.g., muscle tissue) of the individual, wherein the tissuegenic cells are contacted with a PPARγ agonist prior to the administration of the pharmaceutical composition. In some embodiments, the disease or condition is tissue injury (e.g., muscle injury). In some embodiments, the disease or condition is tissue degeneration (e.g., muscle degeneration). In some embodiments, the disease or condition is tissue fibrosis (e.g., muscle fibrosis). In some embodiments, the disease or condition is aging. In some embodiments, the tissue is selected from the group consisting of a muscle tissue, a liver tissue, a heart tissue, a skin tissue and a hair follicle. In some embodiments, the tissuegenic cells (e.g., myogenic cells) are contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the individual is an aged individual (e.g., a human individual of at least about 50 years old). In some embodiments, the method further comprises contacting the tissuegenic cells with the PPARγ agonist prior to the administration of the pharmaceutical composition.

In some embodiments, there is provided a method of treating a disease or condition associated with a tissue (e.g., a muscle disease or condition) in an individual, comprising administering an effective amount of a pharmaceutical composition comprising tissuegenic cells (such as myogenic cells, e.g., myoblasts and/or myocytes) to the tissue (e.g., muscle tissue) of the individual, wherein the tissuegenic cells are contacted with a prostaglandin prior to the administration of the pharmaceutical composition, and wherein the prostaglandin is selected from the group consisting of PGI2, PGD2 and analogues thereof (e.g., treprostinil). In some embodiments, the disease or condition is tissue injury (e.g., muscle injury). In some embodiments, the disease or condition is tissue degeneration (e.g., muscle degeneration). In some embodiments, the disease or condition is tissue fibrosis (e.g., muscle fibrosis). In some embodiments, the disease or condition is aging. In some embodiments, the tissue is selected from the group consisting of a muscle tissue, a liver tissue, a heart tissue, a skin tissue and a hair follicle. In some embodiments, the tissuegenic cells (e.g., myogenic cells) are contacted with the prostaglandin for no more than about 72 hours, no more than about 48 hours, or no more than about 24 hours. In some embodiments, the individual is an aged individual (e.g., a human individual of at least about 50 years old). In some embodiments, the method further comprises contacting the tissuegenic cells with the prostaglandin prior to the administration of the pharmaceutical composition.

In some embodiments, there is provided a method of treating a disease or condition associated with a tissue (e.g., a muscle disease or condition) in an individual, comprising administering an effective amount of a pharmaceutical composition comprising tissuegenic cells (such as myogenic cells, e.g., myoblasts and/or myocytes) to the tissue (e.g., muscle tissue) of the individual, wherein the tissuegenic cells are contacted with a PPARγ agonist (e.g., rosiglitazone) and a prostaglandin prior to the administration of the pharmaceutical composition, and wherein the prostaglandin is selected from the group consisting of PGI2, PGD2 and analogues thereof (e.g., treprostinil). In some embodiments, the disease or condition is tissue injury (e.g., muscle injury). In some embodiments, the disease or condition is tissue degeneration (e.g., muscle degeneration). In some embodiments, the disease or condition is tissue fibrosis (e.g., muscle fibrosis). In some embodiments, the disease or condition is aging. In some embodiments, the tissue is selected from the group consisting of a muscle tissue, a liver tissue, a heart tissue, a skin tissue and a hair follicle. In some embodiments, the tissuegenic cells (e.g., myogenic cells) are contacted with the PPARγ agonist and the prostaglandin for no more than about 72 hours, no more than about 48 hours, or no more than about 24 hours. In some embodiments, the individual is an aged individual (e.g., a human individual of at least about 50 years old). In some embodiments, the PPARγ agonist is rosiglitazone and the prostaglandin is PGI2. In some embodiments, the PPARγ agonist is rosiglitazone and the prostaglandin istreprostinil. In some embodiments, the method further comprises contacting the tissuegenic cells with the PPARγ agonist and the prostaglandin prior to the administration of the pharmaceutical composition.

In some embodiments, there is provided a method of treating a muscle disease or condition in an individual, comprising: (1) contacting myogenic cells (e.g., myoblasts or myocytes) with an FAO activator to provide a pharmaceutical composition comprising the myogenic cells or differentiated cells thereof; and (2) administering an effective amount of the pharmaceutical composition to a muscle tissue of the individual. In some embodiments, the muscle disease or condition is muscle injury. In some embodiments, the muscle disease or condition is muscle degeneration. In some embodiments, the method comprises contacting the myogenic cells with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the myogenic cells with two or more FAO activators. In some embodiments, the individual is an aged individual (e.g., a human individual of at least about 50 years old).

In some embodiments, there is provided a method of treating a muscle disease or condition in an individual, comprising: (1) contacting myogenic cells (e.g., myoblasts or myocytes) with a PPARγ agonist to provide a pharmaceutical composition comprising the myogenic cells or differentiated cells thereof; and (2) administering an effective amount of the pharmaceutical composition to a muscle tissue of the individual. In some embodiments, the muscle disease or condition is muscle injury. In some embodiments, the muscle disease or condition is muscle degeneration. In some embodiments, the method comprises contacting the myogenic cells with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the individual is an aged individual (e.g., a human individual of at least about 50 years old).

In some embodiments, there is provided a method of treating a muscle disease or condition in an individual, comprising: (1) contacting myogenic cells (e.g., myoblasts or myocytes) with a prostaglandin to provide a pharmaceutical composition comprising the myogenic cells or differentiated cells thereof, wherein the prostaglandin is selected from the group consisting of PGI2, PGD2 and analogues thereof (e.g., treprostinil); and (2) administering an effective amount of the pharmaceutical composition to a muscle tissue of the individual. In some embodiments, the muscle disease or condition is muscle injury. In some embodiments, the muscle disease or condition is muscle degeneration. In some embodiments, the method comprises contacting the myogenic cells with the prostaglandin for no more than about 72 hours, no more than about 48 hours, or no more than about 24 hours. In some embodiments, the individual is an aged individual (e.g., a human individual of at least about 50 years old).

In some embodiments, there is provided a method of treating a muscle disease or condition in an individual, comprising: (1) contacting myogenic cells (e.g., myoblasts or myocytes) with a PPARγ agonist (e.g., rosiglitazone) and a prostaglandin to provide a pharmaceutical composition comprising the myogenic cells or differentiated cells thereof, wherein the prostaglandin is selected from the group consisting of PGI2, PGD2 and analogues thereof (e.g., treprostinil); and (2) administering an effective amount of the pharmaceutical composition to a muscle tissue of the individual. In some embodiments, the muscle disease or condition is muscle injury. In some embodiments, the muscle disease or condition is muscle degeneration. In some embodiments, the method comprises contacting the myogenic cells with the PPARγ agonist and the prostaglandin for no more than about 72 hours, no more than about 48 hours, or no more than about 24 hours. In some embodiments, the individual is an aged individual (e.g., a human individual of at least about 50 years old). In some embodiments, the PPARγ agonist is rosiglitazone and the prostaglandin is PGI2. In some embodiments, the PPARγ agonist is rosiglitazone and the prostaglandin istreprostinil.

In some embodiments, there is provided a method of treating injury to a muscle tissue in an individual, comprising administering an effective amount of a pharmaceutical composition comprising myogenic cells to the muscle tissue of the individual, wherein the myogenic cells are contacted with an FAO activator prior to the administration of the pharmaceutical composition. In some embodiments, the pharmaceutical composition is administered to the individual no more than about 72 hours, no more than about 48 hours, or no more than about 24 hours after the muscle injury. In some embodiments, the myogenic cells are contacted with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the myogenic cells are contacted with two or more FAO activators. In some embodiments, the individual is an aged individual (e.g., a human individual of at least about 50 years old).

In some embodiments, there is provided a method of treating injury to a muscle tissue in an individual, comprising administering an effective amount of a pharmaceutical composition comprising myogenic cells to the muscle tissue of the individual, wherein the myogenic cells are contacted with a PPARγ agonist prior to the administration of the pharmaceutical composition. In some embodiments, the pharmaceutical composition is administered to the individual no more than about 72 hours, no more than about 48 hours, or no more than about 24 hours after the muscle injury. In some embodiments, the myogenic cells are contacted with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the individual is an aged individual (e.g., a human individual of at least about 50 years old).

In some embodiments, there is provided a method of treating injury to a muscle tissue in an individual, comprising administering an effective amount of a pharmaceutical composition comprising myogenic cells (e.g., myoblasts or myocytes) to a muscle tissue of the individual, wherein the myogenic cells are contacted with a prostaglandin prior to the administration of the pharmaceutical composition, wherein the prostaglandin is selected from the group consisting of PGI2, PGD2 and analogues thereof (e.g., treprostinil). In some embodiments, the muscle disease or condition is muscle injury. In some embodiments, the muscle disease or condition is muscle degeneration. In some embodiments, the myogenic cells are contacted with the PGI2 or analogue thereof for no more than about 72 hours, no more than about 48 hours, or no more than about 24 hours. In some embodiments, the individual is an aged individual (e.g., a human individual of at least about 50 years old).

In some embodiments, there is provided a method of treating injury to a muscle tissue in an individual, comprising administering an effective amount of a pharmaceutical composition comprising myogenic cells (e.g., myoblasts or myocytes) to a muscle tissue of the individual, wherein the myogenic cells are contacted with a PPARγ agonist (e.g., rosiglitazone) and a prostaglandin prior to the administration of the pharmaceutical composition, wherein the prostaglandin is selected from the group consisting of PGI2, PGD2 and analogues thereof (e.g., treprostinil). In some embodiments, the muscle disease or condition is muscle injury. In some embodiments, the muscle disease or condition is muscle degeneration. In some embodiments, the myogenic cells are contacted with the PPARγ agonist and the prostaglandin for no more than about 72 hours, no more than about 48 hours, or no more than about 24 hours. In some embodiments, the individual is an aged individual (e.g., a human individual of at least about 50 years old). In some embodiments, the PPARγ agonist is rosiglitazone and the prostaglandin is PGI2. In some embodiments, the PPARγ agonist is rosiglitazone and the prostaglandin istreprostinil.

In some embodiments, there is provided a method of treating injury to a muscle tissue in an individual, comprising: (1) contacting myogenic cells with an FAO activator to provide a pharmaceutical composition comprising the myogenic cells or differentiated cells thereof; and (2) administering an effective amount of the pharmaceutical composition to the muscle tissue of the individual. In some embodiments, the pharmaceutical composition is administered to the individual no more than about 72 hours, no more than about 48 hours, or no more than about 24 hours after the muscle injury. In some embodiments, the method comprises contacting the myogenic cells with the FAO activator for no more than about 72 hours, no more than about 48 hours, or no more than about 24 hours. In some embodiments, the FAO activator is an activator of a gene in the FAO pathway or lipid metabolism pathway. In some embodiments, the FAO activator is an activator of a gene selected from the group consisting of PPARα, PPARS, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the method comprises contacting the myogenic cells with two or more FAO activators. In some embodiments, the individual is an aged individual (e.g., a human individual of at least about 50 years old).

In some embodiments, there is provided a method of treating injury to a muscle tissue in an individual, comprising: (1) contacting myogenic cells with a PPARγ agonist to provide a pharmaceutical composition comprising the myogenic cells or differentiated cells thereof; and (2) administering an effective amount of the pharmaceutical composition to the muscle tissue of the individual. In some embodiments, the pharmaceutical composition is administered to the individual no more than about 72 hours, no more than about 48 hours, or no more than about 24 hours after the muscle injury. In some embodiments, the method comprises contacting the myogenic cells with the PPARγ agonist for no more than about 72 hours, no more than about 48 hours, or no more than about 24 hours. In some embodiments, the PPARγ agonist is rosiglitazone, or a salt, solvate, tautomer, or stereoisomer thereof. In some embodiments, the individual is an aged individual (e.g., a human individual of at least about 50 years old).

In some embodiments, there is provided a method of treating injury to a muscle tissue in an individual, comprising: (1) contacting myogenic cells (e.g., myoblasts or myocytes) with a prostaglandin to provide a pharmaceutical composition comprising the myogenic cells or differentiated cells thereof, wherein the prostaglandin is selected from the group consisting of PGI2, PGD2 and analogues thereof (e.g., treprostinil); and (2) administering an effective amount of the pharmaceutical composition to a muscle tissue of the individual. In some embodiments, the muscle disease or condition is muscle injury. In some embodiments, the muscle disease or condition is muscle degeneration. In some embodiments, the method comprises contacting the myogenic cells with the PGI2 or analogue thereof for no more than about 72 hours, no more than about 48 hours, or no more than about 24 hours. In some embodiments, the individual is an aged individual (e.g., a human individual of at least about 50 years old).

In some embodiments, there is provided a method of treating injury to a muscle tissue in an individual, comprising: (1) contacting myogenic cells (e.g., myoblasts or myocytes) with a PPARγ agonist (e.g., rosiglitazone) and a prostaglandin to provide a pharmaceutical composition comprising the myogenic cells or differentiated cells thereof, wherein the prostaglandin is selected from the group consisting of PGI2, PGD2 and analogues thereof (e.g., treprostinil); and (2) administering an effective amount of the pharmaceutical composition to a muscle tissue of the individual. In some embodiments, the muscle disease or condition is muscle injury. In some embodiments, the muscle disease or condition is muscle degeneration. In some embodiments, the method comprises contacting the myogenic cells with the PPARγ agonist and the prostaglandin for no more than about 72 hours, no more than about 48 hours, or no more than about 24 hours. In some embodiments, the individual is an aged individual (e.g., a human individual of at least about 50 years old). In some embodiments, the PPARγ agonist is rosiglitazone and the prostaglandin is PGI2. In some embodiments, the PPARγ agonist is rosiglitazone and the prostaglandin istreprostinil.

Suitable tissuegenic cells include, but are not limited to stem cells, progenitor cells, ESC and iPSC, reprogrammed cells, transdifferentiated cells, or differentiated cells produced from such stem cells, precursor cells, or combinations thereof. Suitable myogenic cells include, but are not limited to muscle stem cells (e.g., satellite cells), embryonic and fetal myoblasts, myoblasts produced from ESC or iPSC, reprogrammed myogenic cells (e.g., rejuvenated and/or de-differentiated myogenic cells) or transdifferentiated myogenic cells, or differentiated cells produced from such muscle stem cells, myoblasts, or reprogrammed myogenic cells.

The tissuegenic cells (e.g., myogenic cells) may be obtained from various sources. In some embodiments, the tissuegenic cells (e.g., myogenic cells) are autologous. In some embodiments, the tissuegenic cells (e.g., myogenic cells) are allogenic. In some embodiments, the tissuegenic cells (e.g., myogenic cells) are non-immunogenic to the individual. In some embodiments, the tissuegenic cells (e.g., myogenic cells) are produced from a cell line. In some embodiments, the tissuegenic cells (e.g., myogenic cells) are not produced from an immortal cell line. In some embodiments, the tissuegenic cells (e.g., myogenic cells) are produced from primary cells obtained from the individual. In some embodiments, the tissuegenic cells (e.g., myogenic cells) are produced from primary cells obtained from a donor.

Muscle stem cells may be obtained using known methods in the art. See, for example, by culturing isolated muscle stem cells from young individuals and culturing the muscle stem cells by differential adhesion (e.g., Skuk, 2010), by FACS sorting of adult muscle stem cells (e.g., Conboy, 2010), and by preparing muscle stem cells from ESC or iPSC (e.g., Darabi, 2008; Borchin, 2013; Shelton, 2016), which are incorporated herein by reference in their entirety.

Myoblasts may be produced from ESC or iPSC using known methods in the art. See, for example, Darabi, 2008; Borchin, 2013; Shelton, 2016, which are incorporated herein by reference in their entirety. Reprogrammed (e.g., rejuvenated and/or de-differentiated) myoblasts may be produced using methods described in PCT/CN2019/088977 and PCT/CN2020/092615. Additionally, muscle progenitor cells such as muscle stem cells and myoblasts may be produced by direct reprogramming of adult somatic cells using myogenic transcription factor(s) and/or small molecule drugs, for example, transdifferentiation of mouse fibroblasts by transient expression of MyoD in combination with GSK3P inhibitor (e.g., CHIR99021), TGF-β inhibitor (e.g., RepSox), and/or cAMP agonist (e.g., Forskolin). See, Bar-Nur, 2018, the contents of which are incorporated herein by reference in its entirety. Myoblasts may be proliferated without differentiation by culturing myoblasts under suitable conditions, for example in a proliferation medium comprising DMEM and about 20% FBS, and passaged before about 80% confluency each time.

Myocytes may be produced from myoblasts by culturing the myoblasts under suitable conditions. For example, myoblasts may be allowed to reach 100% confluency and cultured in a differentiation medium comprising a DMEM/F12 or DMEM medium, supplemented with about 2% KnockOut Serum Replacement or about 2% horse serum, and about 1% L-glutamine for about 2 days.

In some embodiments, the method further comprises administering to the individual an effective amount of an immunosuppressant to minimize rejection of the tissuegenic cells (e.g., myogenic cells). Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF alpha, blockers, a biological agent that targets an inflammatory cytokine, and Non-Steroidal Anti-Inflammatory Drug (NSAIDs).

Any of the methods described herein may further comprise one or more steps for in vitro production of the tissuegenic cells (e.g., myogenic cells). Any suitable methods for in vitro proliferation and/or differentiation of tissuegenic cells (e.g., myogenic cells) may be used. See, for example, Chua et al., 2019; and Fukawa et al., 2016. In some embodiments, the method comprises obtaining the tissuegenic cells (such as myogenic cells, e.g., muscle stem cells, myoblasts, and/or myocytes) from the individual or a donor. In some embodiments, the method comprises any one of the methods of producing reprogrammed myogenic cells from adult myogenic cells or adult somatic cells (e.g., fibroblasts). In some embodiments, the method comprises culturing the tissuegenic cells (e.g., myogenic cells) in vitro under conditions that allow proliferation of the tissuegenic cells. In some embodiments, the method comprises culturing the myoblasts in vitro under conditions that allow proliferation of the myogenic cells (e.g., myoblasts and/or myocytes) without differentiation. In some embodiments, the myoblasts are cultured in a proliferation medium comprising DMEM/F12 with about 20% FBS and about 1% L-glutamine. In some embodiments, the method comprises culturing tissuegenic cells (such as myogenic cells, e.g., muscle stem cells, myoblasts, and/or myocytes) in vitro under conditions that allow differentiation of the tissuegenic cells. In some embodiments, the myoblasts are cultured in a differentiation medium comprising DMEM/F12 or DMEM, with about 2% KnockOut Serum Replacement or about 2% horse serum, and 1% L-glutamine. In some embodiments, the method comprises culturing tissuegenic cells (e.g., myogenic cells) in a differentiation medium in the presence of the one or more FAO activators such as PPARγ agonist and/or PGI2 or analogue thereof. In some embodiments, the tissuegenic cells (e.g., myogenic cells) are cultured in vitro for no more than about any one of 72 hours, 60 hours, 48 hours, 36 hours, 24 hours, 12 hours, or 6 hours prior to administration to the individual. In some embodiments, the tissuegenic cells (e.g., myogenic cells) are seeded at high density (e.g., at least about 80% confluency). In some embodiments, the tissuegenic cells (e.g., myogenic cells) are seeded at low density (e.g., lower than about 80% confluency).

The pharmaceutical composition comprising myogenic cells described herein may comprise tissuegenic cells (e.g., myogenic cells), their progeny, and cells that differentiated from the tissuegenic cells (e.g., myogenic cells). The pharmaceutical composition may be a suspension of cells, or a tissue construct (e.g., a muscle construct). In some embodiments, the pharmaceutical composition is a solution suitable for injection. In some embodiments, the pharmaceutical composition is a hydrogel suitable for surgical implantation. In some embodiments, the pharmaceutical composition comprises a pharmaceutically acceptable carrier. In some embodiments, the pharmaceutically composition comprises a cell adhesion molecule, such as fibrin. In some embodiments, the tissuegenic cells (e.g., myogenic cells) are intermixed with the carrier. In some embodiments, the pharmaceutically composition comprises extracellular matrix molecules. In some embodiments, the pharmaceutically composition comprises MATRIGEL®.

In some embodiments, the method comprises implanting a tissue construct in the individual. In some embodiments, the method comprises implanting a muscle construct at the muscle tissue of the individual. Any of the methods described herein may further comprise one or more steps for preparing a muscle construct. Any suitable methods for preparing muscle constructs may be used. See, for example, Velcro-anchored fibrin constructs (e.g., Hinds et al., 2011), and suture-anchored fibrin constructs (e.g., Khodabukus and Baar, 2009), three-dimensional bio-printing of muscle constructs such as by coaxial printing (e.g., Testa, 2018) and using a tissue-derived bio-ink (e.g., Choi, 2019), and culturing muscle progenitor cells on three-dimensional printed molds (e.g., Capel, 2019). In some embodiments, the method comprises culturing myogeniccells (e.g., myoblasts) in a hydrogel carrier, such as a carrier comprising MATRIGEL® and fibrin, to produce a muscle construct. In some embodiments, the myogeniccells (e.g., myoblasts) are cultured on the surface of a hydrogel, such as fibrin, anchored with sutures to produce a muscle construct. In some embodiments, the myogeniccells are cultured within a three-dimensional (“3D”) solid mold to produce a pre-shaped muscle construct. In some embodiments, the myogeniccells are 3D-printed with ink to produce a defined 3D muscle construct.

The present application also provides compositions (such as pharmaceutical compositions) comprising tissuegenic cells (such as myogenic cells, e.g., myoblasts and/or myocytes) or differentiated cells thereof that can be used in any one of the methods of treatment described herein. Also provided are tissue constructs (e.g., muscle constructs) comprising tissuegenic cells (such as myogenic cells, e.g., myoblasts and/or myocytes) or differentiated cells thereof.

Generally, dosages, schedules, and routes of administration of the pharmaceutical compositions comprising the tissuegenic cells (e.g., myogenic cells) may be determined according to the size and condition of the individual, and according to standard pharmaceutical practice. Exemplary routes of administration include intravenous, intra-arterial, intraperitoneal, intramuscular, subcutaneous, or transdermal. In some embodiments, the pharmaceutical composition is administered subcutaneously. In some embodiments, the pharmaceutical composition is administered intramuscularly. In some embodiments, the pharmaceutical composition is administered by injection. In some embodiments, the pharmaceutical composition is administered by surgical implantation.

The dose of the cells administered to an individual may vary according to, for example, the particular type of cells being administered, the route of administration, and the particular type of diseases or conditions (e.g., muscle diseases or conditions) being treated. The amount should be sufficient to produce a desirable response, such as a therapeutic response against the disease or condition, but without severe toxicity or adverse events. In some embodiments, the myogenic cells or differentiated cells thereof are administered at a therapeutically effective amount. In some embodiments, the pharmaceutical composition comprises at least about any one of 10³, 10⁴, 10⁵, 10⁶, 10⁷ or more cells, including any value or range in between these values.

In some embodiments, the pharmaceutical composition is administered to the individual once. In some embodiments, the pharmaceutical composition is administered to the individual more than once, such as any one of 2, 3, 4, 5, 6, or more times. In some embodiments, the pharmaceutical composition is administered once every 24 hours, once every 36 hours, once every 48 hours, once every 60 hours, or once every 72 hours, including any value or range in between these values. In some embodiments, the pharmaceutical composition is administered to the individual within about 72 hours from the tissue injury (e.g., muscle injury), such as within about any one of 60 hours, 48 hours, 36 hours, 24 hours, 12 hours, 6 hours, or less, including any value or range in between these values, from the tissue injury (e.g., muscle injury).

### Pharmaceutical Compositions

The present application provides compositions such as pharmaceutical compositions useful for any one of the methods of treatment described herein.

The pharmaceutical compositions may comprise one or more pharmaceutically acceptable carrier. As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration. Techniques for formulation and administration of drugs may be found in “Remington's Pharmaceutical Sciences,” Mack Publishing Co., Easton, Pa., latest edition, which is incorporated herein by reference.

The pharmaceutical compositions described herein may include other agents, excipients, or stabilizers to improve properties of the composition. Examples of pharmaceutically acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the embodiments are sterile compositions. Pharmaceutical compositions may be prepared using compounding techniques known or that become available to those skilled in the art. The final form may be sterile and may also be able to pass readily through an injection device such as a hollow needle. The proper viscosity may be achieved and maintained by the proper choice of solvents or excipients. In some embodiments, the composition is suitable for administration to a human.

The pharmaceutical compositions and compounds described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

In some embodiments, there is provided a pharmaceutical composition comprising one or more FAO activators and a pharmaceutically acceptable salt thereof. In some embodiments, the one or more FAO activators is a PPARγ agonist. In some embodiments, the PPARγ agonist is rosiglitazone, or a salt, solvate, tautomer, or stereoisomer thereof. In some embodiments, the one or more FAO activators is a prostaglandin selected from the group consisting of PGI2, PGD2 and analogues thereof (e.g., treprostinil) and salts, solvates, tautomers, and stereoisomers thereof. In some embodiments, the one or more FAO activators is PGI2, or a salt, solvate, tautomer, or stereoisomer thereof. In some embodiments, the one or more FAO activators is treprostinil, or a salt, solvate, tautomer, or stereoisomer thereof. In some embodiments, the one or more FAO activators are rosiglitazone and PGI2. In some embodiments, the one or more FAO activators are rosiglitazone and treprostinil. The pharmaceutical composition may be formulated for oral, rectal, nasal, topical (including buccal and sublingual), transdermal, vaginal or parenteral (including intramuscular, subcutaneous and intravenous) administration in liquid or solid form or in a form suitable for administration by inhalation or insufflation. In some embodiments, the pharmaceutical composition is formulated for intramuscular or subcutaneous administration. In some embodiments, the pharmaceutical composition is formulated for oral administration.

For oral administration, the one or more FAO activators (e.g., PPARγ agonist and/or PGD2, PGI2 or analogue thereof) may be provided in a solid form, or as a solution, emulsion, or suspension. For example, the pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical compositions according to the present application may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

In some embodiments, there is provided a pharmaceutical composition comprising tissuegenic cells (such as myogenic cells, e.g., myoblasts and/or myocytes), wherein the tissuegenic cells (e.g., myogenic cells) are contacted with one or more FAO activators for no more than about 72 hours. In some embodiments, the tissuegenic cells (e.g., myogenic cells) are contacted with the one or more FAO activators for no more than about 48 hours. In some embodiments, the tissuegenic cells (e.g., myogenic cells) are contacted with the one or more FAO activators for no more than about 24 hours. In some embodiments, the one or more FAO activators comprises an activator of a gene in the FAO pathway or lipid metabolism pathway. In some embodiments, the one or more FAO activators comprises an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB. In some embodiments, the one or more FAO activators comprises a PPARγ agonist. In some embodiments, the PPARγ agonist is rosiglitazone, or a salt, solvate, tautomer, or stereoisomer thereof. In some embodiments, the one or more FAO activators comprises a prostaglandin selected from the group consisting of PGI2, PGD2 and analogues thereof (e.g., treprostinil) and salts, solvates, tautomers, and stereoisomers thereof. In some embodiments, the one or more FAO activators are rosiglitazone and PGI2. In some embodiments, the one or more FAO activators are rosiglitazone and treprostinil. In some embodiments, the pharmaceutical composition is formulated for intramuscular administration. In some embodiments, the pharmaceutical composition is formulated for subcutaneous administration. In some embodiments, the pharmaceutical composition is formulated for surgical implantation. In some embodiments, the pharmaceutical composition is formulated for injection.

In some embodiments, there is provided a composition that comprises of any one or more activators that increases the expression of FAO and lipid regulating genes such as but not limited to the nuclear hormone receptors PPARA, PPARD, PPARG, RXRB, RXRG, NCOA1, NCOA2; the upstream fatty acid transporters FABP3, FABP4, CD36, SCARB1, FATP1-6; a variety of lipases including LPL; the rate-limiting carnitine palmitoyl-transferases CPT1A and CPT1B; the carnitine acetylase CRAT; the acyl-CoA dehydrogenases ACADs and hydroxyacyl-CoA dehydrogenases HADHs; and the mitochondrial electron transfer flavoproteins ETFA and ETFB, which can promote myogenic differentiation.

In some embodiments, there is provided a composition that comprises of any one or more PPAR agonists, solvates, hydrates or pharmaceutically acceptable salts thereof, including any one or more of the PPAR agonists described in Section IV. In some embodiments, the composition comprises PGD2, PGI2, an analogue thereof (e.g., treprostinil), or a salt, solvate, tautomer, or stereoisomer thereof. In some embodiments, the composition activates fatty acid oxidation, and enhances tissue (e.g., muscle) regeneration. An exemplary PPAR agonist provided herein is a thiazolidinedione, solvates, hydrates or pharmaceutically acceptable salts thereof.

In some embodiments, there is provided a composition comprising rosiglitazone, solvates, hydrates or pharmaceutically acceptable salts thereof. In some embodiments, the composition further comprises PGI2, PGD2 or an analogue thereof (e.g., treprostinil).

### IV. Fatty acid oxidation activators

The methods and compositions described herein use one or more (e.g., 1, 2, 3, or more) FAO activators. In some embodiments, the one or more FAO activators has one or more of the following properties: (i) increases mitochondrial FAO in a tissuegenic cell (e.g., myogenic cell); (ii) increases mitochondrial oxygen consumption in a tissuegenic cell (e.g., myogenic cell); (iii) does not affect mitochondrial biogenesis in a tissuegenic cell (e.g., myogenic cell); (v) does not affect membrane potential of a tissuegenic cell (e.g., myogenic cell); (vi) increases expression and/or activity of MyoD (e.g., MyoD1) in a tissuegenic cell (e.g., myogenic cell); (vii) increases expression and/or activity of PPARγ in a tissuegenic cell (e.g., myogenic cell); (viii) transiently increases expression and/or activity of PPARα in a tissuegenic cell (e.g., myogenic cell); (ix) increases expression and/or activity of PAX7 in a tissuegenic cell (e.g., myogenic cell); (x) increases expression and/or activity of MyoG in a tissuegenic cell (e.g., myogenic cell)s; (xi) increases expression and/or activity of Myh3 in a tissuegenic cell (e.g., myogenic cell)s; (xii) increases level of H3K9acin a tissuegenic cell (e.g., myogenic cell); and (xiii) increases expression and/or activity of Ki67 in a tissuegenic cell (e.g., myogenic cell).

In some embodiments, the one or more FAO activators increases mitochondrial FAO in a tissuegenic cell (e.g., myogenic cell, such as myoblast or myocyte). In some embodiments, the one or more FAO activators increases mitochondrial FAO in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase mitochondrial FAO in the tissuegenic cell (e.g., myogenic cell) after about 72 hours, about 84 hours, about 96 hours or longer, including any value or range in between these values. The level of FAO may be determined using any known methods in the art, for example, by metabolomics and lipidomics analysis using mass spectrometry. In some embodiments, the level of mitochondrial FAO increases by at least any one of 10%, 20%, 50%, 2×, 3×, 5×, 10× or more, including any value or range in between these values.

In some embodiments, the one or more FAO activators increases mitochondrial oxygen consumption in a tissuegenic cell (e.g., myogenic cell, such as a myoblast or a myocyte). In some embodiments, the one or more FAO activators increases mitochondrial oxygen consumption in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase mitochondrial oxygen consumption in the tissuegenic cell (e.g., myogenic cell) after about 72 hours, about 84 hours, about 96 hours or longer, including any value or range in between these values. Mitochondrial oxygen consumption may be determined using any known methods in the art, for example, by Seahorse analysis. In some embodiments, the method increases maximal mitochondrial oxygen consumption. In some embodiments, the method increases basal mitochondrial oxygen consumption. In some embodiments, the method increases both maximal mitochondrial oxygen consumption and basal mitochondrial oxygen consumption. In some embodiments, the mitochondrial oxygen consumption increases by at least any one of 10%, 20%, 50%, 2×, 3×, 5×, 10× or more, including any value or range in between these values.

In some embodiments, the one or more FAO activators does not affect mitochondrial biogenesis in a tissuegenic cell (e.g., myogenic cell, such as myoblast or myocyte). Mitochondrial biogenesis may be determined using any known methods in the art, for example, by determining mitochondrial volume via immunostaining or staining by MitoTracker, or by determining mitochondrial DNA copy number via quantitative PCR. In some embodiments, the one or more FAO activators does not change mitochondrial biogenesis in the tissuegenic cell (e.g., myogenic cell) by more than 50%, 40%, 30%, 20%, 10% or less, including any value or range in between these values.

In some embodiments, the one or more FAO activators does not affect membrane potential of a tissuegenic cell (e.g., myogenic cell, such as myoblast or myocyte). Membrane potential may be determined using any known methods in the art, for example, by fluorescent staining using JC1 dyes. In some embodiments, the one or more FAO activators does not change membrane potential of the tissuegenic cell (e.g., myogenic cell) by more than 50%, 40%, 30%, 20%, 10% or less, including any value or range in between these values.

In some embodiments, the one or more FAO activators increases expression and/or activity of PAX7 in a tissuegenic cell (e.g., myogenic cell, e.g., myoblast or myocyte). In some embodiments, the one or more FAO activators increases the expression and/or activity of PAX7 in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of PAX7 in the tissuegenic cell (e.g., myogenic cell) after about 72 hours, about 84 hours, about 96 hours or longer, including any value or range in between these values. In some embodiments, the level of expression and/or activity of PAX7 increases by at least any one of 10%, 20%, 50%, 2×, 3×, 5×, 10× or more, including any value or range in between these values.

In some embodiments, the one or more FAO activators increases expression and/or activity of MyoD (e.g., MyoD1) in a tissuegenic cell (e.g., myogenic cell, e.g., myoblast or myocyte). In some embodiments, the one or more FAO activators increases the expression and/or activity of MyoD in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of MyoD in the tissuegenic cell (e.g., myogenic cell) after about 72 hours, about 84 hours, about 96 hours or longer, including any value or range in between these values. In some embodiments, the level of expression and/or activity of MyoD increases by at least any one of 10%, 20%, 50%, 2×, 3×, 5×, 10× or more, including any value or range in between these values.

In some embodiments, the one or more FAO activators increases expression and/or activity of MyoG in a tissuegenic cell (e.g., myogenic cell, e.g., myoblast or myocyte). In some embodiments, the one or more FAO activators increases the expression and/or activity of MyoG in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of MyoG in the tissuegenic cell (e.g., myogenic cell) after about 72 hours, about 84 hours, about 96 hours or longer, including any value or range in between these values. In some embodiments, the level of expression and/or activity of MyoG increases by at least any one of 10%, 20%, 50%, 2×, 3×, 5×, 10× or more, including any value or range in between these values.

In some embodiments, the one or more FAO activators increases expression and/or activity of Myh3 in a tissuegenic cell (e.g., myogenic cell, such as myoblast or myocyte). In some embodiments, the one or more FAO activators increases the expression and/or activity of Myh3 in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of Myh3 in the tissuegenic cell (e.g., myogenic cell) after about 72 hours, about 84 hours, about 96 hours or longer, including any value or range in between these values. In some embodiments, the level of expression and/or activity of Myh3 increases by at least any one of 10%, 20%, 50%, 2×, 3×, 5×, 10× or more, including any value or range in between these values.

In some embodiments, the one or more FAO activators increases expression and/or activity of PPARγ in a tissuegenic cell (e.g., myogenic cell, such as myoblast or myocyte). In some embodiments, the one or more FAO activators increases the expression and/or activity of PPARγ in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of PPARγ in the tissuegenic cell (e.g., myogenic cell) after about 72 hours, about 84 hours, about 96 hours or longer, including any value or range in between these values. In some embodiments, the level of expression and/or activity of PPARγ increases by at least any one of 10%, 20%, 50%, 2×, 3×, 5×, 10× or more, including any value or range in between these values.

In some embodiments, the one or more FAO activators increases expression and/or activity of PPARα in a tissuegenic cell (e.g., myogenic cell, such as myoblast or myocyte). In some embodiments, the one or more FAO activators increases the expression and/or activity of PPARα in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of PPARα in the tissuegenic cell (e.g., myogenic cell) after about 72 hours, about 84 hours, about 96 hours or longer, including any value or range in between these values. In some embodiments, the level of expression and/or activity of PPARα increases by at least any one of 10%, 20%, 50%, 2×, 3×, 5×, 10× or more, including any value or range in between these values.

In some embodiments, the one or more FAO activators increases level of H3K9ac (acetylated histone H3 lysine 9) in a tissuegenic cell (e.g., myogenic cell, such as myoblast or myocyte). In some embodiments, the one or more FAO activators increases the level of H3K9ac in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the level of H3K9ac in the tissuegenic cell (e.g., myogenic cell) after about 72 hours, about 84 hours, about 96 hours or longer, including any value or range in between these values. In some embodiments, the level of H3K9ac increases by at least any one of 10%, 20%, 50%, 2×, 3×, 5×, 10× or more, including any value or range in between these values.

In some embodiments, the one or more FAO activators increases expression and/or activity of Ki67 in a tissuegenic cell (e.g., myogenic cell, such as myoblast or myocyte). In some embodiments, the one or more FAO activators increases the expression and/or activity of Ki67 in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not increase the expression and/or activity of Ki67 in the tissuegenic cell (e.g., myogenic cell) after about 72 hours, about 84 hours, about 96 hours or longer, including any value or range in between these values. In some embodiments, the level of expression and/or activity of Ki67 increases by at least any one of 10%, 20%, 50%, 2×, 3×, 5×, 10× or more, including any value or range in between these values.

In some embodiments, the one or more FAO activators upregulates one or more genes in the FAO and/or lipid metabolism pathways in a tissuegenic cell (e.g., myogenic cell, such as myoblast or myocyte). In some embodiments, the one or more FAO activators upregulates one or more genes in the FAO and/or lipid metabolism pathways in the tissuegenic cell (e.g., myogenic cell) for about 12 hours, about 24 hours, about 36 hours, about 48 hours, about 60 hours, or about 72 hours, including any value or range in between these values. In some embodiments, the one or more FAO activators does not upregulate one or more genes in the FAO and/or lipid metabolism pathways in the tissuegenic cell (e.g., myogenic cell) after about 72 hours, about 84 hours, about 96 hours or longer, including any value or range in between these values. In some embodiments, the level of expression and/or activity of one or more genes in the FAO and/or lipid metabolism pathways increases by at least any one of 10%, 20%, 50%, 2×, 3×, 5×, 10× or more, including any value or range in between these values. In some embodiments, the one or more FAO activators upregulates one or more FAO and lipid metabolism genes including but not limited to the nuclear hormone receptors PPARA, PPARD, PPARG, RXRB, RXRG, NCOA1, NCOA2; the upstream fatty acid transporters FABP3, FABP4, CD36, SCARB1, FATP1-6; a variety of lipases including LPL; the rate-limiting carnitine palmitoyl-transferases CPT1A and CPT1B; the carnitine acetylase CRAT; the acyl-CoA dehydrogenases ACADs and hydroxyacyl-CoA dehydrogenases HADHs; and the mitochondrial electron transfer flavoproteins ETFA and ETFB, which can promote myogenic differentiation.

The expression and/or activity of PAX7, MyoD, MyoG, Myh3, PPARγ, PPARα, H3K9ac, and genes in the FAO and lipid metabolism pathways can be determined using any known methods in the art, for example, by quantitative reverse-transcription PCR, immunostaining, microarray, RNA sequencing, Western blot, as well as metabolomics and lipidomics analysis.

In some embodiments, the one or more FAO activators comprises an activator of a gene selected from the group consisting of transcriptional regulators of lipid metabolism, fatty acid transporters, lipases, carnitine palmitoyl-transferases, carnitine acetylase, acyl-CoA dehydrogenases, hydroxyacyl-CoA dehydrogenases, and the mitochondrial electron transfer flavoproteins.

In some embodiments, the one or more FAO activators comprises an activator of a gene selected from the group consisting of PPARα, PPARδ, PPARγ, RXRB, RXRG, NCOA1, NCOA2, FABP3, FABP4, CD36, SCARB1, FATP1, FATP2, FATP3, FATP4, FATP5, FATP6, LPL, CPT1A, CPT1B, CPT1C, CPT2, CRAT, ACADs (e.g., ACAD1, ACAD2, ACAD3, ACAD4, ACAD5, ACAD6, ACAD7, ACAD8, ACAD9, ACAD10, ACAD11, MCAD, LCAD, VLCAD), HADHs (e.g., HADHA, HADHB), ETFA and ETFB.

In some embodiments, the one or more FAO activators (such as PPARγ agonist e.g., rosiglitazone and/or prostaglandin e.g., PGI2, PGD2 or analogue thereof) is in a pharmaceutical composition. The pharmaceutical composition may be formulated for a suitable route of delivery, such as oral, parenteral, rectal, nasal, topical, or by inhalation. Preferably, the compositions are formulated for intramuscular, subcutaneous, or oral administration.

In some embodiments, the one or more FAO activators comprises one or more activators of PPAR. In some embodiments, the one or more FAO activators comprises one or more activators of PPARγ. In some embodiments, the one or more FAO activators is an activator of PPAR. In some embodiments, the one or more FAO activators is an activator of PPARγ.

Any suitable activators of PPARγ may be used in the methods described herein. In some embodiments, the one or more activators of PPARγ increases the expression of PPARγ. In some embodiments, the one or more activators of PPARγ increases the activity of PPARγ. In some embodiments, the activator of PPARγ is a nucleic acid (e.g., mRNA) encoding PPARγ. In some embodiments, the activator of PPARγ is a miRNA that increases the expression of PPARγ. In some embodiments, the activator of PPARγ is a PPARγ agonist. In some embodiments, the activator of PPARγ is a prostaglandin selected from the group consisting of PGI2, PGD2, analogues thereof, and salts, solvates, tautomers, and stereoisomers thereof.

In some embodiments, the one or more FAO activators comprises a PPARγ agonist and a prostaglandin selected from the group consisting of PGI2, PGD2, and analogues thereof. In some embodiments, the one or more FAO activators is a combination of a PPARγ agonist and PGI2. In some embodiments, the one or more FAO activators is a combination of a PPARγ agonist and an analogue of PGI2. In some embodiments, the one or more FAO activators is a combination of a PPARγ agonist and PGD2.

### PPARγ Agonists

PPARγ agonists are known in the art. Suitable examples of PPARγ agonists useful for the methods described herein include thiazolidine (“TZD”) derivatives known as thiazolidinediones. Exemplary thiazolidinediones include but are not limited torosiglitazone, pioglitazone, proglitazone, troglitazone, C1-991 (Parke-Davis), BRL 49653, ciglitazone, englitazone and chemical derivatives thereof. These compounds are conventionally known for the treatment of diabetes. See, e.g., U.S. Pat. Nos. 4,812,570; 4,775,687; 4,725,610; 4,582,839; and 4,572,912 for exemplary sources of such compounds. U.S. Pat. No. 5,521,201 and European Patent Applications 0008203, 0139421, 0155845, 0177353, 0193256, 0207581 and 0208420, and Chem. Pharm. Bull 30 (10) 3580-3600 relate to thiazolidinedione derivatives, and describe commercial sources/synthetic schemes for a variety of TZD and TZD-like analogues, which may be useful in carrying out the method of the present application. Another exemplary PPAR agonist is a compound of the glitazar class, such as aleglitazar, muraglitazar, tesaglitazar, ragaglitazar and saroglitazar. Non-thiazolidinedione PPARγ agonists, such as GW2570, elafibranor, WY-14643 (pirinixic acid), bisphenol A diglycidyl ether (BADGE), L-796,449, GW1929, T33, INT131, FK614, 2-(2-(4-phenoxy-2-propylphenoxy)ethyl)indole-5-acetic acid, efatutazone, 15d-PGJ2,9- and 13-hydroxyoctadecanoic acid, PGI2 (prostacyclin) and prostacyclin analogues such as treprostinil, carbacyclin, isocarbacyclin, iloprost (ciloprost), cicaprost, cisaprost, beraprost and epoprostenol may also be used in the methods described herein. The PPARγ agonists contemplated herein include pharmaceutically acceptable salts, solvates, tautomers, stereoisomers, prodrugs, and combinations of suitable PPARγ agonist compounds known in the art.

In some embodiments, the PPARγ agonist is a compound of Formula (I):

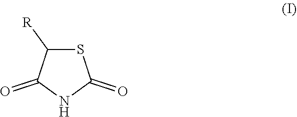

or a salt, solvate, tautomer, or stereoisomer thereof, wherein R is selected from the group consisting of hydrogen, unsubstituted and substituted C₁₋₆alkyl, unsubstituted and substituted C₂₋₆alkenyl, unsubstituted and substituted C₂₋₆alkynyl, unsubstituted and substituted aryl, unsubstituted and substituted heteroaryl, and unsubstituted and substituted heterocyclyl.

In some embodiments, the PPARγ agonist is a compound of Formula (II):

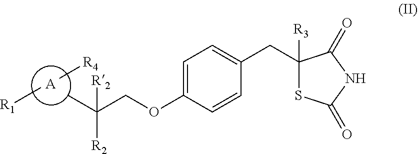

or a salt, solvate, tautomer, or stereoisomer thereof, wherein each of R₁ and R₄ is independently selected from the group consisting of hydrogen, halo, unsubstituted alkyl, alkyl substituted with 1-3 of halo, unsubstituted alkoxy, and alkoxy substituted with 1-3 of halo; wherein R₂ is selected from the group consisting of halo, hydroxy, unsubstituted and substituted alkyl; wherein R′₂ is hydrogen, or R₂ and R′₂ together form oxo; wherein R₃ is H; and wherein Ring A is a phenyl.

In some embodiments, the PPARγ agonist is rosiglitazone, or a salt, solvate, tautomer, or stereoisomer thereof.

In some embodiments, the PPARγ agonist is a compound of Formula (III):

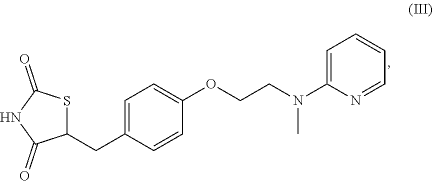 [SMILES: CN(CCOc1ccc(CC2SC(=O)NC2=O)cc1)c1ccccn1]

or a salt, solvate, tautomer, or stereoisomer thereof.

### Prostaglandins

In some embodiments, the one or more FAO activators comprises a prostaglandin that activates PPARγ, including naturally occurring prostaglandins, analogues thereof, salts, solvates, tautomers, and stereoisomers thereof. Exemplary prostaglandins that activates PPARγ include, but are not limited to, PGI2 and PGD2.

PGI2 is also known as prostacyclin. It is a prostaglandin member of the eicosanoid family of lipid molecules. When used as a drug, PGI2 is known as epoprostenol, which is used to treat pulmonary arterial hypertension.

In some embodiments, the one or more FAO activators comprises (or is) a compound of Formula (IV):

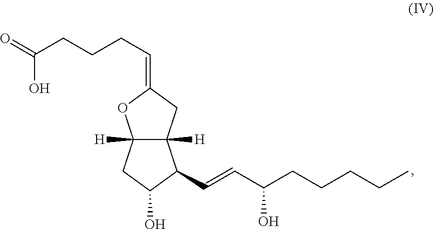 [SMILES: CCCCC[C@H](O)/C=C/[C@@H]1[C@H]2C/C(=C/CCCC(=O)O)O[C@H]2C[C@H]1O]

or a salt, solvate, tautomer, or stereoisomer thereof.

In some embodiments, the one or more FAO activators comprises an analogue of PGI2. PGI2 analogues are known in the art, including, but not limited to iloprost and treprostinil. In some embodiments, the PGI2 analogue is treprostinil, or a salt, solvate, tautomer, or stereoisomer thereof. In some embodiments, the PGI2 analogue is treprostinil sodium.

In some embodiments, the one or more FAO activators comprises (or is) a compound of Formula (V):

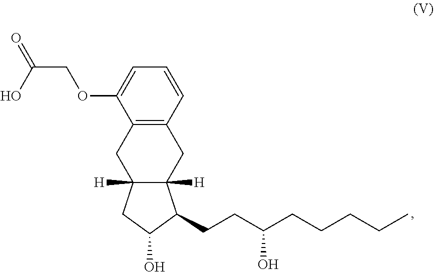 [SMILES: CCCCC[C@H](O)CC[C@@H]1[C@H]2Cc3cccc(OCC(=O)O)c3C[C@H]2C[C@H]1O]

or a salt, solvate, tautomer, or stereoisomer thereof.

PGD2 is a prostaglandin that binds to the receptor PTGDR (DP1), as well as CRTH2 (DP2). In some embodiments, the one or more FAO activators comprises (or is) a compound of Formula VI):

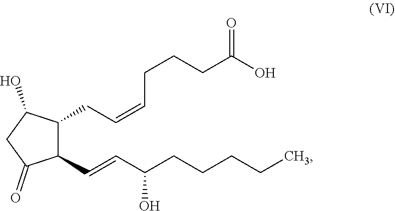 [SMILES: CCCCC[C@H](O)/C=C/[C@H]1C(=O)C[C@H](O)[C@@H]1C/C=C\CCCC(=O)O]

or a salt, solvate, tautomer, or stereoisomer thereof.

In some embodiments, the one or more FAO activators comprises an analogue of PGD2. In some embodiments, the analogue of PDG2 is a compound of Formula (VII):

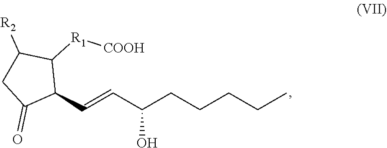

or a salt, solvate, tautomer, or stereoisomer thereof, wherein each of R₁ and R₂ is selected from the group consisting of halo, hydroxy, unsubstituted and substituted alkyl.

In some embodiments, the one or more FAO activators is a combination of rosiglitazone and PGI2. In some embodiments, the one or more FAO activators is a combination of rosiglitazone and treprostinil. In some embodiments, the one or more FAO activators is a combination of rosiglitazone and PGD2.

In some embodiments, the one or more FAO activators is a combination of pirinixic acid (WY-14643) and PGI2. In some embodiments, the one or more FAO activators is a combination of pirinixic acid (WY-14643) and PGD2. In some embodiments, the one or more FAO activators is a combination of pirinixic acid (WY-14643) and treprostinil.

### V. Kits and Articles of Manufacture

The present application further provides kits, formulations, unit dosages, and articles of manufacture for use in any one of the methods of muscle regeneration in vitro or in vivo, and methods of treatment described herein.

In some embodiments, there is provided a kit for promoting myogenesis and/or inducing differentiation and/or maturation of a tissuegenic cell (e.g., myogenic cell such as myoblast or myocyte), comprising one or more FAO activators such as a PPARγ agonist (e.g., rosiglitazone) and/or PGI2 or analogue thereof (e.g., treprostinil). In some embodiments, the kit is useful for in vitro cell culture. In some embodiments, the kit is useful for ex vivo culture of tissuegenic cells (e.g., myogenic cells). In some embodiments, the kit is useful for in vivo application.

In some embodiments, there is provided a kit for treating a muscle disease or condition (e.g., muscle injury or muscle degeneration) in an individual, comprising a pharmaceutical composition comprising one or more FAO activators such as a PPARγ agonist (e.g., rosiglitazone) and/or PGI2 or analogue thereof (e.g., treprostinil). In some embodiments, the kit further comprises tissuegenic cells (e.g., myogenic cells, such as myoblasts and/or myocytes).

The kit may contain additional components, such as containers, reagents, culturing media, buffers, and the like to facilitate execution of any embodiment of the methods. For example, in some embodiments, the kit further comprises a cell collection and storage apparatus, which can be used to collect an individual's tissuegenic cells (e.g., myogenic cells, such as myoblasts). In some embodiments, the kit further comprises culturing mediator containers (e.g., petri dishes and plates) for proliferation and/or differentiation of tissuegenic cells (e.g., myogenic cells). In some embodiments, the kit further comprises immunostaining or histology reagents for assessing biomarkers of the tissuegenic cells (e.g., myogenic cells).

The kits of the present application are in suitable packaging. Suitable packaging include, but is not limited to, vials, bottles, jars, flexible packaging (e.g., Mylar or plastic bags), and the like. Kits may optionally provide additional components such as interpretative information. The present application thus also provides articles of manufacture, which include vials (such as sealed vials), bottles, jars, flexible packaging, and the like.

The kits may also comprise instructions relating to the use of the one or more FAO activators in any one of the methods described herein. In some embodiments, the kit further comprises an instructional manual, such as a manual describing a protocol according to any one of the methods of muscle regeneration, or methods of treatment described herein. The instructions may also include information on dosage, dosing schedule, and routes of administration of the one or more FAO activators or tissuegenic cells (e.g., myogenic cells) using the kit for the intended treatment.

Also provided are unit dosage forms comprising the one or more FAO activators and formulations described herein. These unit dosage forms can be stored in a suitable packaging in single or multiple unit dosages and may also be further sterilized and sealed. In some embodiments, the composition (such as pharmaceutical composition) is contained in a single-use vial, such as a single-use sealed vial. In some embodiments, the composition (such as pharmaceutical composition) is contained in a multi-use vial. In some embodiments, the composition (such as pharmaceutical composition) is contained in bulk in a container.

### EXAMPLES

The examples below are intended to be purely exemplary of the present application and should therefore not be considered to limit the invention in any way. The following examples and detailed description are offered by way of illustration and not by way of limitation.

### Example 1: an Early Transient Burst of PPAR-Driven Fatty Acid Oxidation Enhances Tissue Regeneration

By carefully mapping metabolomic profiles during the earliest phases of primary human myoblast differentiation, we uncovered a transient burst in mitochondrial fatty acid oxidation (FAO) and redox stress during the transition from proliferative primary myoblasts into non-proliferative myocytes. In addition, we document that this burst of FAO is specific to the early stages of differentiation, and it is associated with a transient increase in mitochondrial oxygen consumption, without significant changes in mitochondrial biogenesis or membrane potential. Mechanistically, the early burst of mitochondrial FAO is regulated by the transient rise of MyoD, PPARγ and PPARα to promote the early cellular differentiation program in human myocytes. We found that the PPARγ-FAO axis is pro-myogenic only in the early stages of differentiation, but anti-myogenic in the later stages of differentiation. In vivo, we found that early transient treatment with the anti-diabetic PPARγ agonist rosiglitazone can enhance mouse skeletal muscle regeneration by transiently increasing FAO flux, with important implications for our understanding of how exercise and nutrition can be regulated to influence muscle regeneration and degeneration.

### Materials and Methods

Gene Expression Omnibus database mining

We mined transcriptomic data on human myoblast differentiation (GSE55034) in the Gene Expression Omnibus (GEO) database and analyzed them using the R Bioconductor package.

### Metabolomics and Lipidomics Analyses

Liquid chromatography-tandem mass spectrometry (LC-MS/MS) metabolomics and lipidomics analyses were performed according to previously published protocols (Chong et al., 2012).

### MALDI-MS Imaging Analysis

All MSI experiments were performance using an MALDI-FT-ICR instrument (solariX 9.4T, Bruker Daltonics) equipped with a smartbeam laser (Bruker Daltonics) in the mass range of m/z 100-1000 in positive ion mode at a frequency of 1000 Hz, using 200 laser shots and a spatial resolution of 100 μm. All data were processed using the FlexImaging 3.0 software (Bruker Daltonics).

### Cell Culture and Drug Treatment

Primary human skeletal muscle (HSKM) progenitors (Gibco) were cultured on gelatin solution (0.1%, Merck-Millipore)-coated plate and incubated in a humidified atmosphere (5% CO₂ and 37° C.) with growth medium composed of DMEM/F-12 (Gibco) supplemented with fetal bovine serum (FBS) (20%, GE Healthcare), L-glutamine (1%, Gibco) and penicillin-streptomycin (1%, Gibco). Confluent HSKM progenitors were induced to differentiate by replacing growth media with differentiation medium, comprising of DMEM/F-12, KnockOut Serum Replacement (2%, Gibco), L-glutamine (1%, Gibco) and penicillin-streptomycin (1%, Gibco). For drug treatments (all from Cayman Chemical), cells were incubated with etomoxir (10 uM), rosiglitazone (10 M), GW6471 (0.1 M), GSK3787 (1 uM), PGI2 (10 ng/ml), treprostinil (1 nM), and GW9662 (0.1 μM). MitoTracker Red (200 nM, Thermo Fisher) and JC1 (2 uM, Thermo Fisher) staining were performed according to manufacturer's instructions and stained cells were imaged with a Zeiss fluorescence microscope.

### Transfection of HSKM Progenitors Using Polyethylenimine

HSKM progenitors were seeded onto gelatin-coated plates and cultured in growth media. One day after seeding, a mixture of polyethylenimine (PEI)/RNA was used to transfect the HSKM cells. To prepare the mixtures, PEI together with the following RNAs were mixed with serum-free DMEM, hsa-let-7 miRCURY LNA microRNA Power Family Inhibitor (YFI0450006, Qiagen), a combination of mirVana miRNA mimic hsa-let-7a-5p (4464066, Assay ID: MC10050, Thermo Fisher) and mirVana miRNA mimic hsa-let-7b-5p (4464066, Assay ID: MCi 1050, Thermo Fisher), MYOD1 siRNA (4392420, siRNA ID: s9231, Thermo Fisher), MLYCD siRNA TriFECTa DsiRNA Kit (Design ID: hs.Ri.MLYCD.13, IDT) or Cy5-conjugated scramble RNAi control. The PEI/RNA transfection mixtures were incubated at room temperature for 20 mins before being added to the HSKM cells. The transfection media was replaced with growth media after 24 hours.

### Quantitative PCR

RNA was extracted using TRIzol (Thermo Fisher) and reverse transcribed to cDNA with Superscript III (Thermo Fisher) according to manufacturer's instructions. The synthesized cDNA was diluted 5× in H2O before performing qPCR with KAPA SYBR FAST (Merck) on ABI Prism 7900HT (Applied Biosystems) real-time PCR system according to manufacturers' instructions. Primer sequences are provided in Table 1.

TABLE 1

| | | SEQ | | SEQ |
|---|---|---|---|---|
| Human | | ID | | ID |
| Gene | Forward 5′ to 3′ | NO | Reverse 5′ to 3′ | NO |
| GAPDH | TGGTATCGTGGAAGGACTCA | 1 | TTCAGCTCAGGGATGACCTT | 12 |
| MYOD1 | CGGCATGATGGACTACAGCG | 2 | CAGGCAGTCTAGGCTCGAC | 13 |
| MYOG | GGGGAAAACTACCTGCCTGTC | 3 | AGGCGCTCGATGTACTGGAT | 14 |
| MYHC | TTCATTGGGGTCTTGGACAT | 4 | AACGTCCACTCAATGCCTTC | 15 |
| MYH3 | ATTGCTTCGTGGTGGACTCAA | 5 | GGCCATGTCTTCGATCCTGTC | 16 |
| MYH7 | TGCCACATCTTGATCTGCTC | 6 | CTCGGCTTCAAGGAAAATTG | 17 |
| MYH8 | TAAACACACCTGCCTGATGC | 7 | TCAGCTTTAACAGGAAAATAAACG | 18 |
| SkActA | CGACATCAGGAAGGACCTGTATGCC | 8 | GGCCTCGTCGTACTCCTGCTTGG | 19 |
| PPARA | TCGGCGAGGATAGTTCTGGAAG | 9 | GACCACAGGATAAGTCACCGAG | 20 |
| PPARD | GGCTTCCACTACGGTGTTCATG | 10 | CTGGCACTTGTTGCGGTTCTTC | 21 |
| PPARG | AGCCTGCGAAAGCCTTTTGGTG | 11 | GGCTTCACATTCAGCAAACCTGG | 22 |

miRNA Quantitative PCR

After HSKM progenitors were induced to differentiate, HSKM cells were harvested every 12 hourly for 84 hours. Cold TRIzol (Thermo Fisher) reagent was added onto the HSKM cells and the cell lysate stored at −30 C, until all the samples were available for RNA isolation. The isolated RNA samples were reverse transcribed and amplified by miScript RT Kit (Qiagen) according to manufacturer's instructions. miRNA quantitative PCR was performed using the miScript SYBR Green PCR kit (Qiagen) on ABI Prism 7900HT (Applied Biosystems) real-time PCR system according to manufacturers' instructions. The following miScript Primer Assays (Qiagen) were used, Hs_let-7a_2 (MS00031220), Hs_let-7b_1 (MS00003122), Hs_let-7e_3 (MS00031227) and Hs_let-7g_2 (MS00008337).

### Mitochondrial DNA Copy Number Measurement

After HSKM progenitors were induced to differentiate, HSKM cells were harvested every 12 hourly for 84 hours. Genomic DNA was isolated from HSKM cells using the DNeasy Blood & Tissue Kit (Qiagen) according to manufacturer's instructions. Briefly, HSKM cells were washed with phosphate buffered saline (PBS) (Thermo Fisher), trypsinized (0.25%, Thermo Fisher) at 37° C. for 3 min and centrifuged at 1300 rpm for 3 min. The harvested cell pellets were subsequently stored at −80° C., until all the samples were available for DNA isolation. For mitochondrial DNA copy number measurement, qPCR-based mitochondrial quantification was performed using KAPA SYBR FAST (Merck) on ABI Prism 7900HT (Applied Biosystems) real-time PCR system according to manufacturers' instructions. Primer sequences are provided in Table 2.

TABLE 2

| | | SEQ | | SEQ |
|---|---|---|---|---|
| Human | | ID | | ID |
| gene | Forward 5′ to 3′ | NO | Reverse 5′ to 3′ | NO |
| B2M | CACTGAAAAAGATGAGTATGCC | 23 | AACATTCCCTGACAATCCC | 29 |
| ND1 | ACGCCATAAAACTCTTCACCAAAG | 24 | GGGTTCATAGTAGAAGAGCGATGG | 30 |
| ND4 | ACCTTGGCTATCATCACCCGAT | 25 | AGTGCGATGAGTAGGGGAAGG | 31 |
| ND5 | AGTTACAATCGGCATCAACCAA | 26 | CCCGGAGCACATAAATAGTATGG | 32 |
| ND6 | TGGGGTTAGCGATGGAGGTAGG | 27 | AATAGGATCCTCCCGAATCAAC | 33 |
| Mito | CACTTTCCACACAGACATCA | 28 | TGGTTAGGCTGGTGTTAGGG | 34 |

### Oxygen Consumption Analyses

HSKM progenitors were seeded onto Seahorse XF96 Cell Culture Microplate (Agilent), pre-coated with gelatin (0.1%, Merck-Millipore), in growth media at 10,000 cells per well. 2 days after seeding, HSKM progenitors were induced to differentiate by replacing growth media with differentiation medium. Before performing the Seahorse XF cell Mito Stress Test assay, cell culture media were replaced with assay media (Seahorse XF DMEM Medium, pH 7.4, 2 mM pyruvate, 2 mM glutamine) (Agilent) and incubated in a CO₂-free incubator at 37° C. for 1 hour to equilibrate temperature and pH for each well. During the assay, Oligomycin (2 uM, Agilent), FCCP (0.5 uM, Agilent), and a mixture of Antimycin A and Rotenone (0.5 uM, Agilent), were injected sequentially and measurements were taken according to manufacturer's instructions. The data was analyzed using WAVE software.

### Western Blot

Protein was extracted with RIPA buffer (Thermo Fisher) supplemented with protease inhibitor cocktails I and II (Merck) and phosphatase inhibitor cocktail set III (Merck). Protein was quantified with Pierce BCA protein assay kit (Thermo Fisher) and analyzed with Sunrise Tecan plate reader. After SDS-PAGE and electro-transfer onto PVDF membranes (GE Healthcare), western blot was performed with the following primary antibodies, MyoD (1:50, sc-760, Santa Cruz Biotechnology), MyoG (1:200, sc-576, Santa Cruz Biotechnology), myosin heavy chain MHC eFluor 660 (1:20, 50-6503-82, Thermo Fisher), α-Actinin (Sarcomeric) (1:500, A7811, Merck), PPARA (1:1000, CST), PPARD (1:1000, CST), PPARG (1:1000, 2443S, CST), H3K9ac (1:1000, CST), and GAPDH (1:1000, sc-25778, Santa Cruz Biotechnology). Subsequently, blots were stained with secondary antibody anti-rabbit IgG HRP conjugate (1:2500, W401B, Promega) and anti-mouse IgG HRP conjugate (1:2500, W402B, Promega). Protein levels were detected using ECL prime western blotting detection reagent kit (GE Healthcare).

### Immunofluorescence

Cells were washed with PBS (Thermo Fisher) and fixed with paraformaldehyde (PFA) (4%, Electron Microscopy Sciences) at room temperature for 10 mins. Cells were stained with primary antibody myosin heavy chain eFluor 660 (1:20, 50-6503-82, Thermo Fisher) at 4 C overnight. DAPI (Merck) was used as a nuclear counterstain according to manufacturer's recommendations. Stained cells were imaged with a Zeiss fluorescence microscope.

### Muscle Cryo-Injury

Eight week old NOD scid gamma (NSG) or C57BL/6 mice were anaesthetized with a mixture of ketamine and xylazine (120 mg/kg and 8 mg/kg respectively) via intraperitoneal injection. After successful anesthetization, the skin over the tibialis anterior (TA) or gastrocnemius or quadriceps muscle was disinfected by wiping with 70% ethanol and a 3 mm incision was made over the TA muscle. A dry-ice-chilled 4-mm metal probe was directly applied onto the exposed skeletal muscle for three cycles of five seconds to induce cryo-injury. Thereafter, the incision was immediately sutured using a surgical suture stapler. Upon recovery under heat lamps for a period of 2 hours, the mice were randomly allocated to each treatment groups. All the drugs (rosiglitazone (20 mg/kg), etomoxir (20 mg/kg), GW0742 (1 mg/kg), GSK3787 (5 mg/kg), fenofibrate (30 mg/kg), WY-14643 (30 mg/kg), PGI2 (3.2 mM), PGF1a (3.2 mM), PGD2 (3.2 mM), PGG1 (3.2 mM), treprostinil (1 mM), HGF (4 ng/uL) and DMSO vehicle (all Cayman Chemical) were intramuscularly injected into the TA muscle using an insulin syringe (BD). At the end of the experiment, muscular strength was measured using the grip strength meter (Bioseb). Seven days to twenty-seven days after cryo-injury, the TA muscles were biopsied or harvested for histology and western blot. For the histology samples, TA muscles were incubated in 4% PFA solution overnight and embedded in paraffin. Samples were serially sectioned until depleted and haemotoxylin and eosin (H&E) staining was performed on every 12th 5 um-thick tissue section. After microscopy imaging, the area of the cryo-injured myofibers was quantified using ImageJ. For western blot samples, TA muscles were snap-frozen in liquid nitrogen and homogenized in RIPA buffer (Thermo Fisher) supplemented with protease inhibitor cocktails I and II (Merck) and phosphatase inhibitor cocktail set III (Merck) using TissueLyser II (Qiagen).

### Intramuscular Injection of GFP-Positive HSKM Cells

Lentiviral eGFP expression vector pLenti CMV GFP Blast (659-1) (Addgene #17445) was packaged into lentiviral particles. To obtain GFP-positive HSKM progenitors, cells were then transduced with the viral particles and selected with growth media containing blasticidin (25 ug/ml, InvivoGen) for 5-7 days. Cryo-injury was carried out on eight-week-old NSG mice as mentioned above and subsequently mice were randomly allocated into 2 groups for HSKM transplantation, rosiglitazone-treated GFP-positive HSKM and DMSO-treated GFP-positive HSKM. GFP-positive HSKM were treated with growth media containing rosiglitazone or DMSO control for 24 hr and trypsinized for cell transplantation. 2 million HSKM cells were resuspended in 100 ul of growth media containing Matrigel hESC-Qualified Matrix (1:1, Corning). Using a 23-gauge needle, the cell suspension was injected into the TA muscle. 7 days after cryo-injury, the TA muscles were harvested in 4% PFA overnight and embedded in paraffin.

### Immunohistochemistry

Tibialis anterior (TA) tissue samples embedded in paraffin were sectioned using a microtome and transferred onto Leica Microsystems Plus Slides. Some tissues were flash frozen for cryosectioning. Paraffin-embedded sections were deparaffinized in xylene (Merck) for 2 washes (10 mins) and then transferred sequentially into 100% EtOH (Merck), 100% EtOH, 95% EtOH and 70% EtOH (2 mins) at room temperature. The sections were then rehydrated in deionized water (3 mins). Antigen retrieval was carried out using the 2100 Retriever in sodium citrate buffer (Merck, pH 6.2, 30 mins). Slides were then cooled in cold PBS (15 mins) and blocked in blocking buffer at room temperature (30 mins). Primary antibody staining was conducted in blocking buffer at 4° C. overnight with the following antibodies, GFP (1:500, sc-9996, Santa Cruz), Pax7 (5 ug/ml, 042349, DSHB), MyoD (5 ug/ml, sc-377460, Santa Cruz), Ki67 (1:100, 14-5698-82, Thermo Fisher), embryonic MHC (Myh3; 1:100, sc-53091, Santa Cruz), PDGFRa (5 ug/mL, AF1062, R&D), F4/80 (1:100, ab6640, Abcam), PPARG (1:1000, 2443S, CST), and myosin heavy chain eFluor 660 (1:20, 50-6503-82, eBioscience). After the slides were washed thrice in PBS (10 mins) and counterstained with DAPI, secondary antibody staining for GFP was conducted in blocking buffer at room temperature (1 hr) with goat anti-mouse IgG secondary antibody, Alexa Fluor 488 (1:500, A11001, Thermo Fisher).

### Results

### Metabolomic Analysis of Early Primary Human Myoblast Differentiation

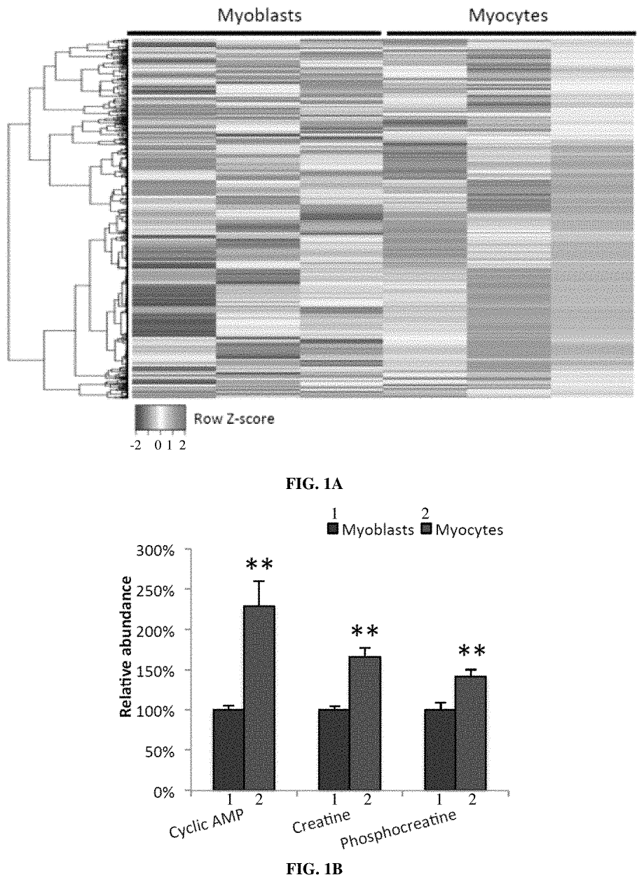

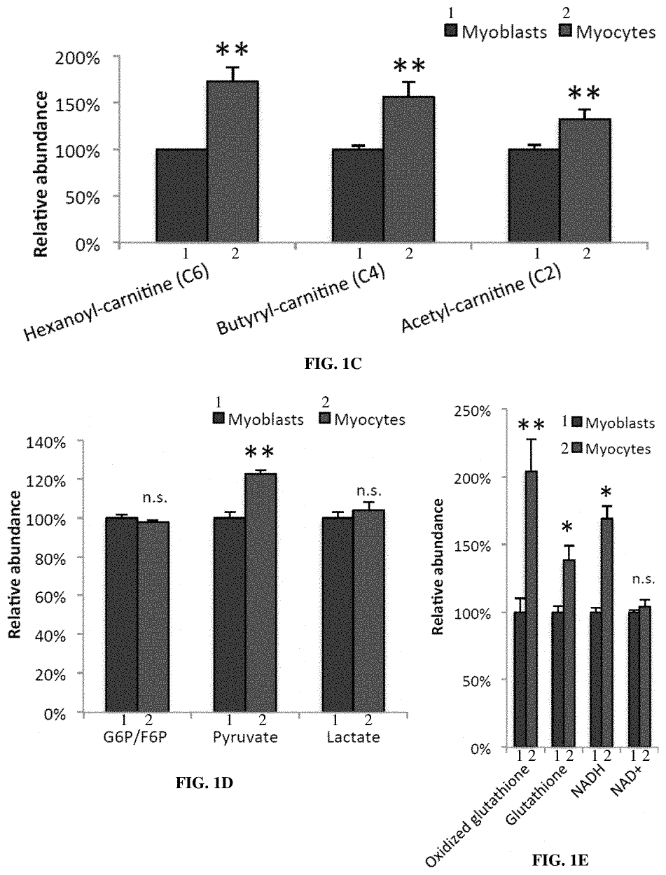

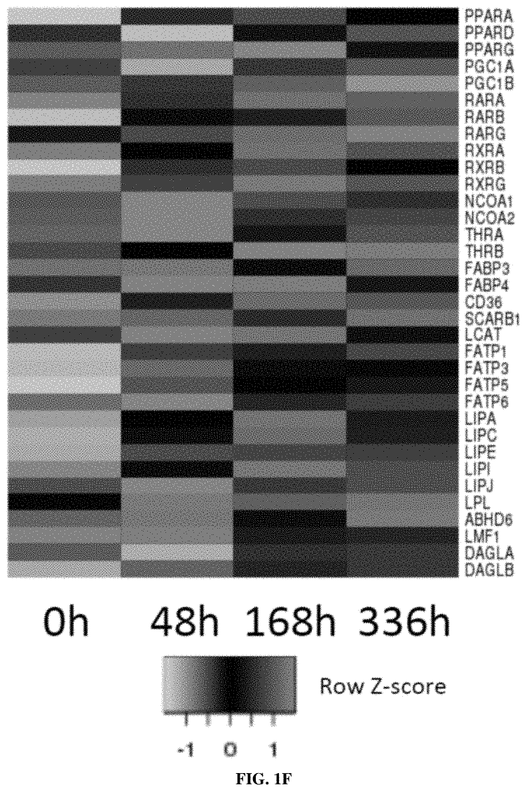

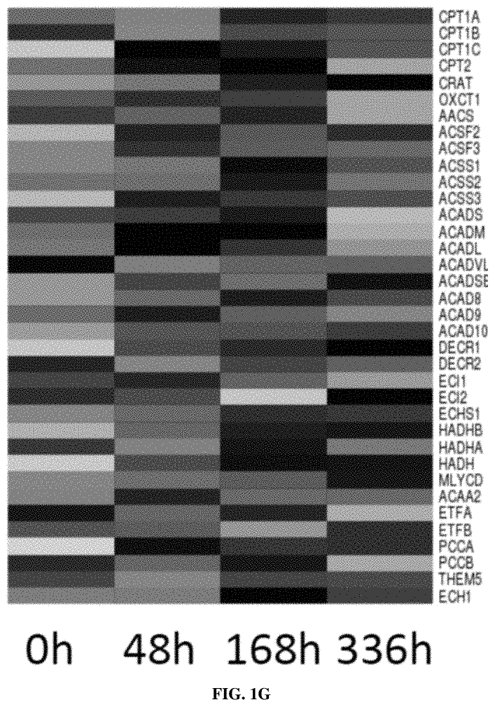

To globally survey the metabolic changes that are induced during the earliest stages of myoblast differentiation, we performed LC-MS/MS metabolomics profiling of primary human myoblasts and myocytes. Primary human myoblasts were subjected to serum withdrawal conditions for 48 hours to halt proliferation and induce cellular differentiation to generate primary human myocytes. Serum withdrawal induced critical changes in the metabolome of primary human myoblasts as they underwent a phase transition to differentiate into non-proliferative myocytes (FIG. 1A). As expected of myocytes that are in the early phase of myogenic differentiation, when they activate PKA signalling and muscle creatine kinase (Naro et al., 2003), we observed significant increases in cyclic AMP, creatine and phosphocreatine (FIG. 1B). Coinciding with these myocyte-specific metabolic changes, we observed significant increases in short chain acyl-carnitines and acetyl-carnitine (FIG. 1C), which are intermediates of mitochondrial β-oxidation or fatty acid oxidation (FAO). In contrast no significant decreases were observed with the glycolytic intermediates yet, including lactate production, at this early phase of myogenic differentiation (FIG. 1D).

Redox-related metabolites increased contemporaneously with the increase in FAO intermediates, including oxidized glutathione, glutathione, and NADH (FIG. 1E). Comparing the oxidized glutathione/reduced glutathione ratio, and the NADH/NAD⁺ ratio, our results suggested an increase in both oxidative stress and reducing power. This is consistent with an increase in mitochondrial FAO flux during the earliest phase of myoblast differentiation, since mitochondrial FAO is well-known to efficiently increase NADH and reactive oxygen species (ROS) production.

To examine if the increased FAO in early myogenic differentiation is supported by transcriptional or post-transcriptional changes, we mined transcriptomic data on primary human myoblast differentiation in the GEO database (GSE55034). We found that a variety of lipid metabolism and FAO-related genes were indeed upregulated transiently on day 2 after the initiation of primary human myoblast differentiation. These include the upstream transcriptional master regulators of lipid metabolism, the nuclear hormone receptors PPARA, PPARG, RXRB, RXRG, NCOA1, NCOA2; the upstream fatty acid transporters FABP3, FABP4, CD36, SCARB1, FATP1-6; and a variety of lipases including LPL (FIG. 1F). Furthermore, we also observed a transient upregulation of FAO-related genes by day 2, including the rate-limiting carnitine palmitoyl-transferases CPT1A and CPT1B, the carnitine acetylase CRAT, an assortment of acyl-CoA dehydrogenases ACADs and hydroxyacyl-CoA dehydrogenases HADHs, and the mitochondrial electron transfer flavoproteins ETFA and ETFB, all of which are critical for mitochondrial FAO (FIG. 1G). It should be noted that these phenomena disappear by days 7-14 of primary human myoblast differentiation, strongly suggesting that mitochondrial FAO is transcriptionally upregulated only during the early phase of primary myoblast differentiation.

### Mitochondrial Metabolism During Early Differentiation of Human Myoblasts

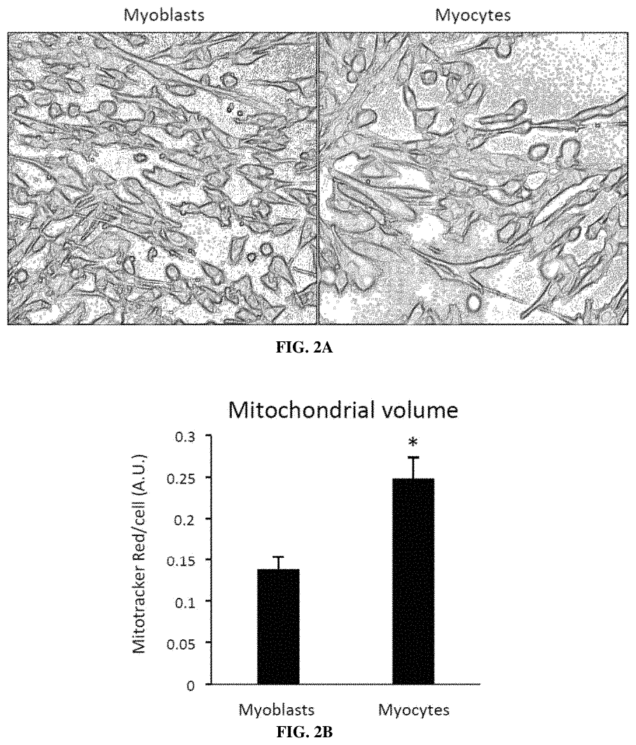

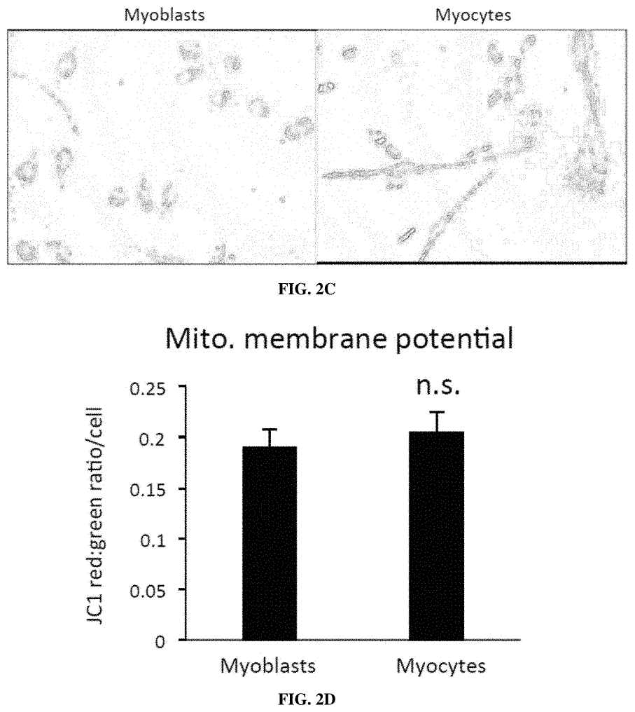

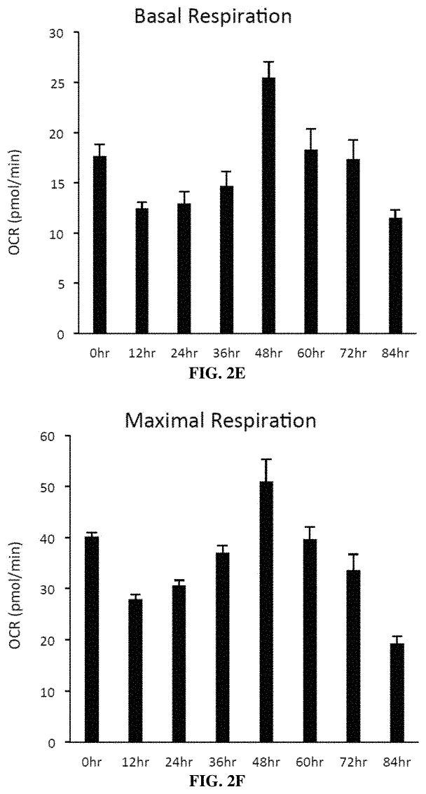

To test whether mitochondrial oxidation is indeed functionally increased in the early phases of myoblast differentiation, we stained the proliferative primary human myoblasts and non-proliferative myocytes with Mitotracker Red and JC1 dyes, to examine their mitochondrial volume and membrane potential. We found that just 48 hours after serum withdrawal, human myocytes manifested a significant increase in mitochondrial volume (FIGS. 2A and 2B). In contrast, we did not observe a significant change in mitochondrial membrane potential Δψₘ (FIGS. 2C and 2D), although both the JC1 red and JC1 green signals were increased (FIG. 7), consistent with the overall increase in mitochondrial volume. To verify if mitochondrial biogenesis was increased, we checked the mitochondrial DNA copy number, but found no significant changes at this early phase of myogenic differentiation (FIG. 8), suggesting the early increase in mitochondrial volume was not due to an increase in mitochondrial replication.

To examine if the early increase in mitochondrial volume translated to an increase in activity, we turned to the Seahorse Analyzer to measure basal and maximal O₂ consumption rates and assess the mitochondrial ETC flux and ATP synthesis rates in response to mitochondrial enzyme perturbations. Our analyses revealed that both the basal and maximal O₂ consumption rates initially dropped at 12 h of serum withdrawal, but rose quickly over time as differentiation progressed (FIGS. 2E and 2F). O₂ consumption rates peaked at ˜48 h of serum withdrawal, when non-proliferative myocytes had just formed. However, the O₂ consumption rates dropped significantly after 48 h of serum withdrawal, and continued in decline by 84 h (FIGS. 2E and 2F), when multinucleated myotubes were forming. Thus, in support of our earlier findings for FAO metabolism, our Seahorse Analyzer results indicated that mitochondrial oxidation transiently rose from 12-48 h during early myogenic differentiation, and declined from 48-84 h in the middle phase of myogenic fusion and differentiation, even though no significant changes in total nuclei number and total biomass were detected during the entire process.

### MyoD and PPARs Regulate the Transient Burst in FAO

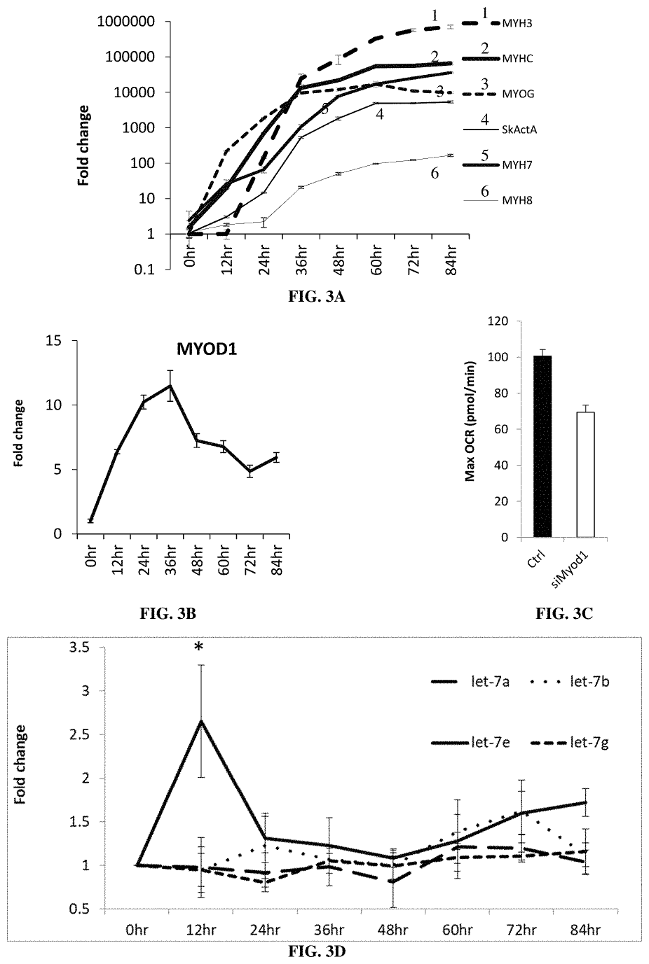

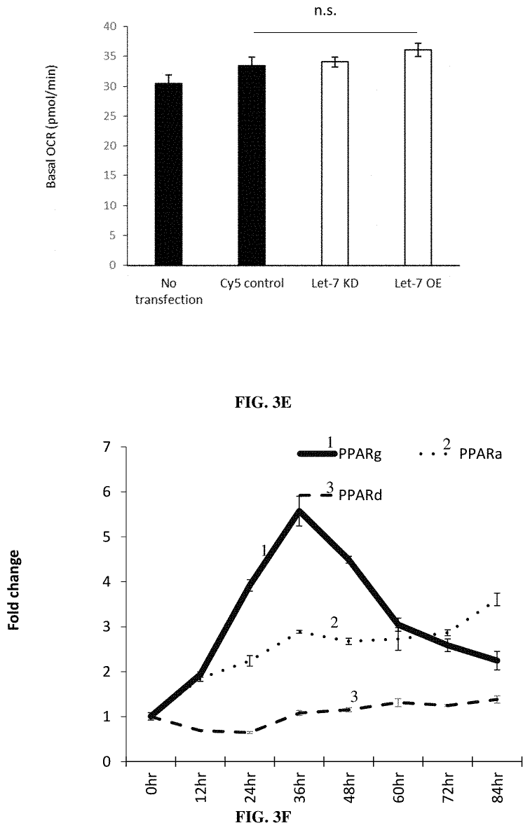

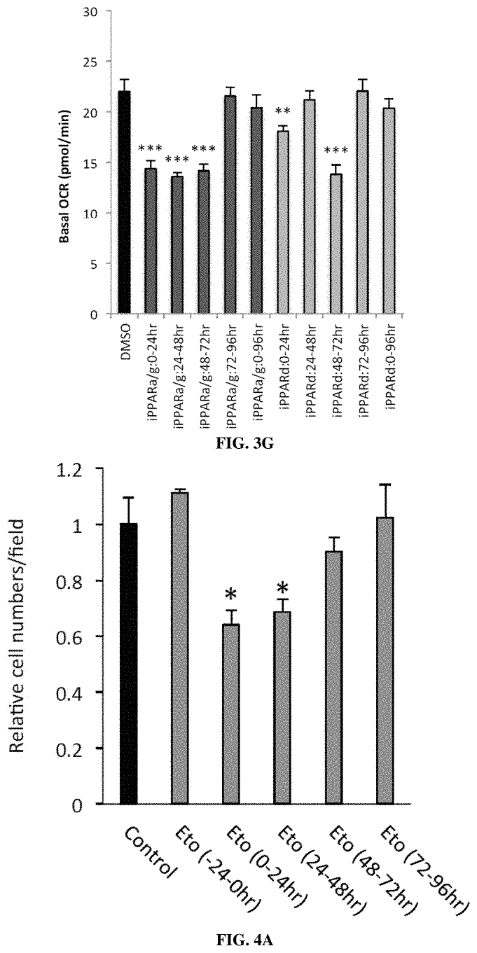

To dissect the mechanistic basis for the transient increase in mitochondrial FAO during the early stages of cellular differentiation, we performed RNA profiling for a variety of myogenic and differentiation regulators over a time-course in myogenic differentiation. Our results showed that most myogenic regulators either monotonically increased or decreased after serum withdrawal, thus eliminating them as candidates for driving the transient increase in mitochondrial FAO (FIG. 3A), with the exception of MYOD1. Only MYOD1 defied the monotonic trends and showed a transient increase between 12 h-36 h of myoblast differentiation (FIG. 3B). Interestingly, a study had also found that MyoD transactivates many mitochondrial oxidation genes to coordinate myogenesis (Shintaku et al., 2016). Consistent with this observation, when we knocked down human MYOD1 with siRNA, we could significantly reduce the maximal O₂ consumption rate in the human myocytes (FIG. 3C). However, MyoD siRNA failed to reduce the basal O₂ consumption rate (FIG. 9), which also significantly increased in human myocytes, suggesting that other regulators might be involved in promoting the early transient burst of mitochondrial FAO during myoblast differentiation.

Another class of metabolic regulators are the let-7 miRNAs (Zhu et al., 2011; Shyh-Chang et al., 2013; Jun-Hao et al., 2016), which are known to accumulate with differentiation across multiple cell-types in general. In particular, the let-7 miRNAs are also known to regulate insulin signalling in muscle cells and upregulate mitochondrial FAO by suppressing PI3K-mTOR signalling. During primary human myoblast differentiation, we found that let-7e miRNA did show a transient increase between 12-24 h of myoblast differentiation (FIG. 3D). However, when we knocked down let-7 miRNAs with LNA antagomiRs, the basal O₂ consumption rate remained unperturbed (FIG. 3E). When we overexpressed let-7 by artificial transfection, basal O₂ consumption rate increased slightly, but it was insignificant (FIG. 3E). Furthermore, maximal basal O₂ consumption rates decreased slightly after either let-7 overexpression or knockdown (FIG. 10). Thus, we concluded that let-7 miRNA upregulation was not an important reason behind the early transient burst of mitochondrial FAO in myocytes.

Finally, we turned to the master regulators of FAO, the Peroxisome Proliferator Activated Receptors (PPARs). Our profiling of the human myoblast differentiation time-course revealed that PPARγ mRNA underwent a transient increase from 0-36 h of myogenesis, declining back to near-basal levels by 84 h (FIG. 3F). This was confirmed at the protein level by Western blot (FIG. 12). PPARα also rose rapidly when PPARγ transiently increased at 0-36 h, but remained steady thereafter until 84 h. In contrast, PPARδ did not change significantly during human myoblast differentiation (FIG. 3F). By carefully applying PPAR inhibitors over a series of time-windows, we sought to test the PPAR mechanism in regulating the early transient burst of FAO. Our results revealed that PPARα/γ/δ were all required to maintain the increased basal and maximal O₂ consumption rates in human myocytes (FIGS. 3G and 11). In particular, we found that PPARα/γ inhibition from 0-72 h could significantly reduce basal and maximal respiration in myocytes, but not thereafter from 72 h-96 h (FIGS. 3G and 11). In contrast, PPARδ inhibition exerted a significant effect on basal respiration only at 0-24 h and 48-72 h, but not other time-windows (FIG. 3G). Interestingly, none of the PPAR inhibitors could reduce respiration when applied from 0-96 h throughout, suggesting that compensatory responses are activated to maintain the higher basal respiration in human myocytes when the PPARs are excessively inhibited. Taken together, our results suggest that the PPARs and MyoD play complementary roles in regulating mitochondrial FAO during early myoblast differentiation

### Controlling Myoblast Differentiation by Perturbing FAO at Different Times

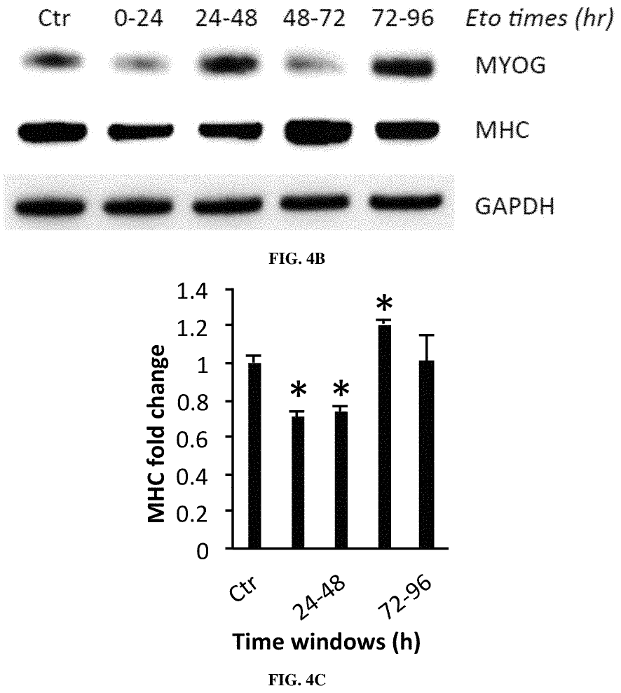

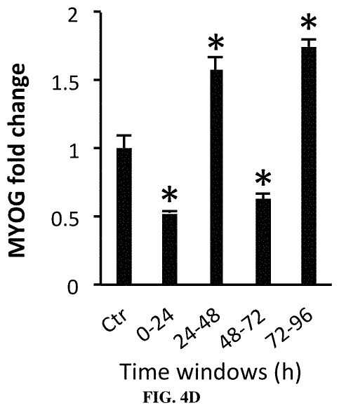

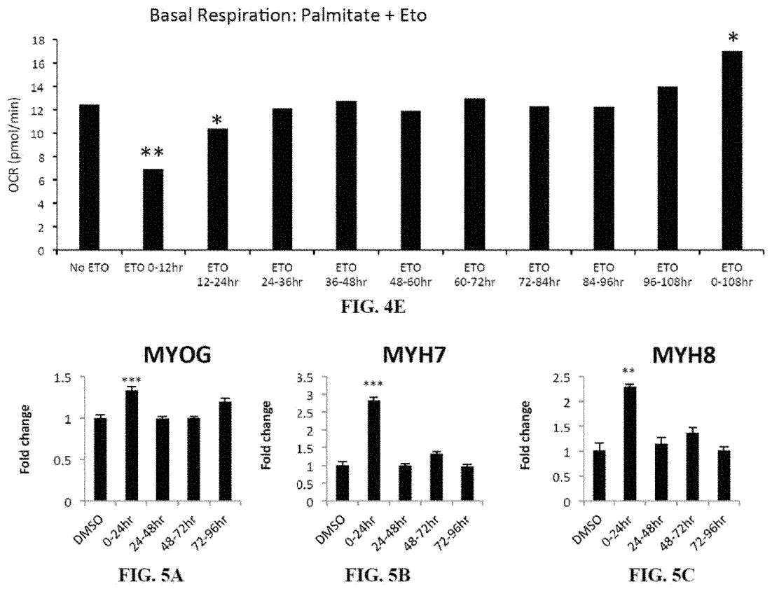

To test the importance of mitochondrial FAO in myoblast differentiation, we applied the mitochondrial CPT1-specific inhibitor etomoxir at low concentrations which avoid their off-target effects on CoA metabolism, during different time-windows of myoblast differentiation. We found that mitochondrial FAO inhibition severely compromised myocyte survival at 0-24 h and 24-48 h of myoblast differentiation (FIG. 4A), but not other time-windows, suggesting a transient but specific requirement for CPT1-mediated mitochondrial FAO between 0-48 h.

When the remaining adherent myocytes were assayed for changes in myocyte differentiation by Western blot, we found that mitochondrial FAO inhibition at different time-windows led to different profiles of myogenic markers (FIG. 4B). Quantification of the Western blots led us to conclude that 0-24 h mitochondrial FAO inhibition caused a MHC^{low}; MYOG^{low} phenotype (FIGS. 4B-4D), indicating that myogenic differentiation was blocked as a whole. 24 h-48 h mitochondrial FAO inhibition caused a MHC^{low}; MYOG^{high} phenotype (FIGS. 4B-4D), indicating that MYOG+ myocytes were now inhibited from fusing and differentiating into MHC+ myotubes. 48 h-72 h mitochondrial FAO inhibition caused a MHC^{high}; MYOG^{low} phenotype (FIGS. 4B-4D), indicating that MYOG+ myocytes could now fuse and differentiate into MHC+ myotubes, but some MYOG+ myocytes were also being depleted precociously. 72 h-96 h mitochondrial FAO inhibition caused a MHC^{high}; MYOG^{high} phenotype (FIGS. 4B-4D), indicating that late-stage mitochondrial FAO inhibition was actually enhancing myogenic differentiation instead. These results further demonstrate the idea that mitochondrial FAO is specifically required in the early stage of myoblast differentiation, and that FAO is inhibitory in the later stages of myogenesis.

When we performed Seahorse analysis on the O₂ consumption rates of human myocytes in response to mitochondrial FAO inhibition, we found that only the early 0-12 h and 12-24 h time-windows of etomoxir treatment could decrease O₂ consumption rates (FIG. 4E), further indicating that mitochondrial FAO is only specifically required during early myoblast differentiation. In later time-windows of etomoxir treatment, myocytes and myotubes appeared fully capable of utilizing other nutrient sources to fuel mitochondrial oxidation without detriment to survival or differentiation.

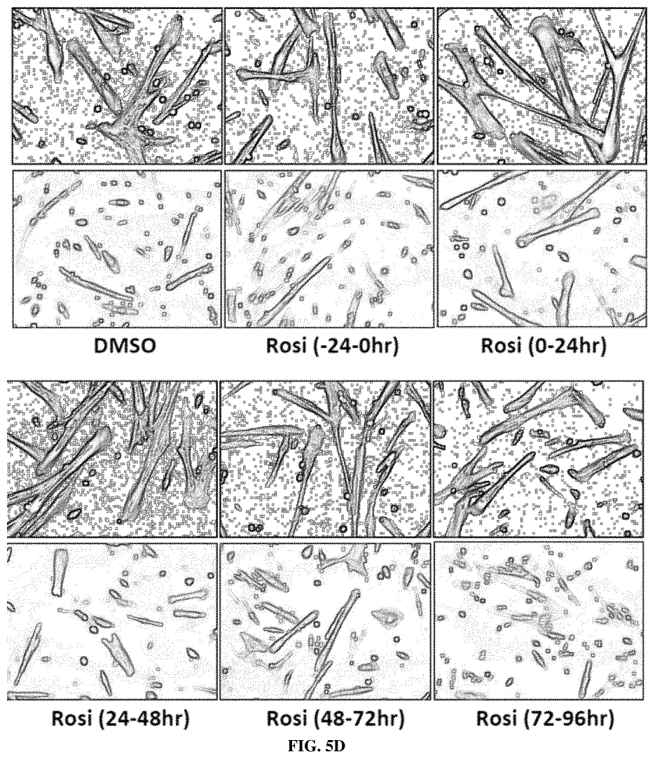

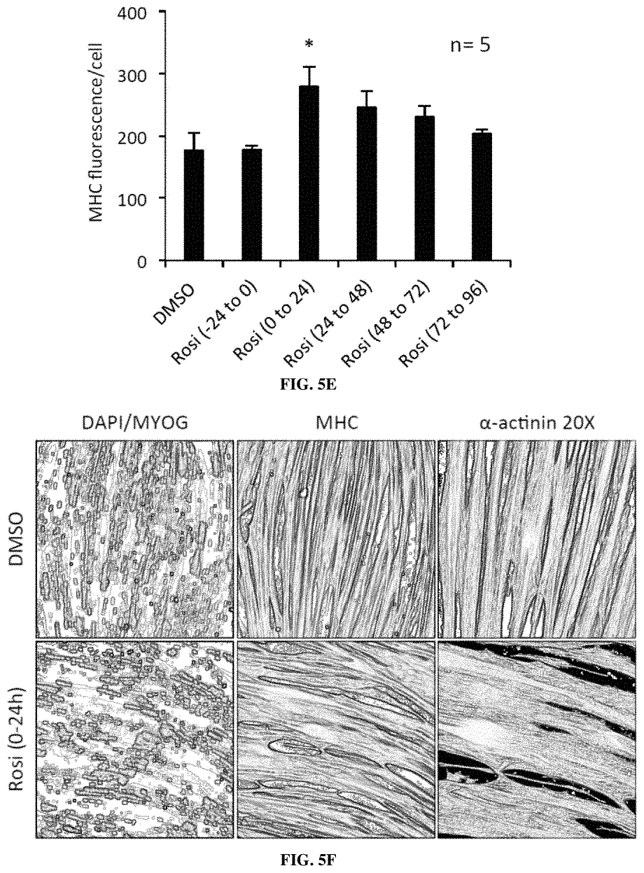

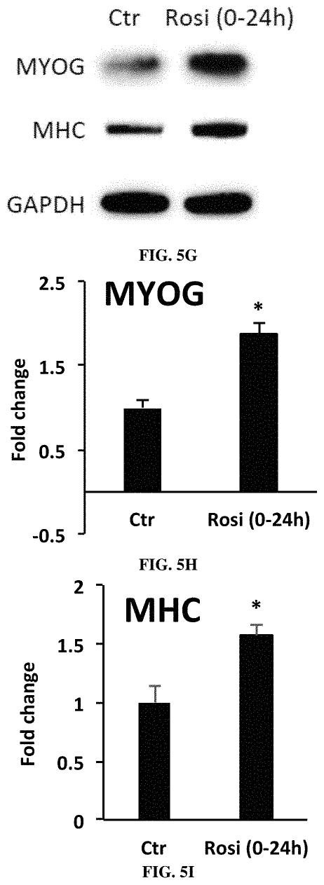

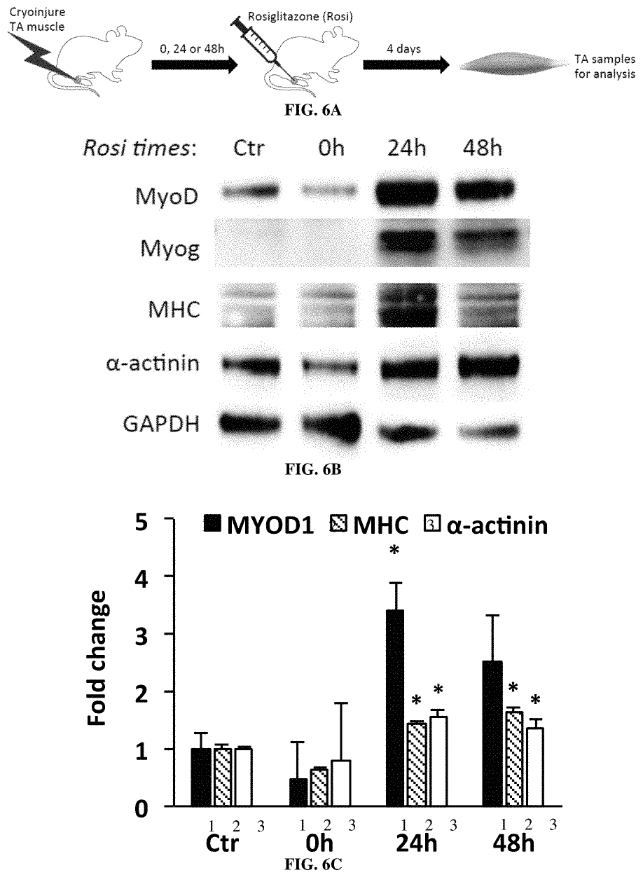

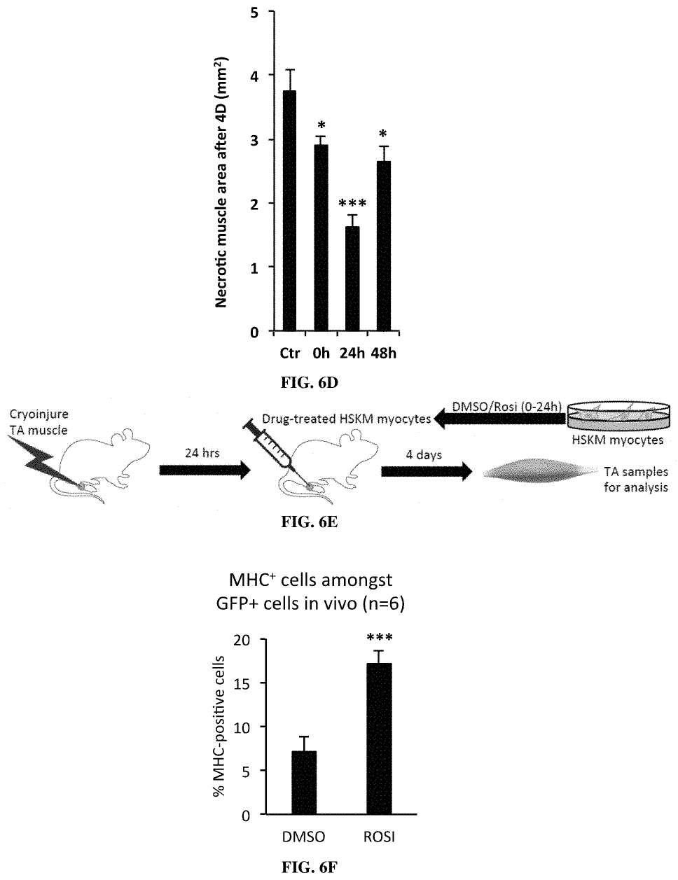

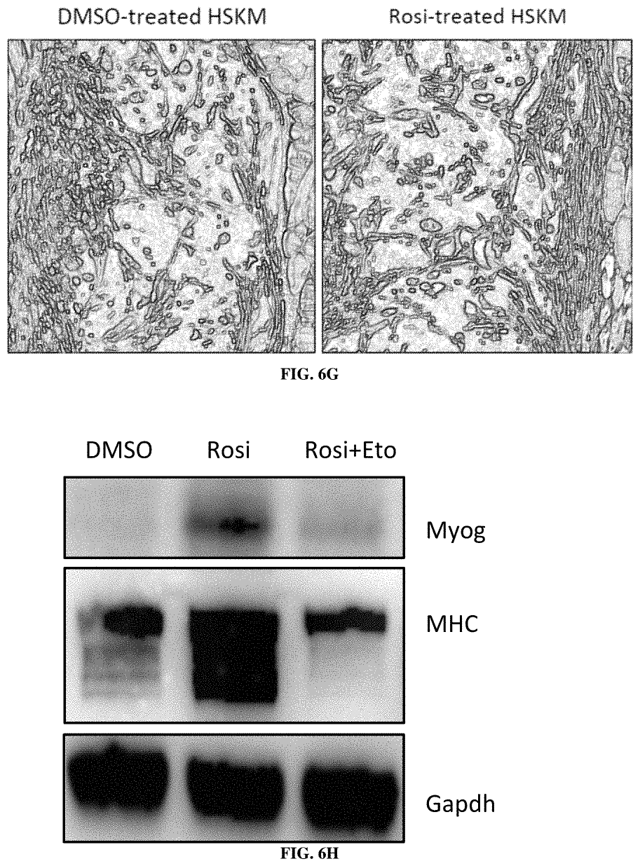

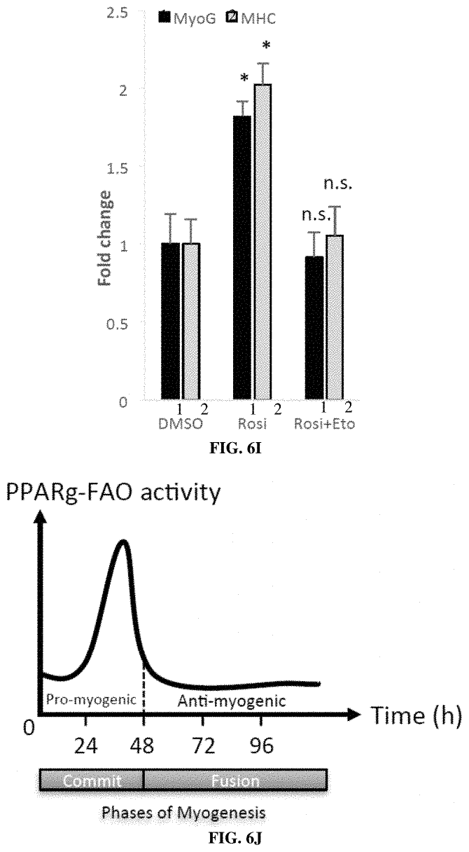

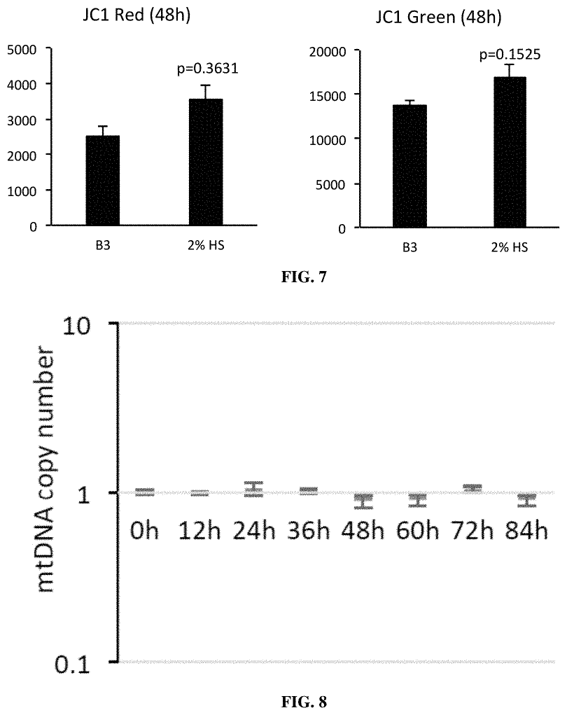

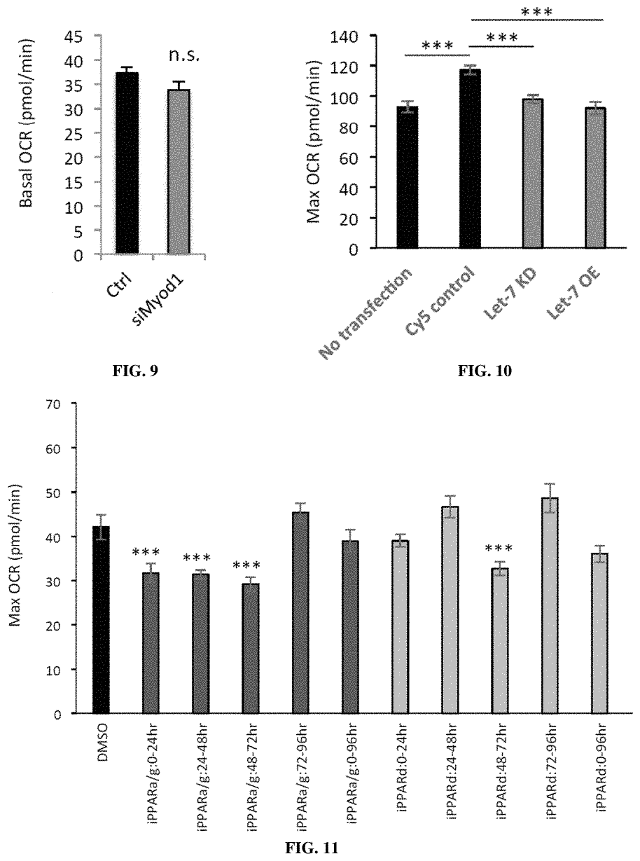

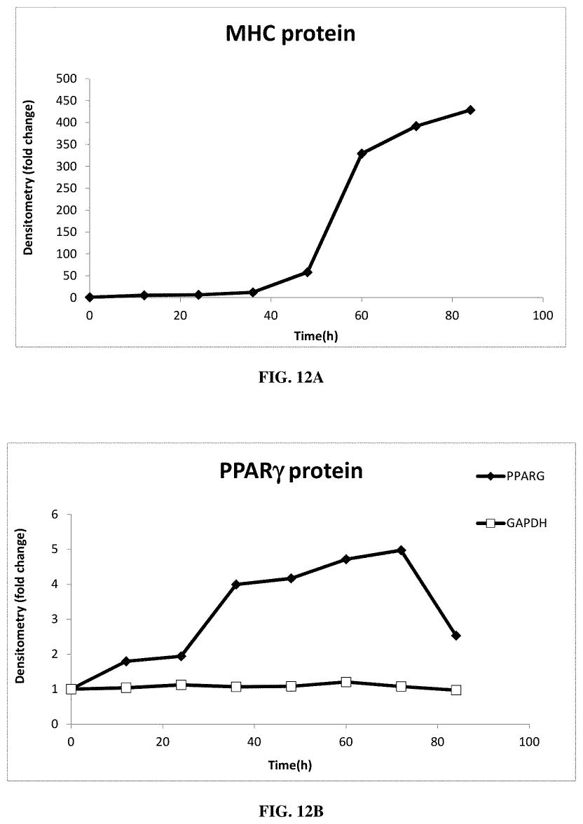

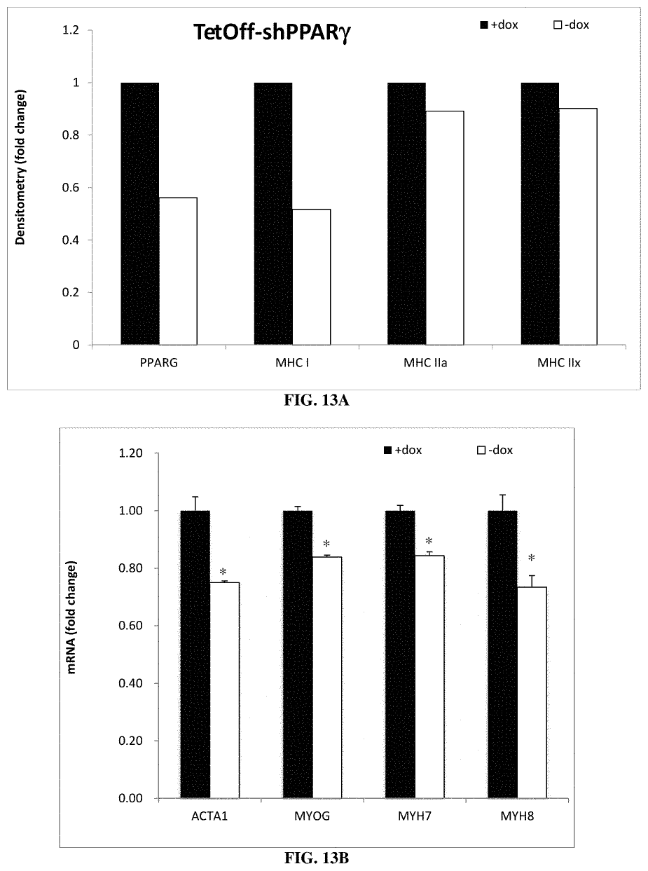

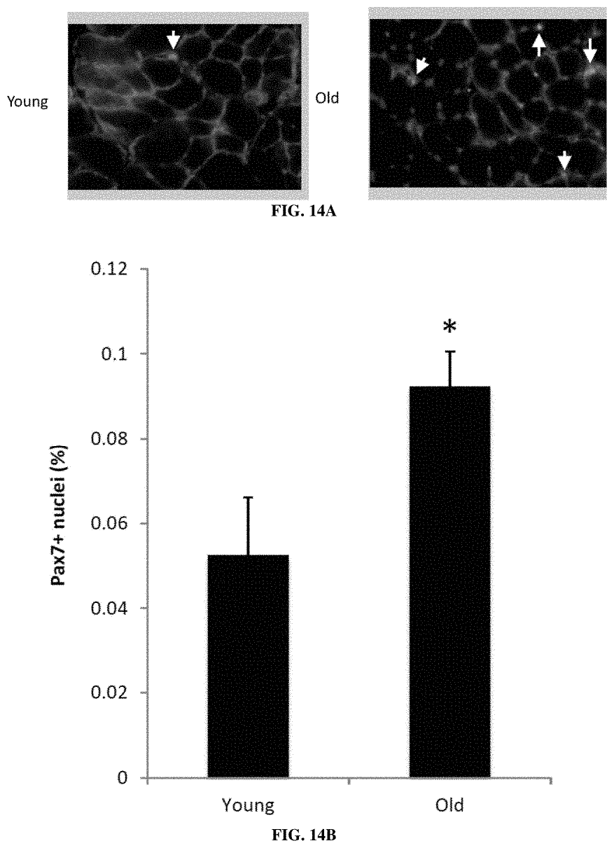

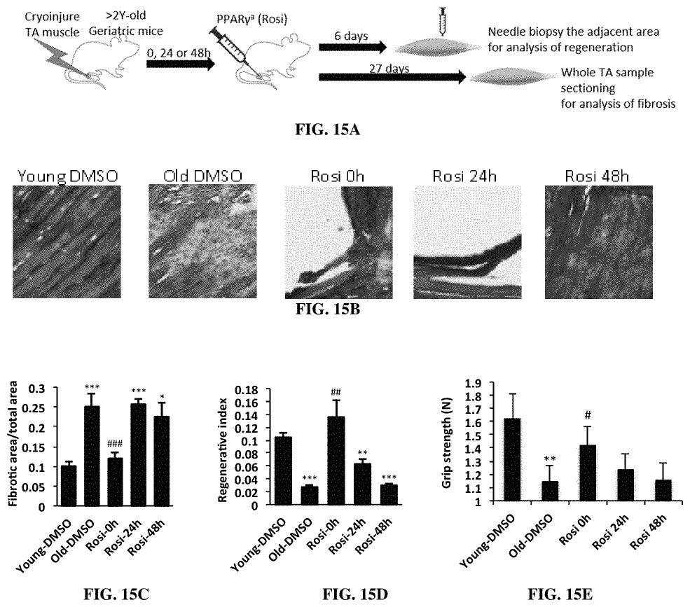

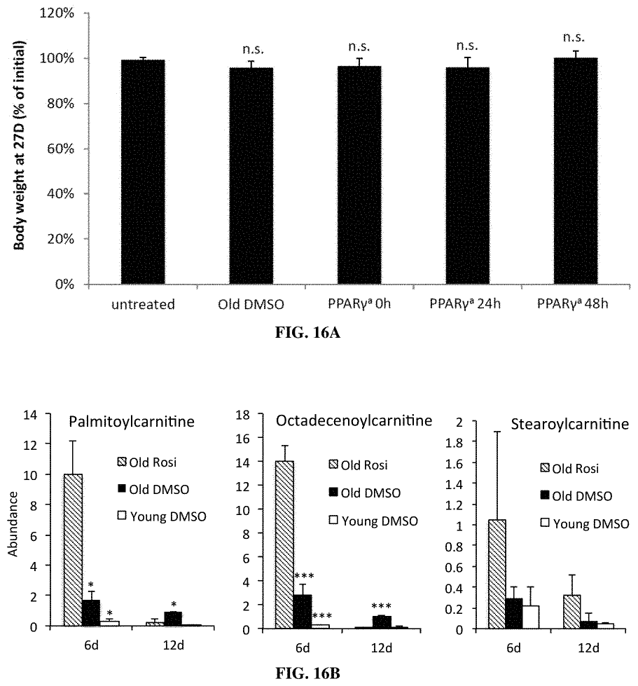

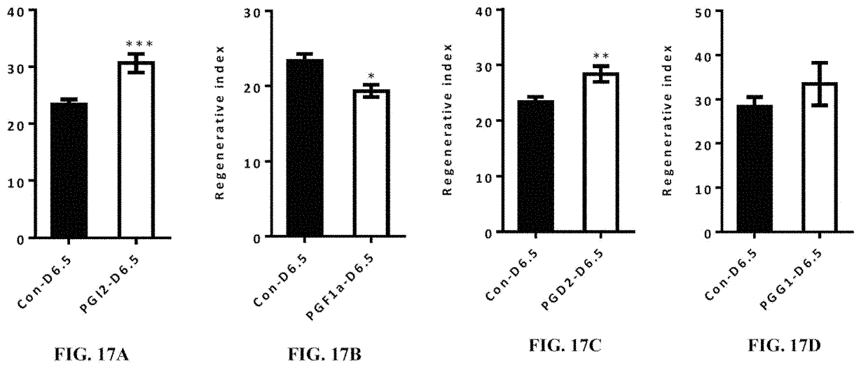

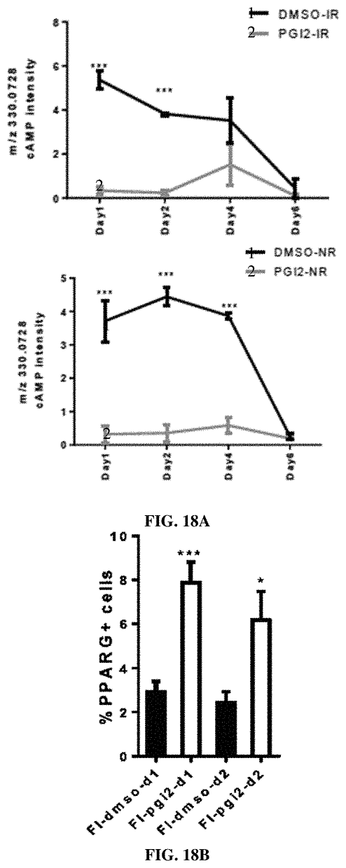

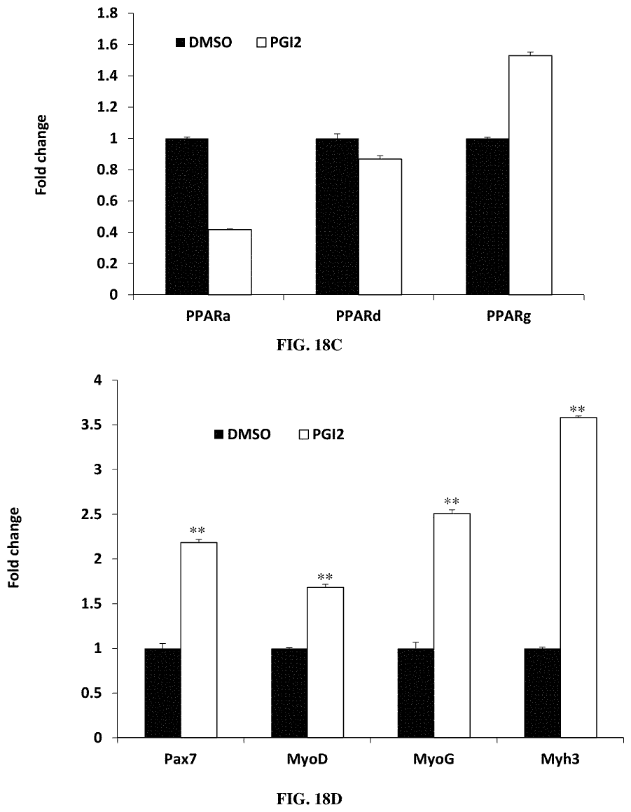

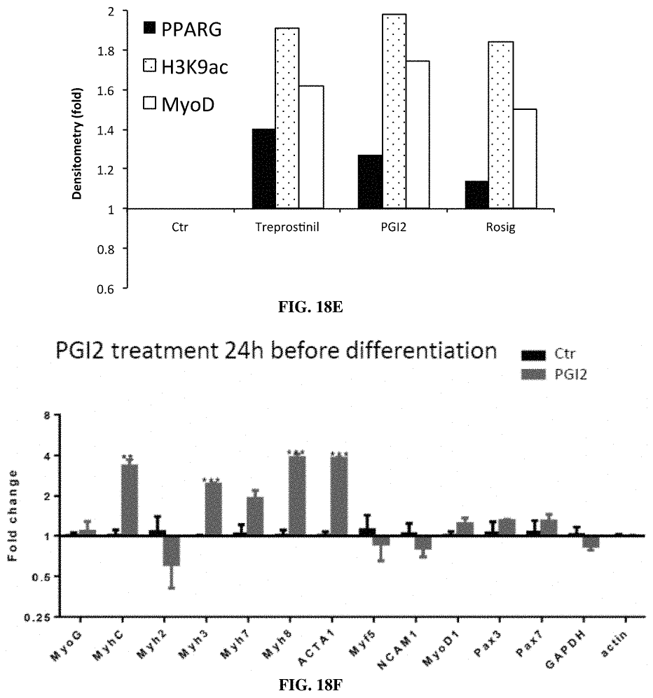

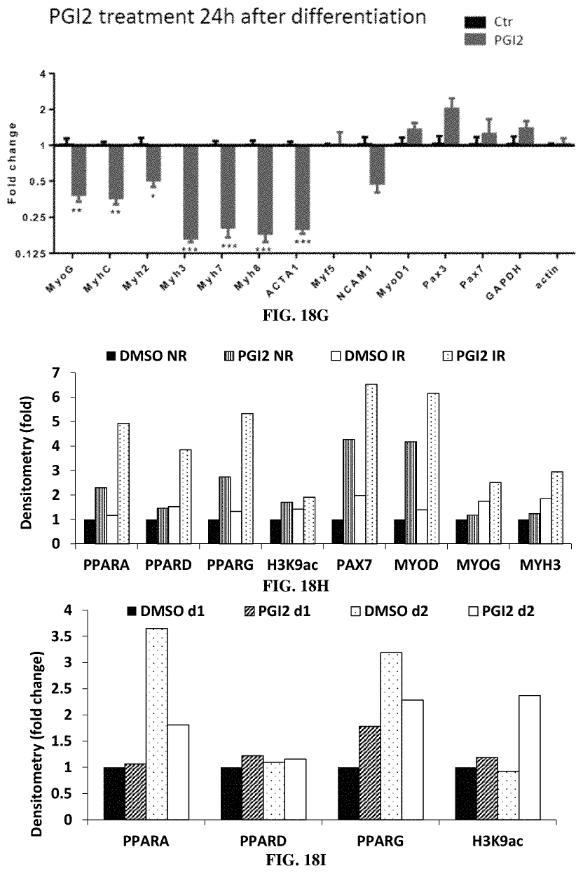

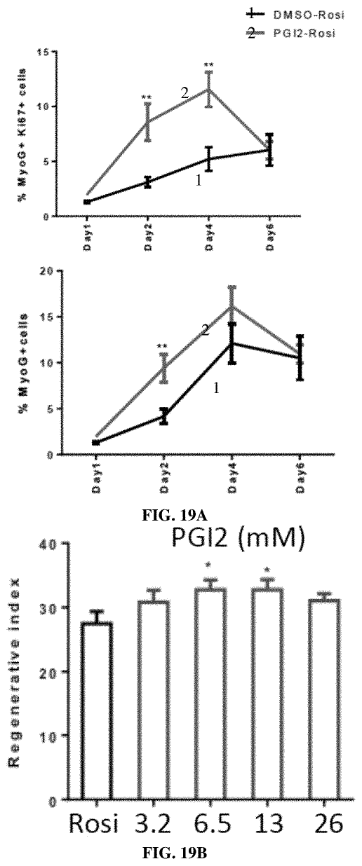

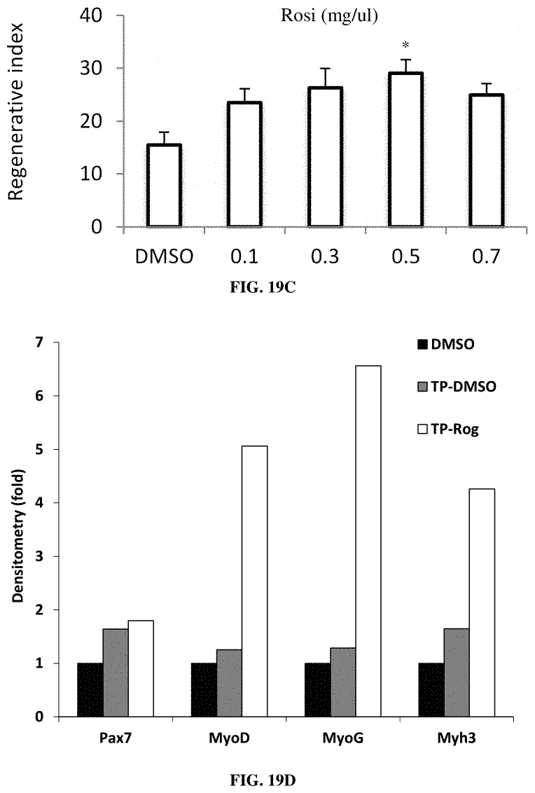

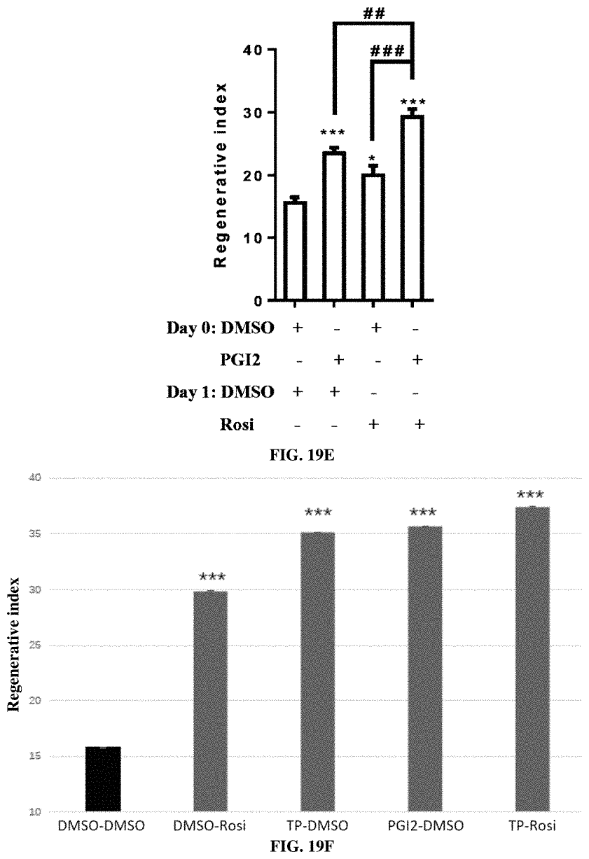

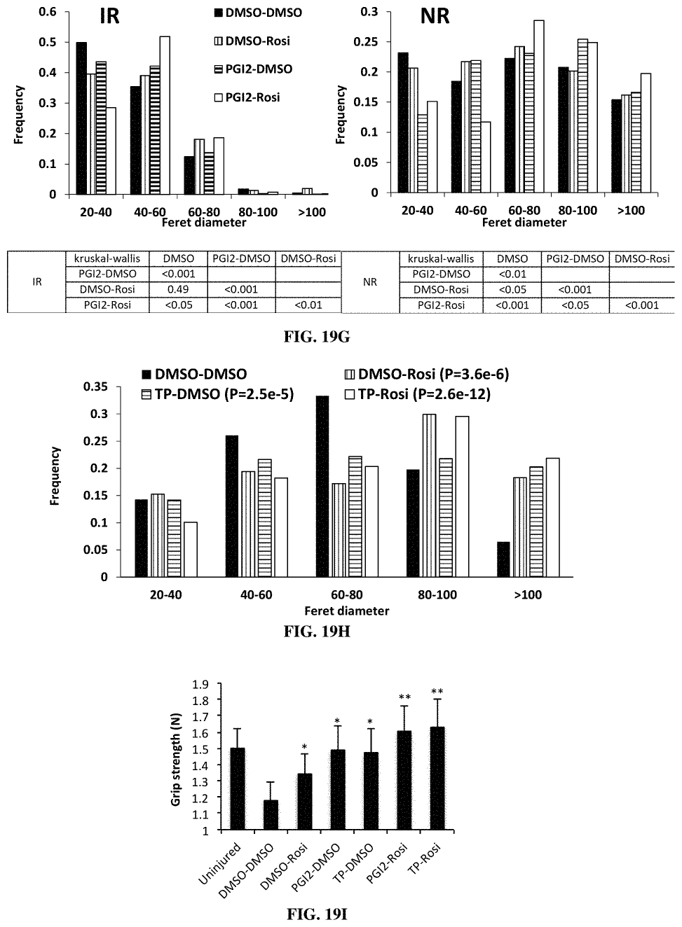

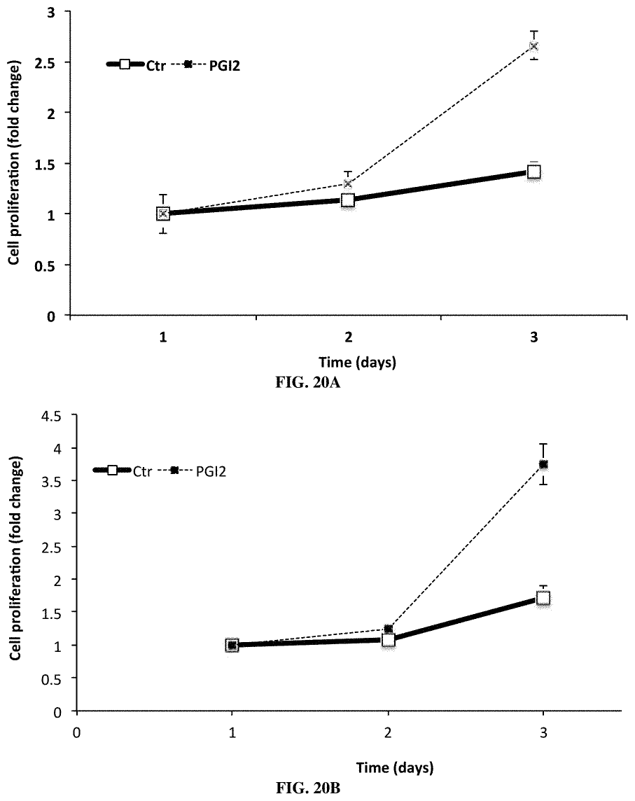

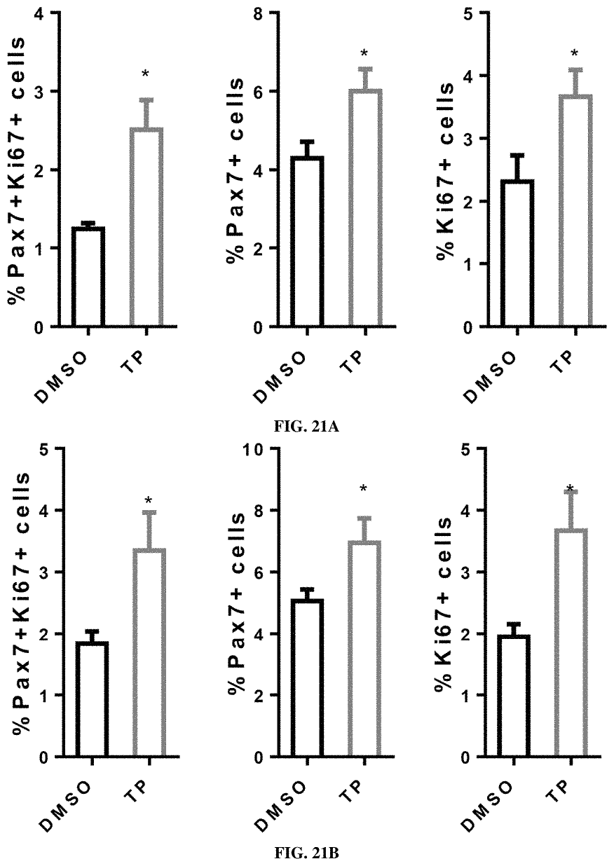

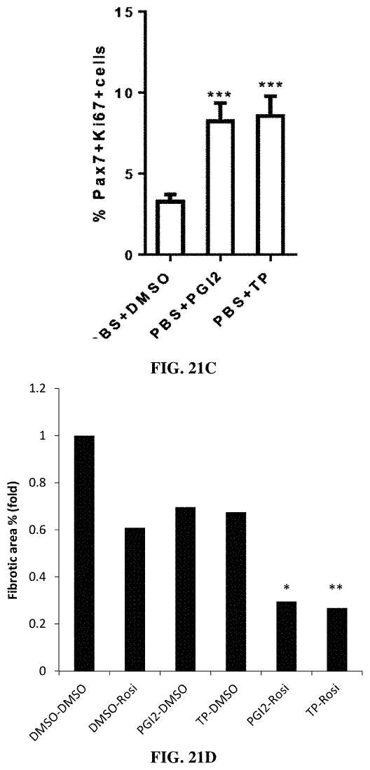

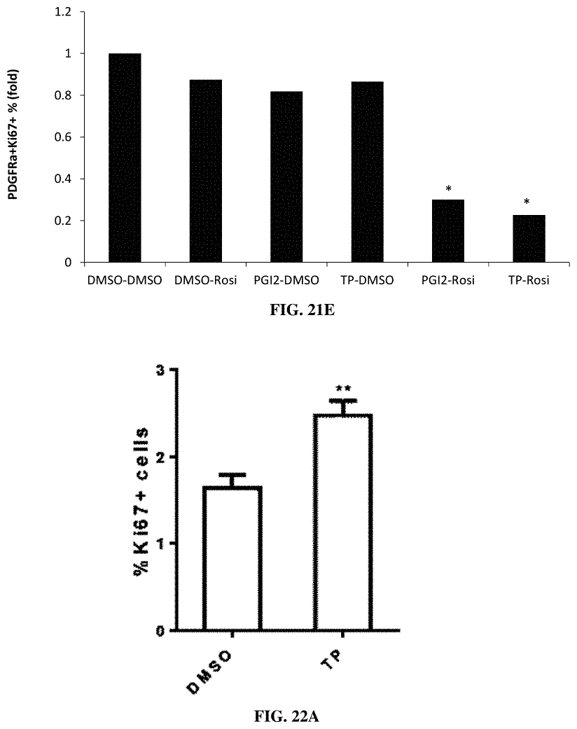

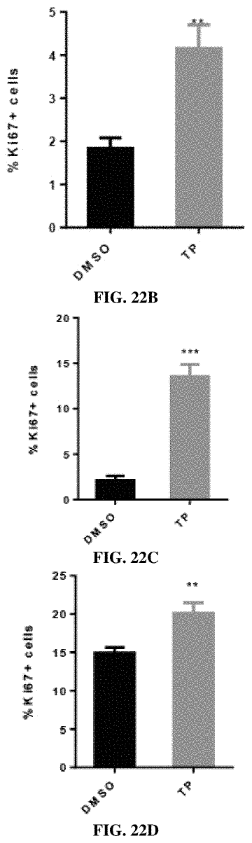

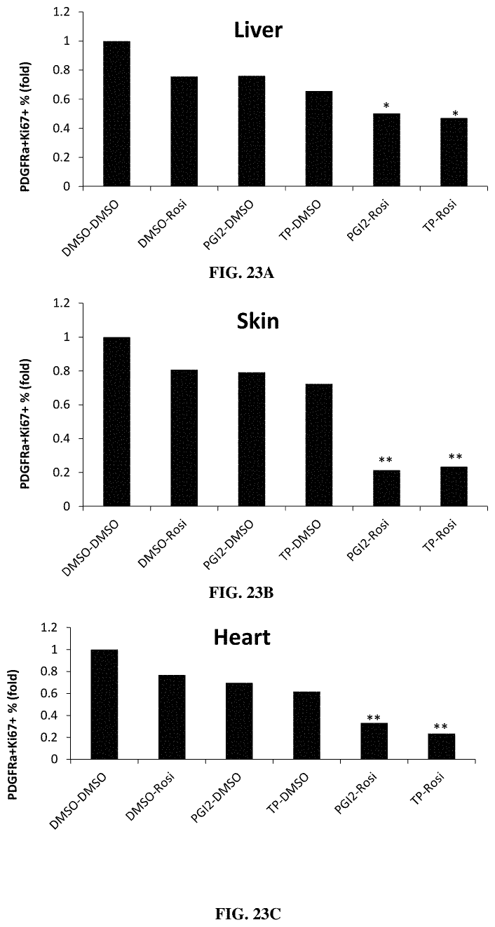

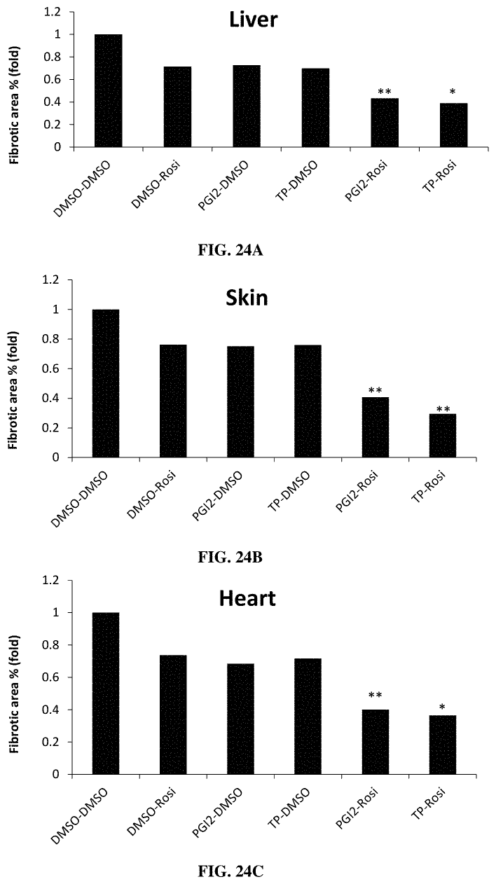

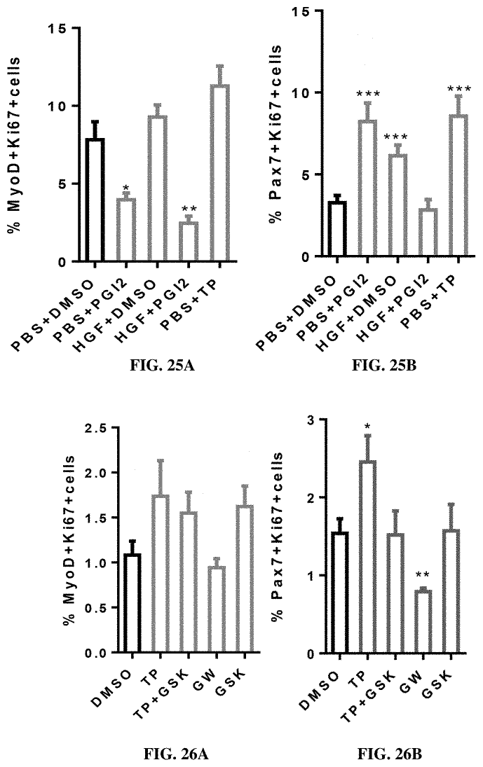

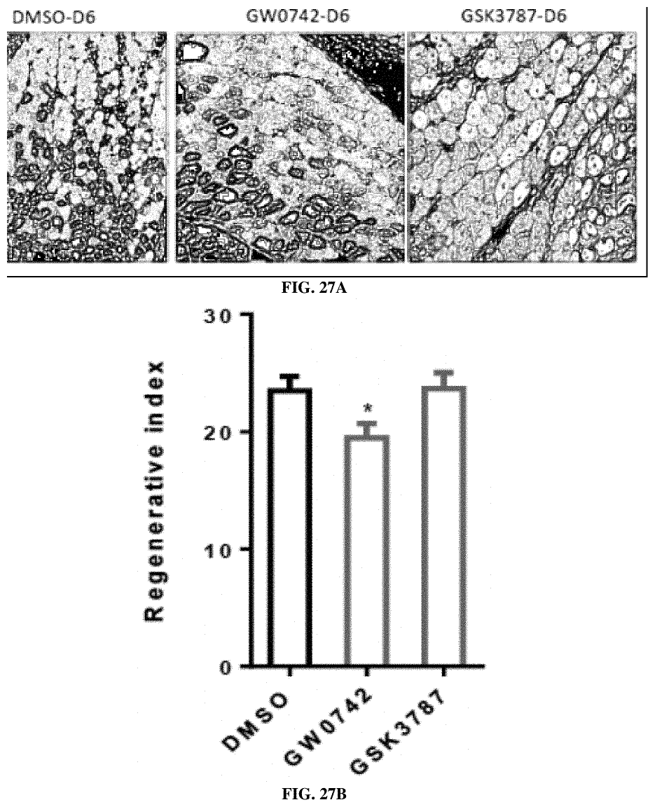

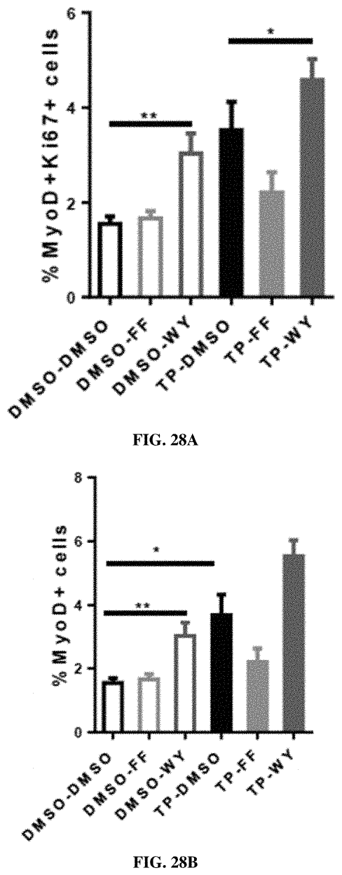

Having established the importance of the transient rise in PPARγ to mitochondrial FAO, and the importance of mitochondrial FAO to early myoblast differentiation, it was of interest to test if the anti-diabetic PPARγ agonists could enhance early myoblast differentiation. We tested the well-known thiazolidinedione Avandia, or rosiglitazone, during early human myoblast differentiation at low density. Our tests showed that rosiglitazone treatment at the 0-24 h time-window uniquely upregulated the mRNA levels of myogenin (MYOG), adult type I myosin heavy chain (MYH7) and perinatal myosin heavy chain (MYH8), whereas other time-windows of treatment had no significant effects at the end of 96 h (FIGS. 5A-5C). When the rosiglitazone-treated myocytes were immunostained for the myogenesis markers MHC protein and α-actinin, it was clear that rosiglitazone treatment at the 0-24 h and 24-48 h time-windows significantly enhanced myogenesis, but suppressed myogenesis in the other time-windows (FIGS. 5D and 5E). We repeated the optimal 0-24 h rosiglitazone treatment on human myocytes seeded at high density, and found that the resultant human myotubes were significantly more mature and hypertrophic than the control human myotubes (FIG. 5F). Quantification of MYOG and MHC protein expression by Western blot confirmed these observations (FIGS. 5G-5I). Thus, treatment with the PPARγ agonist at an early time-window could specifically enhance myogenic maturation. In contrast, Tet-repressible knockdown of PPARγ (PPARG) with a specific shRNA at the early phase (0-48 h) of primary human myoblast differentiation led to a reduction in several markers of myogenesis, including the myosin heavy chain proteins I, IIa, and IIx (FIG. 13A) and the mRNAs of ACTA1, MYOG, MYH7 and MYH8 (FIG. 13B). Thus PPARG is both necessary and sufficient for the early phase of myogenic differentiation.

### Enhancing Skeletal Muscle Regeneration In Vivo Via PPARγ-FAO

To test the utility of these in vitro findings in an in vivo context, we injected a bolus of the PPARγ agonist rosiglitazone directly into the cryoinjured skeletal muscles of mice at different time-points (FIG. 6A). We freeze-injured the tibialis anterior (TA) muscle of wildtype mice, then injected rosiglitazone into the injured TA muscle at 0 h, 24 h or 48 h after cryoinjury. 4 days after the initial cryoinjury, the TA muscle was harvested for analysis. Western blot analysis showed that intramuscular rosiglitazone injection at 24 h post-cryoinjury elicited the strongest expression of MyoD and MyoG protein (FIGS. 6B and 6C). Rosiglitazone injection at both 24 h and 48 h resulted in stronger expression of several MHC protein isoforms and α-actinin protein levels, relative to the DMSO control and 0 h time-window (FIGS. 6B and 6C). Quantification of the necrotic area confirmed that 24 h and 48 h injection of the PPARγ agonist rosiglitazone improved skeletal muscle regeneration in vivo (FIG. 6D).

To assess if these findings are clinically relevant to human myoblast transplant therapies (MTT; Chua et al., 2019), and to test if the effects of rosiglitazone on myoblasts are cell-autonomous, we pre-treated GFP+ human myoblasts under serum withdrawal conditions with either DMSO or rosiglitazone for the 0-24 h time-window, then orthotopically injected the human myoblasts into the TA muscle of immunodeficient NSG mice 24 h after cryoinjury (FIG. 6E). Our immunofluorescence analysis results showed that rosiglitazone pre-treatment significantly enhanced MHC protein expression among the engrafting human myocytes (FIGS. 6F and 6G), indicating that transient treatment with the PPARγ agonist rosiglitazone can enhance muscle regeneration mediated by human myoblast transplantation.

To confirm that these in vivo effects were dependent on the PPARγ-FAO axis, we tested rosiglitazone treatment against rosiglitazone+etomoxir treatment, relative to the DMSO control. Our results showed that the rosiglitazone-induced MYOG and MHC protein expression was abrogated by co-treatment with etomoxir (FIGS. 6H and 6I), indicating that the PPARγ-induction of myogenesis was dependent on mitochondrial FAO. Taken altogether, our experimental findings revealed that the PPARγ-FAO pathway had an unprecedented pro-myogenic role in the early phase of myogenesis during myocyte commitment (0-48 h; FIG. 6J).

To confirm that these findings were relevant to the regenerative defects often observed in aging skeletal muscles, we first checked if muscle stem cells are aberrant in the skeletal muscles of geriatric 2-year-old mice, which are equivalent to 60-year-old humans in biological terms. Immunostaining revealed that Pax7+ muscle stem cells were actually increased in aged mouse muscles (FIG. 14), suggesting that the regenerative defects in aged muscles were due to an aberrant decline in muscle stem cell differentiation, rather than an aberrant decline in muscle stem cell proliferative potential. Given our findings, we applied the PPARγ agonist rosiglitazone to geriatric 2-year-old mice. After cryoinjury of the TA muscles of >2 years-old geriatric mice, we intramuscularly injected a single bolus of the PPARγ agonist at different time-points, then assessed the regeneration and fibrosis of the TA muscles relative to young adult mice (FIG. 15A). As expected, Masson trichrome staining showed that the old mice had increased fibrosis after muscle regeneration, compared to young adult mice (FIG. 15B, 15C). Intramuscular PPARγ activation at the early 0 h time-point significantly reduced fibrosis, compared to the old DMSO control, the 24 h and the 48 h time-points (FIG. 15B, 15C). Consistent with these results, we found that old muscles showed a much lower regenerative index of embryonic MHC (eMHC)+ nuclei than young adult mice (FIG. 15D). We found that at the early 0 h time-point, a single PPARγ agonist injection significantly improved the old muscles' regenerative index, even surpassing young adult mice muscles (FIG. 15D). In contrast, intramuscular PPARγ activation at the 24 h and 48 h time-points did not show as strong an improvement in regenerative index. Although the optimal time-point shifted earlier in geriatric mice, compared to young adult mice, this could be due to higher levels of premature or pre-existing activation of Pax7+ muscle stem cells in old muscles (FIG. 14). The single bolus of PPARγ agonist was insufficient to affect total body weight and obesity (FIG. 16A), but did transiently increase FAO flux in the old TA muscle (FIG. 16B). Thus, these results confirm that PPARγ-induction of myogenesis can rejuvenate aged muscle regeneration, but only when administered at the early phase of myogenesis.

### Functional Assay of Prostaglandins in Tissue Regeneration

To test if other lipid mediators such as prostaglandins do exert significant functions in skeletal muscle regeneration, we injected a single bolus of the youth-associated prostaglandin PGI2 at 0 h post-injury into the TA muscle. Assessment of the regenerative index of eMHC+ nuclei revealed that PGI2 significantly promoted muscle regeneration, but only by day 6 (FIG. 17A). Injection of the prostaglandin PGF1a slightly decreased regeneration (FIG. 17B), the prostaglandin PGD2 slightly increased regeneration (FIG. 17C), whereas the prostaglandin PGG1 did not cause significant changes (FIG. 17D).

Novel Role of PGI2 in Regulating PPARG and Activating Stem Cells into Committed Progenitors

While GPCR-driven cAMP production is often thought to be the downstream mechanism of PGI2 signalling (Narumiya et al., 1999, DOI: 10.1152/physrev.1999.79.4.1193), the results show that cAMP was significantly decreased after PGI2 injection (***P<0.001, FIG. 18A), thus excluding the possibility that PGI2 could be exerting its pro-regenerative effects via cAMP signalling to protein kinase A (PKA), and supporting PGI2's mechanism via other targets. Previous work had shown that PGI2 can regulate PPARA and PPARD (Forman et al., 1997, DOI: 10.1073/pnas.94.9.4312; He et al., 2008, DOI: 10.1161/CIRCRESAHA.108.176057; Li et al., 2011, DOI: 10.1165/rcmb.2010-04280C), its role in regulating PPARG was unclear. Given our findings above, we checked to see if PGI2 administration could upregulate PPARG expression. Immunostaining revealed that a single bolus injection of PGI2 could indeed significantly increase PPARG+ cells (FIG. 18B) and also PPARG (but not PPARA nor PPARD) mRNA expression (FIG. 18C) in regenerating skeletal muscles. Concomitant with the increase in PPARG mRNA, several myogenic marker mRNAs such as Pax7, MyoD, MyoG and Myh3 were also significantly increased (FIG. 18D). To verify that PGI2 acts directly on myoblasts, we treated pure primary human myoblasts with a series of PGI2-related drugs. Western blots revealed that PGI2, the PGI2 analogue treprostinil and rosiglitazone could stabilize and/or upregulate PPARG protein levels (FIG. 18E). Simultaneously, a general marker for stem cell activation and progenitor commitment H3K9ac (acetylated histone H3 lysine 9), and the muscle progenitor-specific marker for stem cell activation MyoD, also followed the same pattern of regulation as PPARG, suggesting that PGI2-PPARG-driven FAO and acetyl-CoA synthesis promoted H3K9 acetylation and thus myoblast commitment. Consistent with this, PGI2 treatment of proliferative myoblasts (24 h pre-differentiation) increased several mRNA markers of myogenic differentiation including Myhc, Myh3, Myh8, and Acta1 (FIG. 18F), whereas PGI2 treatment of differentiating myoblasts (24 h post-differentiation) decreased several mRNA markers of myogenic differentiation including Myog, Myhc, Myh2, Myh3, Myh7, Myh8, Acta1 and increased some mRNA markers of myoblasts including MyoD1 and Pax3 (FIG. 18G). These results showed that long-term exposure to PGI2 promoted an intermediate state of committed myoblasts and blocked them from undergoing terminal differentiation. If true, an early bolus injection of PGI2 into injured muscles should transiently increase PGI2-PPARG-FAO-H3K9 signalling to activate muscle stem cells into committed myoblasts and myocytes, thus promoting muscle regeneration. Indeed, we found that a single bolus of PGI2 at 0 h post-injury significantly increased all myogenesis markers, including embryonic MHC (Myh3) protein expression by day 6 in both the injured region and non-injured region of regenerating TA muscles (FIG. 18H). Detailed analysis showed that the early bolus of PGI2 suppressed PPARA protein, increased PPARG protein, and H3K9ac levels as early as days 1-2 in the injured region of the regenerating muscles (FIG. 18I), further supporting our hypothesis.

### Novel Synergy Between PGI2 and PPARG in Promoting Tissue Regeneration

Time-course analysis of skeletal muscle regeneration confirmed that a single bolus injection of PGI2 at 0 h post-injury significantly increased the numbers of MyoG+Ki67+ committed myoblasts in the early phase of muscle regeneration (days 2-4; FIG. 19A). To optimize the concentration of PGI2, we performed a titration series of injections. Our results showed that the regenerative index did not increase linearly with PGI2 concentration, but saturated and peaked around 6.5-13 mM (FIG. 19B). Similarly, the regenerative index did not increase linearly with rosiglitazone concentration, but saturated and peaked around 0.5 mg/ul (FIG. 19C). Separately, we found that the PGI2 analogue treprostinil could also increase Pax7, MyoD, MyoG and Myh3 protein expression in regenerating muscles by day 6, indicating that PGI2 analogue drugs had a pro-regenerative effect similar to PGI2 in muscles (FIG. 19D). Because PGI2 signalling was likely already saturated at this concentrations of the PGI2 analogue, we added another injection of PPARG agonist to attempt to increase regeneration. Surprisingly, an additional injection of the PPARG agonist rosiglitazone at 24 h post-injury led to a dramatic increase in MyoD, MyoG and Myh3 protein expression (FIG. 19D), suggesting that PGI2 synergizes with PPARG agonists to promote muscle regeneration. Parallel testing of the optimal concentrations of PGI2 and rosiglitazone, either alone or in combination, confirmed that optimal PGI2 synergizes with optimal rosiglitazone to further enhance muscle regeneration (P<0.001, FIG. 19E). The PGI2 analogue treprostinil showed similar synergism results in regenerative index (FIG. 19F). Quantitation of the myofiber cross-sectional diameter in both the injured and uninjured regions confirmed that rosiglitazone in combination with PGI2 or the PGI2 analogue treprostinil can synergistically promote muscle hypertrophic growth (FIG. 19G, 19H) and grip strength (FIG. 19I) after injury.

PGI2-PPARG signalling Promotes Stem Cell Activation and Suppresses Tissue Fibrosis

Besides promoting muscle stem cells to activate and enter an intermediate state of committed myoblasts in early myogenesis, PGI2 signalling also promotes the proliferative capacity of myoblasts. Using pure primary human myoblasts, we found that long-term PGI2 treatment can significantly increase the proliferation rate of both early-passage (FIG. 20A) and late-passage myoblasts (FIG. 20B).

Furthermore, we found that 2 days after intra-peritoneal injection of a single bolus of the PGI2 analogue treprostinil, the fraction of Pax7+Ki67+ proliferative muscle stem cells, the total pool of Pax7+ muscle stem cells, and the total pool of Ki67+ proliferative cells in the gastrocnemius muscle were all significantly increased (P<0.05), even without injury (FIG. 21A). This was true for the quadriceps muscle (FIG. 21B) and the TA muscle as well, for both PGI2 and treprostinil (FIG. 21C). In addition, daily injection of PGI2, the PGI2 analogue treprostinil, or the PPARG agonist rosiglitazone, into aged and sarcopenic 2-year-old mice, led to a significant reversal of aging-induced fibrosis in the TA muscle. The decrease in fibrotic area was even greater when rosiglitazone was combined with PGI2 or the PGI2 analogue (FIG. 21D). Immunofluorescence of the muscle sections further revealed that the PGI2 analogue treprostinil, or the PPARG agonist rosiglitazone could all suppress PDGFRA+Ki67+ fibrotic precursor numbers. The decrease in aging-associated fibrotic precursors was even greater when rosiglitazone was combined with PGI2 or the PGI2 analogue treprostinil (FIG. 21E).

Treprostinil could also increase the total pool of Ki67+ progenitor cells in the endoderm-derived liver tissue, even without damage stimuli or injury (FIG. 22A), suggesting that PGI2 signalling could promote injury-free or wound-less tissue regeneration in multiple tissues of the body, beyond skeletal muscles. Indeed, besides the endoderm-derived liver tissue, treprostinil could also increase the total pool of Ki67+ progenitor cells in the mesoderm-derived heart and cardiac muscle tissue without injury (FIG. 22B), as well as the total pool of Ki67+ progenitor cells in the neuroectoderm-derived skin tissues (FIG. 22C) and hair follicles (FIG. 22D), even without damage stimuli or injury.

In addition daily injection of PGI2, the PGI2 analogue treprostinil, or the PPARG agonist rosiglitazone, into aged and sarcopenic 2-year-old mice, led to a significant reversal of aging-induced fibrosis in multiple non-skeletal muscle tissues, including the liver (FIG. 23A), skin (FIG. 23B), and heart (FIG. 23C). The decreases in fibrotic area were even greater when rosiglitazone was combined with PGI2 or the PGI2 analogue (FIG. 23A-C).

Immunofluorescence of multiple tissues further revealed that the PGI2 analogue treprostinil, or the PPARG agonist rosiglitazone could all suppress PDGFRA+ Ki67+ fibrotic precursor numbers in multiple non-skeletal muscle tissues, including the liver (FIG. 24A), skin (FIG. 24B), and heart (FIG. 24C).. The decrease in aging-associated fibrotic precursors was even greater when rosiglitazone was combined with PGI2 or the PGI2 analogue treprostinil (FIG. 24A-C).

Thus, drugs that modulate the PGI2-PPARG-FAO-H3K9ac axis could be useful for promoting general tissue regeneration and reversing fibrosis in multiple degenerative diseases, where tissue degeneration is occurring with or even without overt damage. Such degenerative diseases could include sarcopenia, cachexia, disuse atrophy, inflammatory myopathies, muscular dystrophies, cardiomyopathies, skin wrinkling, intractable cutaneous ulcers, skin wounds, bullosis, alopecia, keloids, dermatitis, macular degeneration, colitis, liver steatosis, steatohepatitis, liver fibrosis, cirrhosis, pancreatitis, type 2 diabetes (T2D), lipodystrophies, chronic obstructive pulmonary disease (COPD), pulmonary fibrosis, acute respiratory distress syndrome (ARDS), rheumatoid arthritis, osteoarthritis, osteoporosis, neurodegenerative diseases, cerebral infarction, myocardial infarction, pulmonary infarction, bone fracture, gastric ulcers, enteritis, chronic kidney disease, renal fibrosis, or any other genetically determined, environmentally determined or idiopathic disease processes causing loss or atrophy of tissue/organ/body part structure and function. Overall, our results suggest that the prostaglandin (PGI2)-PPAR-FAO-H3 acetylation pathway could be a general mechanism to mimick the effects of exercise and injury stimuli, to activate tissuegenic stem cells and drive regeneration in tissues derived from all three germ layers, including endoderm, mesoderm and neuroectoderm, and in both skeletal muscles and non-skeletal muscle tissues.

PGI2 Signalling Promotes Wound-Less Regeneration in Synergy with PPARG

Hepatocyte growth factor (HGF) has been previously shown to activate muscle stem cell proliferation (Tatsumi et al., 1998, DOI: 10.1006/dbio.1997.8803). To test if HGF synergizes with PGI2, we injected PGI2 with or without HGF into the gastrocnemius muscle. Our results showed that PGI2 combined with HGF failed to increase, while HGF alone and treprostinil (TP) alone could increase proliferative myoblasts (FIG. 25A) and proliferative muscle stem cells (FIG. 25B), thus excluding synergism between HGF and PGI2 signalling in activating stem cells.

PPARD has been shown to be a target of PGI2 in vascular cells (He et al., 2008, DOI: 10.1161/CIRCRESAHA.108.176057; Li et al., 2011, DOI: 10.1165/rcmb.2010-04280C). PPARD agonists have also been previously shown to be exercise mimetic drugs (Narkar et al., 2008; DOI: 10.1016/j.cell.2008.06.051). Our results showed that 2 days after injection into the gastrocnemius muscle, the PGI2 analogue (TP) alone significantly increased while the PPARD agonist GW0742 surprisingly decreased Pax7+ Ki67+ proliferative muscle stem cells (FIG. 26A). The PPARD inhibitor GSK3787 alone had no effect, but specifically ablated the stimulatory effect of TP when co-treated, suggesting that PPARD is partially necessary but insufficient to drive the stem cell activation effect of PGI2 and its analogs. Our results also surprisingly showed that the PGI2 analogue (TP) alone slightly increased MyoD+ Ki67+ proliferative myoblasts, with or without PPARD inhibition by GSK3787 (FIG. 26B). The PPARD agonist GW0742 alone had no effect, but the PPARD inhibitor GSK3787 alone slightly increased proliferative myoblasts, suggesting that PPARD is neither necessary nor sufficient to drive the stem cell activation effect of PGI2 and its analogs, but exerts complex feedback effects if inhibited.

To definitively test if PPARD plays a role in muscle regeneration, we injected a single bolus of the PPARD agonist GW0742 or the PPARD inhibitor GSK3787 into the injured TA muscle. The results showed that GW0742 can significantly decrease muscle regeneration (FIGS. 27A-27B), but not GSK3787, suggesting that PPARD does not drive skeletal muscle regeneration.

PPARA has also been shown to be a direct binding target of PGI2 (Forman et al., 1997, DOI: 10.1073/pnas.94.9.4312), and fenofibrate is a specific agonist of PPARA, while WY-14643 is an agonist of both PPARA and PPARG (EC50=0.63 and 32 uM respectively). Our results showed that the PGI2 analogue (TP) alone and WY-14643 alone significantly increased proliferative myoblasts (FIG. 28A, 30B). The PPARA agonist fenofibrate (FF) alone had no effect, but specifically ablated the stimulatory effect of TP when co-treated, suggesting that PPARA downregulation is necessary but insufficient to drive the stem cell activation effect of PGI2 and its analogs. In contrast, combined treatment of treprostinil (TP) with WY-14643 (WY) synergistically increased proliferative myoblasts even further (FIG. 28A, 28B), indicating that PGI2 signalling synergizes with PPARG not PPARA to activate stem cell proliferation.

### Discussion

Fatty acid oxidation is emerging as an important metabolic pathway that regulates cell fate. Downstream effects of FAO include bioenergetics-associated signalling via the AMP/ATP ratio, the NAD+/NADH ratio, redox stress signalling via mtROS, and, as shown here, the regulation of protein acetylation via acetyl-CoA (Shyh-Chang and Ng, 2017). Previous studies had shown that low levels of FAO are required for maintaining quiescent muscle stem cells (MuSCs), hematopoietic stem cells (HSCs), and intestinal stem cells (ISCs; Ryall et al. 2015; Pala and Tajbakhsh et al., JCS 2018; Ito et al., 2012; Mihaylova et al., 2018.) Conversely, we had previously shown that excessive mitochondrial FAO can induce excessive mtROS and p38 MAPK signalling to cause tissue atrophy during cachexia (Fukawa et al., 2016). After stem cells are activated to proliferate and then differentiate, it is known that the mitochondrial oxidative capacity would rise by the end of terminal differentiation (Remels et al., 2010; Wagatsuma and Sakuma 2013). However the intermediate changes, i.e. the oxidative kinetics, the upstream regulators, the precise nutrient source, and most importantly the cause vs. effect role of nutrient oxidation during each stage of tissuegenesis, had all remained unclear.

Here we found that FAO is surprisingly dynamic during tissuegenic differentiation. Under normal circumstances, mitochondrial oxidation drops during the first 24 h of myoblast differentiation, followed by a transient burst of FAO that is specifically required only for the early differentiation (24-48 h) into non-proliferative myocytes. Mechanistically, this early burst of FAO is driven by PPARγ. Subsequently, PPARγ-driven FAO is downregulated in the middle phases of differentiation into resting myotubes. Our findings complete the picture depicted by a recent study (Yucel et al., 2019), which only showed that mitochondrial oxidation transiently drops in the first 24 h of myogenic differentiation, possibly due to mitophagy (Sin et al., 2016). Our findings imply there are 2 waves of mitochondrial oxidation during cellular differentiation, and that the first wave driven by the PPARγ-FAO axis can be finely controlled to regulate stem cell fate and tissue regeneration, especially since the PPARγ-FAO axis represents an eminently druggable pathway at nearly every step. The second wave of increased mitochondrial oxidation is likely driven by PPARα (FIG. 3F; Biswas et al., 2016), which leads to significantly higher mitochondrial mass and oxidation at the end of terminal differentiation. Because aerobic exercise is a well-established method to transiently induce mitochondrial FAO in skeletal muscles and other tissues, our work has clear implications for how exercise metabolism might promote skeletal muscle and non-skeletal muscle regeneration after injuries.

The PPAR nuclear hormone receptors are well-known master regulators of lipid metabolism. Traditionally, PPARα is thought to be an activator of FAO in the liver, PPARβ/δ is a ubiquitous regulator of FAO in many tissues, whereas PPARγ is an activator of lipogenesis in various lipid-metabolizing tissues (Manickham and Wahli 2017). While generally true, several studies have begun to show that PPARγ could also upregulate FAO in other tissues (Benton et al., 2008; Sikder et al., 2018).

In fact, previous studies with PPARγ knockout and inhibition had led to conflicting conclusions on the role of PPARγ or lack thereof in skeletal muscle development and muscle insulin sensitivity (Hunter et al., 2001; Hevener et al., 2003; Norris et al., 2003; Singh et al., 2007; Dammone et al., Int J Mol Sci. 2018). Consistent with our findings with 0-96 h perturbation of PPARγ, muscle-specific PPARγ^{KO} mice revealed no overt phenotype in skeletal muscle development (Hevener et al., 2003; Norris et al., 2003), but myocyte triglyceride content was increased ˜50% (Hevener et al., 2003), suggesting that PPARγ promotes lipid catabolism in myocytes. Furthermore constitutive PPARγ^{KO} was found to increase the mitotic activity of primary mouse myoblasts in vitro, suggesting that PPARγ is necessary to block the proliferative state in myoblasts (Dammone et al., 2018). And while some studies showed that PPARγ activation is anti-myogenic (Hunter et al., 2001; Singh et al., 2007), others suggested that PPARγ inhibition is also anti-myogenic (Singh et al., 2007). Here our time-window experiments have clarified these conflicting findings by showing that excessive inhibition of any PPAR subtype can lead to compensatory responses, and that PPARγ-FAO is only pro-myogenic in early myoblast differentiation, becoming anti-myogenic in late myogenesis.

Yet another confounding factor is the difference between the immortalized C2C12 cell-line and primary muscle cells (Dressel et al., 2003; Hu et al., 2012). Our bioinformatics analyses showed that immortalized C2C12 already start with high levels of PPARγ initially, only downregulating PPARγ upon myogenic differentiation. In contrast to immortalized C2C12, primary myoblasts only transiently upregulate PPARγ during early differentiation, with important implications for our interpretations of immortalized C2C12 data. Regardless, previous studies had shown that the PPARs can regulate MyoD, and cooperate with MyoD to transactivate some myogenesis genes, including mitochondrial UCP3 (Hunter et al., 2001; Solanes et al., 2003). Furthermore it has been shown that MyoD can also cooperate with non-canonical NF-KB RelB to induce the transcription of PGC10 and a variety of oxidative genes, including FAO genes, in multi-nucleated myotubes (Shintaku et al., 2017). Consistent with these findings, the MyoD-RelB-PGC10 transcriptional network could be how MyoD promotes maximal OCR in myocytes, by upregulating the downstream mitochondrial oxidation machinery needed for maximal OCR. In contrast, PPARγ induction of the upstream fatty acid metabolism enzymes could feed mitochondrial FAO and upregulate the basal rates of OCR in primary human myocytes.

### REFERENCES

Anchelin, M., et al. (2011). PLoS One 6, e16955.
Baker, D. J., et al. (2011). Nature 479, 232-236.
Balzer, E., et al. (2010). Development 137, 891-900.
Bar-Nur, O., et al. (2018). Stem Cell Reports 10(5), 1501-1521.
Beausejour, C. M., et al. (2003). EMBO J. 22, 4212-4222.
Bernet, J. D., et al. (2014). Nat. Med. 20, 265-271.
Bigot, A., et al. (2008). Biol. Cell 100, 189-199.
Blau, H. M., et al. (1983). Proc. Natl. Acad. Sci. U.S.A 80, 4856-4860.
Blau, H. M., Cosgrove, B. D., and Ho, A. T. V (2015). Nat. Med. 21, 854-862.
Borchin, B., Chen, J., and Barberi, T. (2013) Stem Cell Rep. 1(6), 620-631.
Brack, A. S., and Rando, T. A. (2012). Cell Stem Cell 10, 504-514.
Briske-Anderson, et al. (1997). Proc. Soc. Exp. Biol. Med. 214, 248-257.
Buckingham, M., and Relaix, F. (2015). Cell Dev. Biol. 44, 115-125.
Capel, A. J., et al. (2019). Front. Bioeng. Biotechnol., 7, 20.
De Cecco, et al. (2019). Nature 566, 73-78.
Chandel, N. S., et al. (1998). Proc. Natl. Acad. Sci. U.S.A 95, 11715-11720.
Chandel, N. S., et al. (2000). J. Biol. Chem. 275, 25130-25138.
Chang, J., et al. (2016). Nat. Med. 22, 78-83.
Cheung, T. H., and Rando, T. A. (2013). Nat. Rev. Mol. Cell Biol. 14, 329-340.
Choi, Y., et al. (2019). Biomaterials 206: 160-169.
Christy, B., et al. (2015). p53 and rapamycin are additive. Oncotarget 6.
Chua, M.-W. J., et al. (2019). Assessment of different strategies for scalable production and proliferation of human myoblasts. Cell Prolif doi: 10.1111/cpr.12602.
Chung, C. Y., et al. (1999). Biochem. Biophys. Res. Commun. 265, 246-251.
Coletti, D., et al. (2002). EMBO J. 21, 631-642.
Conboy, I. M., and Rando, T. A. (2002). Dev. Cell 3, 397-409.
Conboy, M. J., et al., (2010). Protocols for Adult Stem Cells, Methods in Molecular Biology™ (Methods and Protocols), vol 621. Humana Press.
Copley, M. R., et al. (2013). Nat. Cell Biol. 15, 916-925.
Cosgrove, B. D., et al. (2014). Nat. Med. 20, 255-264.
Darabi, R., et al. (2008) Nat. Med. 14, 134-143.
Darwin, C., Darwin, F., and Darwin, F. (1887). The life and letters of Charles Darwin, including an autobiographical chapter. Edited by his son, Francis Darwin. (London, John Murray,).
Decary, S., Mouly, V., and Butler-Browne, G. S. (1996). Hum. Gene Ther. 7, 1347-1350.
Decary, S., et al. (1997). Hum. Gene Ther. 8, 1429-1438.
Demaria, M., et al. (2014). Dev. Cell 31, 722-733.
Deuchar, E. (1976). J. Embryol. Exp. Morphol. 35, 345-354.
Drummond, M. J., et al. (2011). Physiol. Genomics 43, 595-603.
Elsaeidi, F., et al. (2018). J. Neurosci. 38, 2246-2261.
Emerling, B. M., et al. (2005). Mol. Cell. Biol. 25, 4853-4862.
Emerling, B. M., et al. (2008). Proc. Natl. Acad. Sci. 105, 2622-2627.
Engleka, K. A., et al. (2005). Dev. Biol. 280, 396-406.
Esquenet, M., et al. (1997). J. Steroid Biochem. Mol. Biol. 62, 391-399.
Farr, J. N., et al. (2017). Nat. Med. 23, 1072-1079.
Flamini, V., et al. (2018). Stem Cell Reports 10, 970-983.
Fukawa, T., et al. (2016). Nat. Med. 22, 666-671.
Garcia-Prat, L., et al. (2016). Autophagy 12, 612-613.
Groskreutz, D. J., et al. (1994). J. Biol. Chem. 269(8), 6241-5.
Grounds, M. D. (2014). Bioarchitecture 4, 81-87.
Gussoni, E., Blau, H. M., and Kunkel, L. M. (1997). Nat. Med. 3, 970-977.
Gutscher, M., et al. (2008). Nat. Methods 5, 553-559.
Hanson, G. T., et al. (2004). J. Biol. Chem. 279, 13044-13053.
Hinds, S. et al. (2011). Biomaterials, 32(14), 3575-3583.
Huang, Y. (2012). Wiley Interdiscip. Rev. RNA 3, 483-494.
Hutcheson, D. A., et al. (2009). Genes Dev. 23, 997-1013.
Jeon, O. H., et al. (2017). Nat. Med. 23, 775-781.
Kastenhuber, E. R., and Lowe, S. W. (2017). Cell 170, 1062-1078.
Khodabukus, A. and Baar, K. (2009). Tissue Engineering Part C: Methods 15(3).
Kudlow, B. A., et al. (2008). Mol. Biol. Cell 19, 5238-5248.
L'honore, A., et al. (2014). Dev. Cell 29, 392-405.
L'honore, A., et al. (2018). Elife 7.
Lau, A., et al. (2019). J. Clin. Invest. 129, 4-11.
Le, M. T. N., et al. (2011). PLoS Genet. 7, e1002242.
Lee, S. J., et al. (2012). Proc. Natl. Acad. Sci. 109, E2353-E2360.
Li T. et al. (2018). Nano Research 11(10), 5240-5257.
Li, Z., et al. (2012). Dev. Cell 23, 1176-1188.
Liu, N., et al. (2017). Nat. Cell Biol. 19, 202-213.
Ma, X., et al. (2014). Nat. Commun. 5, 5212.
Majmundar, A. J., et al. (2010). Mol. Cell 40, 294-309.
Malhotra, J. D., and Kaufman, R. J. (2007). Antioxid. Redox Signal. 9, 2277-2294.
Mendell, J. R. (1995). Ann. Neurol. 37, 3-4.
Miwa, S., and Brand, M. D. (2003). Biochem. Soc. Trans. 31, 1300-1301.
Mullarky, E., and Cantley, L. C. (2015). Diverting Glycolysis to Combat Oxidative Stress.
Nakada, Y., et al. (2017). Nature 541, 222-227.
O'Driscoll, L., et al. (2006). J. Endocrinol. 191, 665-676.
Pajcini, K. V, et al. (2010). Stem Cell 7, 198-213.
Park, T. J., et al. (2017). Science. 356, 307-311.
Pearson, R. D. (1984). Acta Biotheor. 33, 51-59.
Poletto, M., et al. (2017). Nucleic Acids Res. 45, 10042-10055.
Porrello, E. R., et al. (2011). Science. 331, 1078-1080.
Price, F. D., v et al. (2014). Nat. Med. 20, 1174-1181.
Pronsato, L., et al. (2013a). Biocell 37, 1-9.
Pronsato, L., Boland, R., and Milanesi, L. (2013b). Arch. Biochem. Biophys. 530, 13-22.
Quintana, E., et al. (2008). Nature 456, 593-598.
Robinton, D. A., et al. (2019). Dev. Cell 48, 396-405.e3.
Rodier, F., Campisi, J., and Bhaumik, D. (2007). Nucleic Acids Res. 35, 7475-7484.
Ron, D., and Walter, P. (2007). Nat. Rev. Mol. Cell Biol. 8, 519-529.
Rowe, R. G., et al. (2016). J. Exp. Med. 213, 1497-1512.
Ryall, J. G., et al. (2015). Cell Stem Cell 16, 171-183.
Sadek, H. A., et al. (2014). Stem Cell Reports 3, 1.
Schienda, J., et al. (2006). Proc. Natl. Acad. Sci. 103, 945-950.
Schwarzkopf, M., et al. (2006). Genes Dev. 20, 3440-3452.
Semenza, G. L., et al. (1994). J. Biol. Chem. 269, 23757-23763.
Sena, L. A., and Chandel, N. S. (2012). Mol. Cell 48, 158-167.
Shah, M. V., Namigai, E. K. O., and Suzuki, Y. (2011). Mech. Dev. 128, 342-358.
Sharples, A. P., et al. (2011). J. Cell. Biochem. 112, 3773-3785.
Shelton, M., et al. (2016). Methods 101, 73-84.
Shinoda, G., et al. (2013). Stem Cells 31, 1563-1573.
Shyh-Chang, N., and Daley, G. Q. (2013). Cell Stem Cell 12, 395-406.
Shyh-Chang, N., et al. (2013a). Cell 155, 778-792.
Shyh-Chang, N., Daley, G. Q., and Cantley, L. C. (2013b). Development 140, 2535-2547.
Skuk, D., et al. (2010). Mol. Ther. 18, 1689-1697.
Smith-Bolton, R. K., et al. (2009). Dev. Cell 16, 797-809.
Sousa-Victor, P., et al. (2014). Nature 506, 316.
Stockley, T. L., et al. (2000). J. Lab. Clin. Med. 135(6): 484-92.
Takashima, Y., et al. (2016). Hepatology 64, 245-260.
Testa, S., et al. (2018). Materials today Communications 15: 120-123.
Tierney, M. T., et al. (2014). Nat. Med. 20, 1182-1186.
Tierney, M. T., et al. (2016). Cell Rep. 14, 1940-1952.
Urbach, A., et al. (2014). Genes Dev. 28, 971-982.
Van Raamsdonk, J. M., et al. (2002). J. Lab. Clin. Med. 139(1), 35-42.
Xipell, E., et al. (2016). Neuro. Oncol. 18, 1109-1119.
Xu, M., et al. (2018). Nat. Med. 24, 1246-1256.
Yang, M., et al. (2015). Development 142, 1616-1627.
Yao, K., et al. (2016). Cell Rep. 17, 165-178.
Yermalovich, A. V., et al. (2019). Nat. Commun. 10, 168.
Yin, H., Price, F., and Rudnicki, M. A. (2013). Physiol. Rev. 93, 23-67.
Yuan, J., et al. (2012). Science. 335, 1195-1200.
Yun, J., and Finkel, T. (2014). Cell Metab. 19, 757-766.
Zhang, J., et al. (2016). Cell Stem Cell 19, 66-80.
Zhu, H., et al. (2011). Cell 147, 81-94.

1. A method of promoting regeneration of a tissue, comprising contacting the tissue with one or more activators of fatty acid oxidation (FAO activators), wherein the tissue is selected from the group consisting of a muscle tissue, a liver tissue, a heart tissue, and a hair follicle.

2-5. (canceled)

6. The method of claim 1, wherein the tissue is contacted with the one or more FAO activators for no more than about 72 hours.

7. The method of claim 1, wherein the contacting is in vivo, in vitro, or ex vivo.

8. (canceled)

9. A method of treating a disease or condition associated with a tissue in an individual, comprising administering an effective amount of a pharmaceutical composition comprising a tissuegenic cell to the tissue of the individual, wherein the tissuegenic cell is contacted with one or more FAO activators prior to the administration of the pharmaceutical composition, and wherein the tissue is selected from the group consisting of a muscle tissue, a liver tissue, a heart tissue, and a hair follicle.

10-13. (canceled)

14. A method of treating a disease or condition associated with a tissue in an individual, comprising administering an effective amount of one or more FAO activators to the individual, wherein the tissue is selected from the group consisting of a muscle tissue, a liver tissue, a heart tissue, and a hair follicle.

15. The method of claim 9, wherein the disease or condition is tissue injury, tissue degeneration, tissue fibrosis, or aging.

16-19. (canceled)

20. The method of claim 14, wherein the tissue is an injured tissue.

21. The method of claim 20, wherein the one or more FAO activators is administered to the individual for no more than about 72 hours after the tissue injury.

22-23. (canceled)

24. The method of claim 1, wherein the tissue is a muscle tissue.

25. The method of claim 24, wherein the tissue comprises a tissuegenic cell, which is a myogenic cell.

26. The method of claim 25, wherein the myogenic cell is a myoblast and/or a myocyte.

27-36. (canceled)

37. The method of claim 1, wherein the one or more FAO activators comprise one or more activators of PPARγ.

38. A method of increasing FAO in a tissuegenic cell, comprising contacting the tissuegenic cell with one or more activators of PPARγ for no more than about 72 hours, wherein the tissuegenic cell is from a tissue selected from the group consisting of a muscle tissue, a liver tissue, a heart tissue, and a hair follicle.

39. A method of activating PPARγ in a tissuegenic cell, comprising contacting the tissuegenic cell with a prostaglandin selected from the group consisting of prostaglandin 12 (PGI2), prostaglandin D2 (PGD2), analogues, salts, solvates, tautomers, and stereoisomers thereof.

40-42. (canceled)

43. The method of claim 37, wherein the one or more activators of PPARγ comprise a thiazolidinedione, or derivative, salt, solvate, tautomer, or stereoisomer thereof.

44. The method of claim 43, wherein the thiazolidine is rosiglitazone, or a salt, solvate, tautomer, or stereoisomer thereof.

45. The method of claim 37, wherein the one or more activators of PPARγ comprise a prostaglandin selected from the group consisting of PGI2, PGD2, analogues, salt, solvate, tautomer, and stereoisomer thereof.

46. The method of claim 45,
i) wherein the prostaglandin is PGI2, or a salt, solvate, tautomer, or stereoisomer thereof; or
ii) wherein the prostaglandin is treprostinil, or a salt, solvate, tautomer, or stereoisomer thereof.

47. The method of claim 37, wherein the one or more activators of PPARγ are rosiglitazone and PGI2.

48. (canceled)

49. The method of claim 37, wherein the one or more activators of PPARγ are rosiglitazone and treprostinil.

50. (canceled)

51. The method of claim 37, wherein the one or more activators of PPART comprise i) a thiazolidinedione, or derivative, salt, solvate, tautomer, or stereoisomer thereof; and ii) a prostaglandin, or analogue, salt, solvate, tautomer, or stereoisomer thereof.


SEQUENCE LISTING (34 sequences)

SEQ ID NO: 1 - DNA, 20 bp, Homo sapiens
tggtatcgtggaaggactca

SEQ ID NO: 2 - DNA, 20 bp, Homo sapiens
cggcatgatggactacagcg

SEQ ID NO: 3 - DNA, 21 bp, Homo sapiens
ggggaaaactacctgcctgtc

SEQ ID NO: 4 - DNA, 20 bp, Homo sapiens
ttcattggggtcttggacat

SEQ ID NO: 5 - DNA, 21 bp, Homo sapiens
attgcttcgtggtggactcaa

SEQ ID NO: 6 - DNA, 20 bp, Homo sapiens
tgccacatcttgatctgctc

SEQ ID NO: 7 - DNA, 20 bp, Homo sapiens
taaacacacctgcctgatgc

SEQ ID NO: 8 - DNA, 25 bp, Homo sapiens
cgacatcaggaaggacctgtatgcc

SEQ ID NO: 9 - DNA, 22 bp, Homo sapiens
tcggcgaggatagttctggaag

SEQ ID NO: 10 - DNA, 22 bp, Homo sapiens
ggcttccactacggtgttcatg

SEQ ID NO: 11 - DNA, 22 bp, Homo sapiens
agcctgcgaaagccttttggtg

SEQ ID NO: 12 - DNA, 20 bp, Homo sapiens
ttcagctcagggatgacctt

SEQ ID NO: 13 - DNA, 19 bp, Homo sapiens
caggcagtctaggctcgac

SEQ ID NO: 14 - DNA, 20 bp, Homo sapiens
aggcgctcgatgtactggat

SEQ ID NO: 15 - DNA, 20 bp, Homo sapiens
aacgtccactcaatgccttc

SEQ ID NO: 16 - DNA, 21 bp, Homo sapiens
ggccatgtcttcgatcctgtc

SEQ ID NO: 17 - DNA, 20 bp, Homo sapiens
ctcggcttcaaggaaaattg

SEQ ID NO: 18 - DNA, 24 bp, Homo sapiens
tcagctttaacaggaaaataaacg

SEQ ID NO: 19 - DNA, 23 bp, Homo sapiens
ggcctcgtcgtactcctgcttgg

SEQ ID NO: 20 - DNA, 22 bp, Homo sapiens
gaccacaggataagtcaccgag

SEQ ID NO: 21 - DNA, 22 bp, Homo sapiens
ctggcacttgttgcggttcttc

SEQ ID NO: 22 - DNA, 23 bp, Homo sapiens
ggcttcacattcagcaaacctgg

SEQ ID NO: 23 - DNA, 22 bp, Homo sapiens
cactgaaaaagatgagtatgcc

SEQ ID NO: 24 - DNA, 24 bp, Homo sapiens
acgccataaaactcttcaccaaag

SEQ ID NO: 25 - DNA, 22 bp, Homo sapiens
accttggctatcatcacccgat

SEQ ID NO: 26 - DNA, 22 bp, Homo sapiens
agttacaatcggcatcaaccaa

SEQ ID NO: 27 - DNA, 22 bp, Homo sapiens
tggggttagcgatggaggtagg

SEQ ID NO: 28 - DNA, 20 bp, Homo sapiens
cactttccacacagacatca

SEQ ID NO: 29 - DNA, 19 bp, Homo sapiens
aacattccctgacaatccc

SEQ ID NO: 30 - DNA, 24 bp, Homo sapiens
gggttcatagtagaagagcgatgg

SEQ ID NO: 31 - DNA, 21 bp, Homo sapiens
agtgcgatgagtaggggaagg

SEQ ID NO: 32 - DNA, 23 bp, Homo sapiens
cccggagcacataaatagtatgg

SEQ ID NO: 33 - DNA, 22 bp, Homo sapiens
aataggatcctcccgaatcaac

SEQ ID NO: 34 - DNA, 20 bp, Homo sapiens
tggttaggctggtgttaggg